(19) United States
(12) Patent Application Publication
(10) Pub. No.: US 2002/0061862 A1
(43) Pub. Date: May 23, 2002

(54) Inducible nitric oxide synthase for treatment of disease

(75) Inventors: Timothy R. Billiar, Nevillewood, PA (US); Edith Tzeng, Pittsburgh, PA (US); David Geller, Pittsburgh, PA (US); Richard L. Simmons, Pittsburgh, PA (US); Larry L. Shears II, Bethel Park, PA (US); Andreas K. Nussler, Ulm (DE)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: University of Pittsburgh of the Commonwealth System of Higher Education, Pittsburgh, PA

(21) Appl. No.: 09/992,056

(22) Filed: Nov. 13, 2001

Related U.S. Application Data
(63) Continuation of application No. 09/176,496, filed on Oct. 21, 1998.
Application No. 09/176,496 is a continuation-in-part of application No. 08/465,522, filed on Jun. 5, 1995.
Application No. 08/465,522 is a division of application No. 08/314,917, filed on Sep. 28, 1994.
Application No. 08/314,917 is a continuation of application No. 07/981,344, filed on Nov. 25, 1992, now abandoned.
Application No. 08/314,917 is a continuation of application No. 08/745,375, filed on Nov. 8, 1996.
Application No. 08/745,375 is a continuation-in-part of application No. 08/630,798, filed on Apr. 10, 1996, now abandoned.
Application No. 08/630,798 is a continuation-in-part of application No. 08/265,046, filed on Jun. 24, 1994.

(51) Int. Cl. A61K 48/00; A61K 9/127; C12N 15/861

(52) U.S. Cl. 514/44; 435/320.1; 424/450; 435/456

(57) ABSTRACT

The invention provides a pharmaceutical composition comprising as an active ingredient a pharmaceutical agent comprising a DNA sequence that codes for a protein which possesses the biological activity of inducible nitrogen monoxide synthase (iNOS) and eukaryotic regulation elements, wherein the eukaryotic regulation elements result in the expression of said DNA sequence in eukaryotic cells, and a pharmaceutically acceptable carrier. The pharmaceutical agent can be complexed to liposomes.

This application in a continuation-in-part of U.S. application Ser. No. 08/265,046, filed Jun. 24, 1994, now pending.

The invention described herein was made in the course of work supported in part by Public Health Service, Grant Nos. GM44100 and GM37753 from the United States National Institutes of Health, General Medical Sciences. The United States Government has certain rights in this invention.

### 1. INTRODUCTION

The present invention relates to the use of a nucleic acid sequence encoding inducible NOS (iNOS) or a biologically active iNOS protein fragment in gene therapy treatment of mammalian host diseases or disorders. Such maladies include. but are not limited to, treatment of vascular occlusive disease. as well as cancer, microbial infection, inflammation, induced tissue injury and non-healing wounds.

The present invention also relates to optimization of the local effect imparted by means of iNOS expression within target cells by tandem delivery of a DNA fragment which expresses GTP cyclohydrolase I.

The present invention also relates to methods of predicting the efficacy of various iNOS-based viral and non-viral constructs for treating the patient by utilizing an in vitro arterial organ culture system to measure various parameters associated with effective iNOS cell transduction.

### 2. BACKGROUND OF THE INVENTION

It is known by those skilled in the art that nitric oxide (NO) is a biologic mediator derived from the amino acid L-arginine. A family of enzymes, known as nitric oxide synthase (NOS), act upon L-arginine to oxidize one of the guanidino nitrogens to nitric oxide while citrulline is formed from the remainder of the L-arginine molecule. Nitric oxide is a very short-lived free radical and is rapidly oxidized to nitrite (NO₂⁻) and nitrate (NO₃⁻) which are measured as the stable inactive end products of nitric oxide formation.

It is well known by those skilled in the art that multiple isoforms of the nitric oxide synthase enzyme exist and that they are generally classified into two broad categories: (1) constitutive and (2) inducible. These classes of NOS enzymes vary considerably in size, amino acid sequence, activity and regulation. For example, cells such as neurons and vascular endothelial cells contain constitutive NOS isoforms while macrophages and vascular smooth muscle cells express an inducible NOS.

It is generally well known that the small amounts of nitric oxide generated by a constitutive NOS appear to act as a messenger molecule by activating soluble guanylate cyclase and, thus, increasing intracellular guanosine, 3′, 5′-cyclic monophosphate (cGMP) and the induction of biological responses that are dependent on cGMP as a secondary messenger. For example, through this mechanism, endothelium derived nitric oxide induces relaxation of vascular smooth muscle and is identified as endothelium derived relaxing factor (EDRF) [Palmer, et al., 1987, Nature 327: 524-526 and Ignarro, et al., 1987, Proc. Natl. Acad. Sci. USA 84: 9265-9269]. Another example includes, but is not limited by, neuronal nitric oxide which acts as a neurotransmitter by activating guanylate cyclase with important functions in the central nervous system and autonomic nervous system (Bredt, et al., 1989, Proc. Natl. Acad. Sci. USA 86: 9030-9033 and Burnett, et al., 1992, Science 257: 401403). It is generally known by those skilled in the art that the sustained production of nitric oxide by the inducible nitric oxide synthase has antimicrobial and antitumor functions. (see Granger, et al., 1989, J. Clin. Invest. 81: 1129-1136 and Hibbs, et al., 1987, Science 235: 473-476, respectively). It is also known by those skilled in the art that when vascular smooth muscle cells are stimulated to express a iNOS enzyme by inflammatory cytokines, the large amounts of nitric oxide released contribute to the vasodilation and hypotension seen in sepsis (Busse and Mulsch, 1990, FEBS Letter 265: 133-136).

Thus, it will be appreciated that nitric oxide has normal physiologic intracellular and extracellular regulatory functions, and in some instances excessive production of nitric oxide can be detrimental. For example, stimulation of inducible nitric oxide synthesis in blood vessels by bacterial endotoxin, such as for example bacterial lipopolysaccharide (LPS), and cytokines that are elevated in sepsis results in excessive dilation of blood vessels and sustained hypotension commonly encountered with septic shock (Kilbourn, et al., 1990, Proc. Natl. Acad. Sci. USA 87: 3629-3632). It is known that overproduction of nitric oxide in lungs stimulated by immune complexes directly damages the lung (Mulligan, et al., 1992, J. Immunol. 148: 3086-3092). Induction of nitric oxide synthase in pancreatic islets impairs insulin secretion and contributes to the onset of juvenile diabetes (Corbett, et al., 1991, J. Biol. Chem. 266: 21351-21354). Production of nitric oxide in joints in immune-mediated arthritis contributes to joint destruction (McCartney, et al., 1993, J. Exp. Med. 178: 749-754).

Several references attempt to link nitric oxide to changes seen in vascular disease. For example, Bucala, et al. (1991, J. Clin. Inv. 87: 432438) disclose that glycosylation products that accumulate in vessel walls during hyperglycemia may quench nitric oxide and reduce nitric oxide availability. Chin, et al. (1992, J. Clin. Inv. 89: 10-18) disclose that oxidized lipoproteins have a similar effect by inactivating nitric oxide. Chester, et al. (1990, Lancet 336: 897-900) disclose that nitric oxide synthesis is reduced in atherosclerotic epicardial arteries in humans. None of these references shed light on therapeutic avenues regarding iNOS-driven gene therapy.

Actions of nitric oxide important to vascular integrity and the prevention of the atherosclerotic lesion include vasodilation (Palmer, et al., 1987, Nature 327: 524-526; Ignarro, et al., 1987, Proc. Natl. Acad. Sci. USA 84: 9265-9269), inhibition of platelet adherence and aggregation (Radomski, et al., 1987, Br. J. Pharmacol. 92: 639-646), inhibition of vascular smooth muscle (Nunokawa, et al., 1992, Biochem. Biophys. Res. Corn. 188:409415) and fibroblast (Werner-Felmayer, et al., 1990, J. Exp. Med. 172: 1599-1607) cellular proliferation. Nitric oxide is normally produced by the vascular endothelium and, because of a very short half-life (t_{½} in seconds), diffuses only to the adjacent smooth muscle where it causes relaxation via the activation of soluble guanylate cyclase (Moncada, et al. 1991, Pharmacol. Rev. 43: 109-142). Nitric oxide released toward the lumen assists in preventing platelet adherence. L-arginine serves as the substrate for nitric oxide formation, and the small amounts of nitric oxide derived from endothelial cells is produced in an ongoing fashion (Palmer. et al., 1987, Nature 327: 524-526; Ignarro, et al., 1987, Proc. Natl. Acad. Sci. USA 84: 9265-9269) by a cNOS, which is located primarily on microsomal and plasma membranes. Agonists such as acetylcholine and bradykinin increase cNOS by activity enhancing calcium/calmodulin binding to the enzyme. The cDNA coding for this enzyme has been cloned from human endothelial cells. (Janssens, et al. 1992, J. Biol. Chem. 267: 14519-14522; Marsden, et al., 1992, FEBS Letters 307: 287-293).

U.S. Pat. No. 5,468,630, issued to Billiar et al. on Nov. 21, 1995, discloses the human iNOS cDNA sequence. The plasmid pHiNOS comprises the human iNOS coding region and was deposited under the terms of the Budapest Treaty on Nov. 20, 1992 and has the ATCC accession number 75358 (pHiNOS) and ATCC accession number 69126 (pHiNOS transformed in E. coli SOLR).

McNamara. et al. (1993, Biochem. Biophys. Res. Comm. 193: 291-296) disclose that addition of L-arginine inhibits mechanically-induced in vivo intimal smooth muscle cell proliferation in rabbit arteries. The authors suggest involvement of endothelial cNOS in the process and provide support for this theory by showing that addition of the cNOS inhibitor N^{G}-nitro-L-arginine methyl arginine reverses the effect of L-arginine in prohibiting intimal hyperplasia. The authors do not address iNOS gene therapy applications. von der Leyen, et al. (1994, FASEB J. 8:A802 (#4651), Abstract) recite use of a Sendai virus-mediated transfection protocol for transfer of cNOS into a rat carotid artery. The abstract does not teach or suggest use of iNOS in gene therapy applications.

Takashita, et al. (1994, J. Clin. Invest. 93:652-661) disclose liposome-mediated in vitro and in vivo reporter gene transfer into porcine arterial smooth muscle cells. The authors suggest a positive correlation between expression of the transferred reporter gene and proliferation of intimal smooth muscle cells in response to mechanical injury. The authors report only in vitro and in vivo porcine arterial cell expression of reporter genes encoding luciferase and β-Gal.

U.S. Pat. No. 5,428,070. issued to Cooke, et al., discloses treatment of vascular occlusive disease by administering L-arginine in an attempt to increase ecNOS expression. No basis for gene therapy applications are taught or suggested in this disclosure nor is any suggestion forwarded to increase local vascular iNOS concentrations, not to mention by means of gene therapy.

Despite iNOS related systemic toxicity seen in various tissues. it would be advantageous to target local cell populations with a DNA sequence encoding iNOS or a biologically active fragment or derivative; such a gene therapy treatment will promote prophylactic and/or therapeutic actions in regard to diseases or disorders including but not necessarily limited to vascular occlusive disease, tumor cell growth associated with cancer, numerous microbial infections, treating tissue injury, and to promote wound healing.

### 3. SUMMARY OF THE INVENTION

The present invention has met the hereinbefore described needs. The present invention provides for use of a DNA fragment expressing inducible nitric oxide synthase (iNOS) or a biologically active fragment or derivative thereof in gene therapy techniques to treat any number of maladies effected by nitric oxide, including but not solely limited to (1) vascular occlusive disease associated with atherosclerosis; (2) resisting vascular conduit occlusion due to thrombosis, intimal hyperplasia, or atherosclerosis; (3) treatment of accelerated vascular occlusive disease associated with diabetes mellitus which results in a high incidence of myocardial infarction, renal failure, stroke, blindness and limb loss at an early age; (4) treatment of cancer, specifically as an antitumor agent by increasing local nitric oxide concentrations in and around the tumor(s); (5) treatment of various microbial infections; (6) treatment of various tissue injuries, including but not limited to damage to the liver; and (7) promotion of wound healing.

The present invention provides for optimization of the local effect imparted by means of expressing iNOS within target vascular cells. This embodiment relates to in vitro or in situ-based target vascular cell delivery of a DNA fragment which expresses iNOS and a DNA fragment which expresses GTP cyclohydrolase I (GTPCH). DNA fragments expressing these genes may be delivered as part of the same recombinant vector or on separate recombinant vectors, using any techniques disclosed within this specification or known to the skilled artisan.

In regard to treatments (1), (2) and (3) disclosed in the previous paragraph (herein referred to as vascular diseases or vascular disorders), local tissue specific expression of iNOS in targeted cells will result in the production of effective amounts of nitric oxide in the area of expression, so as to promote maximal local vasodilation, resist local thrombosis and potentially retard local smooth muscle cell proliferation, all of which may prevent the atherosclerotic disease process. It will be understood to one of ordinary skill in the art that any nucleic acid sequence encoding an inducible form of NOS, preferably human iNOS, regardless of the tissue source, is a candidate for utilization in, for example, gene therapy of vascular occlusive complications associated with atherosclerosis, vascular bypass, and diabetes derived vascular disease at sites of anastomosis. It will be further understood by the skilled artisan that any nucleic acid sequence which encodes a biologically active form of iNOS, preferably a human form of iNOS, including but not limited to a genomic or cDNA sequence or a fragment thereof which encodes a biologically active protein fragment or derivative, may be utilized in the present invention.

The present invention discloses treatment of vascular diseases or vascular disorders by increasing local iNOS activity, and thus nitric oxide concentrations, through targeting of mammalian cell populations which comprise the luminal lining of the arterial vessel, namely endothelial cells and vascular smooth muscle cells. More specifically, the target mammalian cells may be, but are not necessarily limited to: (1) in vitro cultured endothelial cells and (2) in vitro cultured vascular smooth muscle cells. These cells may be transduced with a DNA sequence encoding iNOS or a biologically active fragment or derivative thereof and may be subsequently utilized to repopulate arterial vessels of the patient. It is also within the scope of this invention to use iNOS-expressing endothelial cells, vascular smooth muscle cells or a combination of both to repopulate a diseased vessel or to seed a synthetic or autologous graft.

It will be preferred to utilize endothelial and/or smooth muscle cells obtained from the patient, which may be isolated and cultured by any number of methods known to one of ordinary skill in the art. A direct source of these vascular cells may be obtained, for example, by harvesting a portion of a saphenous vein or any other accessible vein or artery from the patient. This mode of obtaining target cell source material for in vitro culture prior to iNOS infection or transfection procedures will be especially useful in seeding a synthetic or autologous graft for transfer to the patient.

In another embodiment of the present invention, endothelial cells, vascular smooth muscle cells or a combination of both are targeted for in situ infection or transfection with a DNA sequence encoding iNOS or a biologically active fragment or derivative thereof so as to promote increased local iNOS expression within selected segments of arterial vessels.

It will be understood by the skilled artisan that similar procedures may be utilized for in vitro transfection or infection of endothelial cells and vascular smooth muscle cells. Both endothelial cells and vascular smooth muscle cells may be infected simultaneously through an in situ procedure, exemplified but not limited to the procedures outlined in the appended Example Sections.

It will also be understood that the skilled artisan will have access to numerous endovascular surgical techniques to direct in situ or in vitro based applications of the present invention. These techniques may be used within an arterial vessel segment showing adequate circulation or may also be used subsequent to clearing an arterial vessel segment of an occlusion or stenosis. It will be known to the skilled vascular surgeon that various endovascular surgical techniques are available, depending upon the severity of the occlusion and location of arterial vessel target for treatment. For a review of endovascular alternatives, see generally Ahn, 1993, “Endovascular Surgery,” in Vascular Surgery: A Comprehensive Review, Ed. W. S. Moore, W. B. Saunders & Co., Philadelphia). Endovascular surgical procedures will utilize catheter devices which include but are not limited to balloon angioplasty, intravascular stents, laser-assisted balloon angioplasty, double balloon catheterization, mechanical endarterectomy and vascular endoscopy. It will also be understood that one or more endovascular procedures available to the skilled vascular surgeon may be utilized to prepare the diseased vessel for iNOS-based gene therapy as well as to deliver the DNA sequence encoding iNOS to the conduit area targeted for treatment.

It will also be understood by the skilled artisan that a combination of strategies disclosed further within this specification may be utilized in conjunction with surgical vascular bypass procedures to promote a gene therapy based increase in local iNOS expression at sites of surgical repair or within a synthetic graft.

In a particular embodiment regarding in vitro as well as in situ-based targeting of vascular cells, a DNA sequence encoding iNOS or a biologically active fragment thereof will be ligated to a viral vector in preparation for tissue specific delivery and expression. Virus vector systems utilized in the present invention include, but are not limited to (a) retroviral vectors, including but not limited to vectors derived from the Moloney murine leukemia virus (MoMLV) genome; (b) adeno-associated vectors; (c) adenovirus vectors; (d) herpes simplex virus vectors; (e) SV40 vectors; (f) polyoma virus vectors; (g) papilloma virus vectors; (h) picornavirus vectors; and (i) vaccinia virus vectors.

Additional strategies which the skilled artisan may utilize alone or in combination with viral vectors in targeting endothelial cells, vascular smooth muscle cells or a combination thereof for gene therapy of vascular diseases include but are not limited to (a) liposome-mediated transformation; (b) calcium phosphate [Ca₃(PO₄)₂] mediated cell transfection; (c) in vitro transfection of target cells by electroporation; (d) DEAE-dextran mediated cell transfection, the in vitro transfected cells then utilized to repopulate the mammalian host: (e) polybrene mediated delivery; (f) protoplast fusion; (g) microinjection: (h) polylysine mediated transformation: and (i) direct injection of naked DNA. The genetically transformed cells generated by any of these strategies are then utilized to repopulate the mammalian host.

In a particular embodiment regarding the in vitro based treatment of vascular diseases, a recombinant viral vector comprising a DNA sequence encoding iNOS or a biologically active fragment or derivative utilized to infect mammalian endothelial cells, vascular smooth muscle cells or a combination of both for repopulation of arterial vessels is a recombinant retroviral vector. The respective iNOS DNA sequence is ligated within the retroviral vector to form a retroviral-iNOS recombinant construct.

In a particular embodiment regarding the in situ based treatment of vascular diseases, a recombinant viral vector comprising a DNA sequence encoding iNOS or a biologically active fragment or derivative targeted for direct delivery to vascular cells is a recombinant retroviral vector. The respective iNOS DNA sequence is ligated within the retroviral vector to form a retroviral-iNOS recombinant construct.

In a preferred embodiment regarding the treatment of vascular diseases, the iNOS sequence subcloned into an appropriate retroviral vector is a human iNOS sequence.

In a further preferred embodiment regarding use of a retroviral vector in gene therapy of vascular diseases. the recombinant retroviral vector is a MoMLV-iNOS construct. This iNOS containing retroviral construct comprises a human DNA sequence encoding iNOS or a biologically active fragment or derivative thereof.

A preferred embodiment regarding use of a retroviral vector in gene therapy of vascular diseases, the MoMLV-iNOS construction is DFG-iNOS-Neo as depicted in FIG. 6 and FIG. 8. The DFG-iNOS-Neo construct is preferred for in vitro infection of endothelial cells or vascular smooth muscle cells.

Another embodiment regarding use of a retroviral vector in gene therapy of vascular diseases, the MoMLV-iNOS construct is MFG-iNOS as depicted in FIG. 6 and FIG. 7.

Any of the hereinbefore disclosed retroviral-iNOS recombinant constructs are then transferred into a standard retroviral packaging cell line. The recovered recombinant viral particles are then used to infect cultured endothelial cells or vascular smooth muscle cells in vitro. Treatment of vascular diseases is based further on transferring in vitro transduced or infected endothelial cells, vascular smooth muscle cells or a combination of both to specific segments of diseased arteries within the patient. Any of the following endovascular surgical procedures may be useful to the skilled artisan, including but are not limited to balloon angioplasty, intravascular stents, laser-assisted balloon angioplasty, double balloon catheterization, mechanical endarterectomy and vascular endoscopy.

A preferred recombinant viral vector for practicing a portion of the present invention is an adenovirus vector. The human iNOS cDNA is subcloned into an adenovirus vector to generate a recombinant adenovirus-iNOS based construct. An adenovirus-iNOS based construct of the present invention will be useful in both in situ and in vitro based applications.

An especially preferred recombinant adenovirus-iNOS based construct of the present invention is Ad-iNOS. A recombinant AdiNOS vector is preferred for in situ gene therapy applications. The AdiNOS construct used to exemplify a portion of the present invention comprises a full iNOS cDNA was inserted along with a CMV enhancer/promoter complex. This AdiNOS construct constitutively expresses the E1 gene product and are therefore able to package infectious adenoviral particles from E1 deleted constructs. Following transfection, intracellular recombination occurs to generate the full-length adenoviral genome containing the iNOS cDNA.

In vitro viral-mediated infection or vector-mediated transfection of endothelial cells or vascular smooth muscle cells with a DNA sequence encoding iNOS or a biologically active fragment thereof may be accomplished by numerous non-biologic and/or biologic carriers other than the hereinbefore mentioned retroviral and adenovirus vectors. Therefore, any non-biologic and/or biologic carrier possessing the ability to deliver an iNOS encoding DNA sequence to the local target such that iNOS is expressed at therapeutic or prophylactic levels may be utilized to practice the present invention.

For example, another embodiment of the invention involves a DNA sequence encoding iNOS or a biologically active fragment thereof which may be subcloned into an adeno-associated viral vector (AAV). As with an Ad-iNOS construct, the recombinant AAV-iNOS construct can be utilized to directly infect in vitro cultured endothelial cells, vascular smooth muscle cells or a combination thereof, may be delivered directly to the target vascular cells by in situ administration, or alternatively, can be delivered to the target cells through the association with liposome microcapsules in either an in vitro or in situ-based application.

In a further embodiment regarding the use of liposome-mediated techniques to deliver recombinant iNOS constructs to treat vascular diseases, a viral or non-viral vector comprising a DNA sequence encoding iNOS is delivered to the target cell by lipofectamine transfection. For example, a DNA sequence encoding iNOS or a biologically active fragment thereof is subcloned into a DNA plasmid vector such that iNOS is expressed subsequent to transfection of the target cell. Such non-viral based mammalian vectors include, but are not solely limited to, a plasmid DNA mammalian expression vector. Any eukaryotic promoter and/or enhancer sequence available to the skilled artisan which is known to up-regulate expression of iNOS may be used in mammalian expression vector constructs. including but not limited to a cytomegalovirus (CMV) promoter, a Rous Sarcoma (RSV) promoter, a Murine Leukemia (MLV) promoter, a herpes simplex virus (HSV) promoter, such as HSV-tk, a β-actin promoter, as well as any additional tissue specific or signal specific regulatory sequence that induces expression in the target cell or tissue of interest. A signal specific promoter fragment includes but is not limited to a promoter fragment responsive to TNF.

In one such embodiment, a DNA sequence encoding human iNOS is subcloned into the DNA plasmid expression vector. pCIS (Genentech), resulting in pCIS-iNOS. pCIS is a standard mammalian expression vector, containing an antibiotic resistance gene for propagation in E. coli and a CMV promoter active in mammalian cells. Such a construct, which may be constructed by one of ordinary skill with components available from numerous sources, will drive expression of an iNOS DNA fragment ligated downstream of the CMV promoter subsequent to transfection of the target cell. More specifically, a NotI/XhoI restriction fragment containing the human iNOS coding region is generated and isolated from pHiNOS (pHiNOS is deposited with the ATCC with accession number 75358) and ligated into NotI/XhoI digested pCIS. Alternatively, the isolated human iNOS sequence may be fused to any portion of the wild type human iNOS promoter sequence such that expression of human iNOS can be induced within the target cell.

It will become evident to one of ordinary skill in the art upon review of this specification that any of the viral or non-viral recombinant iNOS constructs hereinbefore described for use in infecting or transfecting in vitro cultured endothelial cells, vascular smooth muscle cells or a combination thereof may be used to infect or transfect a target cell in situ. For example, an endovascular procedure available to the skilled vascular surgeon may be utilized to dilate an occluded segment of diseased arterial vessel so as to reestablish the arterial lumen. The dilated segment is then segregated from the remainder of the arterial vessel. A viral or non-viral based recombinant iNOS construct may be selectively delivered through the catheter to the angioplasty site so as to promote in situ transfection or infection of endothelial and/or vascular smooth muscle cells with concomitant local increases in iNOS expression within the diseased vessel segment.

Another embodiment of the present invention relates optimizing the local vascular cell iNOS effect by the concomitant in situ delivery of a DNA fragment expressing GTP cyclohydrolase I.

A preferred embodiment of a tandem delivery DNA fragments expressing iNOS and GTP cyclohydrolase I provides for use of a recombinant adenovirus viral vector to deliver the recombinant viral vector or vectors to the local arterial segment within the patient.

The present invention also relates to a method of determining the precise efficacy of a transgenic construction upon in situ infection of a diseased human artery.

One embodiment of determining the precise efficacy of a transgenic construction upon in situ infection of a diseased human artery involves obtaining diseased human arteries by methods known the to skilled vascular surgeon, placing the excised arteries in culture, infecting the cultured arteries with the transgenic construct of interest, and measuring various parameters such as intimal hyperplasia, gene expression, and generation of various metabolites. Exemplified sources for organ culture and testing are human coronary arteries obtained from the extirpated hearts of patients undergoing cardiac transplantation and human peripheral arteries obtained from patients undergoing limb amputation for vascular occlusive disease. Such diseased arteries will be readily available to the skilled vascular surgeon due to the routine performance of amputations. Additionally, fresh cadavers or organ donors may also be a potential source for using diseased human arteries as disclosed in this specification.

A second embodiment in determining the precise efficacy of a transgenic construction upon in situ infection of a diseased human artery involves obtaining porcine artery or artery from another experimental mammalian system either obtained in a diseased state or subjected to injury subsequent to excision from the animal. These arteries are also retrieved by methods known to the to skilled vascular surgeon, followed by placing the excised arteries in culture, infecting the cultured arteries with the gene therapy transgenic construct of interest. and measuring various parameters such a intimal hyperplasia and various metabolite generation, and levels of transgene expression or native gene expression.

A preferred embodiment of determining the precise efficacy of a transgenic construction upon in situ infection of a diseased human artery involves infecting either a diseased human artery or diseased or normal porcine artery with an iNOS-based construct, whereby post infection measurements include, but are not limited to NO₂⁻+NO₃⁻ production, cGMP production and changes in medial thickness of diseased arteries in response to infection of a transgenic iNOS construct. This in vitro based system will also be utilized to assess the level of transgene and endogenous gene expression by RT-PCT analysis, Northern blot analysis. Western blot analysis or enzyme assays.

The in vitro culture and use of a human or another mammalian arterial segment to determine efficacy of transgene constructs include a DNA fragment encoding a full length or biologically active fragment which expresses a protein that supplies cofactors related to iNOS metabolism, including but not limited to GTP cyclohydrolase I as well as genes expressing proteins that interrupt the cell cycle, including but not limited to p21, p53 or Rb.

The present invention also discloses methods of human iNOS-directed gene therapy to promote antitumor effects in cancer patients. Such a human iNOS-directed gene therapy will provide a local increase in nitric oxide concentration within the area of the tumor to be treated, thus promoting antitumor activity without systemic increases in nitric oxide levels. As disclosed for iNOS-mediated treatment, a human derived DNA sequence encoding iNOS or a biologically active fragment or derivative thereof is preferred.

The isolated human iNOS DNA sequence may be manipulated and delivered to the target cell in vitro by transduction utilizing any of the viral and non-viral methods discussed in Section 5.2.1. The in vitro transduced target cells are then introduced into the patient so as to promote local iNOS expression at the tumor site. Therefore, it will be understood that any human iNOS DNA sequence encoding a biologically active fragment or derivative thereof, regardless of tissue source. is a candidate for antitumor treatments.

In one embodiment regarding cancer gene therapy, the patient is intravenously injected with in vitro transduced target cells, including but not limited to tumor infiltrating lymphocytes or cultured tumor cells harvested from the patient.

In a preferred method of delivering a human iNOS sequence to the target cell of interest, a recombinant retroviral vector comprising a DNA sequence encoding iNOS or a biologically active fragment thereof is utilized to infect tumor infiltrating lymphocytes. These infected tumor infiltrating lymphocytes are then reintroduced into the patient to promote local expression of iNOS at the tumor site.

In a preferred embodiment regarding gene therapy of cancer, DFG-iNOS-Neo (FIG. 8) is utilized to infect tumor infiltrating lymphocytes or cultured tumor cells harvested from the patient. Neomycin resistant cells are selected, followed by localization of these iNOS expressing cells to the region within and surrounding the active tumor.

The present invention also relates to in situ iNOS-based treatment of hepatocellular carcinomas, including malignant epithelial neoplasms of the liver, as well as liver metastases. A preferred method of treating liver cancer in situ involves an intravenous, systemic administration of an AdiNOS construct, which will result in an approximately 95% targeting to the liver. These treatments will be available for use alone or in tandem with one or more of recognized systemic or intrahepatic arterial chemotherapy regimes, cytokine immunotherapy (especially including TNF-α) procedures, and radiation therapy, all useful in treating various stages of hepatic tumors.

In addition to the hereinbefore described use of viral vectors to infect target cells, any known non-viral vector described in this specification may be utilized to promote antitumor activity.

Another embodiment of the present invention relates optimizing the antitumor effect generated by local iNOS expression by the concomitant in situ delivery of a DNA fragment expressing GTP cyclohydrolase I.

A preferred embodiment of a tandem delivery DNA fragments expressing iNOS and GTP cyclohydrolase I provides for use of a recombinant adenovirus viral vector or vectors to direct delivery to the liver to maximize in situ treatment of hepatocellular carcinomas.

The human iNOS DNA sequences of the present invention may also be utilized in treating microbial infections. Specifically, iNOS-driven antimicrobial therapy will be utilized to treat microbes known to be susceptible to increased concentrations of nitric oxide. For example, nitric oxide is known to be a cytotoxic effector molecule against mycobacteria, helminths, fungi, protozoa and DNA viruses. Therefore, the present invention discloses methods of increasing concentrations of nitric oxide locally at the site of infection by targeting the infected cell or tissue type with a DNA sequence encoding iNOS activity, preferably human iNOS. capable of being expressed at a therapeutic level and duration so as to surmount the disease.

In a preferred embodiment of utilizing iNOS-driven antimicrobial therapy, the target cell type is human hepatocytes infected with the sporozoa Plasmodium, the causative agent of malaria. Human malaria is caused by one of four species of Plasmodium: P. falciparum, P. malariae, P. vivax and P. ovale.

Another preferred embodiment for utilizing iNOS-driven antimicrobial therapy is targeting human hepatocytes with AdiNOS. Again, the liver is directly targeted by systemically delivering a recombinant adenovirus which expresses iNOS. Therefore, a preferred method of in situ treatment of a microbial infection involving the liver involves an intravenous, systemic administration of an AdiNOS construct, which will result in an approximately 95% targeting of a recombinant AdiNOS vector to the liver.

Another preferred embodiment of treating malaria via iNOS-antimicrobial therapy, the iNOS-vector is delivered via liposome mediated transformation of the target hepatocytes. The liposomes are modified by insertion of an hepatocyte specific asialoprotein into the liposome membrane prior to administration to the patient.

Additionally, a preferred method of treating malaria in the present invention involves targeting human hepatocytes with AdiNOS. Again, the liver is directly targeted by systemically delivering a recombinant adenovirus which expresses iNOS. Therefore, a preferred method of in situ treatment of malaria will also include an intravenous, systemic administration of an AdiNOS construct. which will result in an approximately 95% targeting of a recombinant AdiNOS vector to the diseased tissue.

Another embodiment of utilizing iNOS-vectors in antimicrobial therapy involves treatment of lung borne microbial infections, including but not limited to tuberculosis and leprosy.

A preferred treatment of tuberculosis by iNOS-antimicrobial therapy involves targeting an iNOS vector to the target tissue by viral mediated transformation of cells within the target tissue.

A preferred method of treating tuberculosis by iNOS-antimicrobial therapy is adenovirus-mediated delivery to the site of infection.

Another preferred method of treating tuberculosis by iNOS-driven biologic therapy is retroviral mediated delivery, as discussed in Section 5.2.1. iNOS-based vectors disclosed in Sections 5.2.1 and 5.2.2 may also be utilized in retroviral-mediated delivery techniques to treat tuberculosis.

With the aid of this specification, it would be within the realm of the artisan of ordinary skill to construct an iNOS vector compatible with the delivery system of choice for use in treating tuberculosis.

A preferred method of administering an iNOS-infected retrovirus within infected regions of lung tissue is inhalatory administration, in the form of an aerosol mist.

Another embodiment of the invention relates to treatment of Mycobacterium leprae, the causative agent of leprosy. The preferred mode of treating leprosy by gene therapy entails retroviral-mediated transduction of target tissue cell types by inhalatory administration.

The present invention also relates to gene therapy applications to promote wound healing. Nitric oxide has been shown to promote angiogenesis. For example, a mouse deleted for the iNOS gene subjected to wounding shows a propensity for faster healing when administered an iNOS source compared to a control wherein a source of iNOS is not supplied. Therefore, a preferred embodiment of the present invention to promote wound healing relates to direct application of iNOS or iNOS/GTP cyclohyrdolase I. Any pharmaceutically effective composition comprising an iNOS source may be applied directly to the wound. The scope of iNOS induced angiogenesis includes treating a primary wound union such as will be evident with an internal suture such as a bowel suture. The scope of iNOS induced angiogenesis also includes treating a second wound union whereby-more extensive cell and tissue loss has occurred, such as an inflammatory ulceration, an infarction, abscess formation and large defect surface wounds. A preferred method of treating non-healing wounds with iNOS is to promote optimal infection of the wound area with a recombinant iNOS vector incorporated into a pharmaceutically effective carrier. It will also be feasible to introduce an iNOS or iNOS/GTP cyclohyrdolase I construct via a biobalistic device for surface wounds. A further preference is the application of AdiNOS to the non-healing wound, with an especially preferred method involving application of an AdiNOS composition to a non-healing leg ulcer to promote on site angiogenesis.

As related to targeting the liver to treat liver cancer and microbial infections of the liver, the present invention also relates to treatment of various liver injuries. Hepatotoxins which may provoke injury to the liver which are amenable to iNOS gene therapy include but are not limited to acetaminophen. isoniazid, α-methyldopa, chlorpromazine, methotrexate, halothane and tetracycline. Applications of an iNOS expressing transgene construct will also be useful in overcoming TNF-α toxicity sometimes associated with liver injury as seen in inflammation associated with hepatitis. Therefore, a preferred method of in situ treatment of liver injuries which involves an intravenous, systemic administration of an AdiNOS construct, which will result in an approximately 95 % targeting of a recombinant AdiNOS vector to the liver and in turn an optimal therapeutic effect.

The present invention relates to optimizing the local vascular cell iNOS effect by the concomitant in situ delivery of a DNA fragment expressing GTP cyclohydrolase I. This method may be incorporated with treatment involving cancer, microbial infections, tissue injuries, and promotion of wound healing.

A preferred embodiment of a tandem delivery DNA fragments expressing iNOS and GTP cyclohydrolase I provides for the treatment of cancer, microbial infections, tissue injuries, and promotion of wound healing by utilizing a recombinant adenovirus viral vector or vectors for tandem in situ delivery of DNA fragments expressing iNOS and GTP cyclohydrolase I.

It is an object of this invention to provide vascular gene therapy to provide prophylactic and therapeutic relief from vascular diseases including but not limited to vascular occlusive diseases associated with atherosclerosis, vascular bypass. and associated with diabetes by providing transfected endothelial cells, vascular smooth muscle cells or a combination of both which express iNOS or a biologically active fragment thereof to a patient's diseased blood vessel, a vascular conduit, or blood vessel partly or totally denuded of its endothelial lining.

It is an object of this invention to provide for in situ GTP cyclohydrolase I expression in neighboring cells targeted for iNOS infection and expression so as to optimize the therapeutic effect of iNOS in treating the disease or disorder of choice.

It is an object of this invention to provide a method of determining the precise efficacy of a transgenic construction upon in situ infection of a diseased human artery.

It is an object of this invention to provide therapeutic treatment of tumor growth by utilizing iNOS-driven gene therapy techniques to increase local nitric oxide concentrations so as to inhibit tumor growth.

It is an object of this invention to provide therapeutic treatment of tumor growth by utilizing iNOS-driven gene therapy techniques to increase local nitric oxide concentrations so as to inhibit tissue injuries.

It is an object of this invention to provide therapeutic treatment of tumor growth by utilizing iNOS-driven gene therapy techniques to increase local nitric oxide concentrations so as to promote wound healing.

It is an object of this invention to provide therapeutic relief from various microbial infections susceptible to attack by utilizing iNOS-driven gene therapy techniques to increase local concentration of nitric oxide at or around the site of infection, especially the various pulmonary and hepatic infections described in this specification.

These and other objects of the invention will be more fully understood from the following description of the invention, the figures, the sequence listing and the claims appended hereto.

### 3.1. DEFINITIONS

The terms listed below. as used herein, will have the meaning indicated.

| | |
|---|---|
| mRNA | messenger RNA |
| DNA | deoxyribonucleic acid |
| cDNA | complementary deoxyribonucleic acid |
| NO | nitric oxide |
| NOS | nitric oxide synthase |
| ecNOS | endothelial constitutive nitric oxide synthase |
| | (type 3 NOS) |
| iNOS | inducible nitric oxide synthase |
| | (type 3 NOS) |
| EDRF | endothelium derived relaxing factor |
| LPS | lipopolysaccharide |
| CMV | cytomegalovirus |
| Ad | adenovirus |
| AAV | adeno-associated virus |
| IRES | internal ribosome entry site |
| PTFE | polytetrafluoroethylene |
| SPAEC | sheep pulmonary artery endothelial cells |
| RSMC | rat pulmonary artery smooth muscle cells |
| cGMP | cyclic GMP |
| BH₂ | dihydrobiopterin |
| BH₄ | tetrahydrobiopterin |
| GTPCH | GTP cyclohydrolase I |

As used herein, the term “patient” includes members of the animal kingdom including but not limited to human beings.

As used herein, the term “mammalian host” includes mammals, including but not limited to human beings.

As used herein, the term “biologically active fragment or derivative thereof” includes any iNOS protein fragment possessing similar biological activity as wild type iNOS, or a derivative such as an iNOS substitution, addition and/or deletion mutant which maintains similar biological activity as wild type iNOS. One of ordinary skill in the art may use the present specification to generate such changes in the wild type iNOS DNA sequence so as to express variants of wild type iNOS which retain the biological activity necessary to be useful in the presently disclosed gene therapy applications.

As used herein, the term “in vitro” is interchangeable with the term “ex vivo”, thus denoting a manipulation of the target cell outside of the patient prior to reintroduction and generation of a therapeutic response.

As used herein, the term “in situ” is interchangeable with the term “in vivo”, thus denoting a manipulation of the target cell within of the patient, followed by generation of a therapeutic response.

As used herein, the term “vascular surgeon” may refer to cardiovascular surgeons, invasive cardiologists, interventional radiologist, and specialists in vascular surgical techniques.

### 4. BRIEF DESCRIPTION OF THE FIGURES

The file of this patent contains at least one drawing executed in color. Copies of this patent with color drawing(s) will be provided by the Patent and Trademark Office upon request and payment of the necessary fee.

FIGS. 1A-G show the cDNA sense sequence (top line of each horizontal row; SEQ ID NO: 1) and the amino acid sequence of amino acids 1-1153 (bottom line of each horizontal row; SEQ ID NOS: 1 and 2) for the cDNA clone for human hepatocyte inducible nitric oxide synthase.

FIG. 2 shows a Northern blot of a mouse macrophage NOS cDNA cross-hybridizing to human hepatocyte (HC) nitric oxide synthase mRNA.

FIG. 3 shows a Northern blot of induced nitric oxide synthase mRNA isolated from three separate human liver samples using mouse macrophage cDNA.

FIG. 4 shows a Northern blot of poly A mRNA purified from 2 separate human liver samples used in the construction of the cDNA library for isolation of the cDNA clone for the human hepatocyte inducible nitric oxide synthase.

FIG. 5 shows a Northern blot using cDNA isolated from human hepatocytes that sets forth the time course of induction of human nitric oxide synthase mRNA following cytokine and LPS stimulation.

FIG. 6 shows the MFG-iNOS and DFG-iNOS-Neo recombinant retroviral vectors utilized to exemplify the gene therapy applications to treat diseases or disorders disclosed throughout this specification as well as control recombinant retroviral vectors MFGlacZ and BaglacZ. MFGlacZ was previously constructed and does not include the Neo selectable marker. BaglacZ is a retroviral vector carrying both lacZ and Neo. Neo^{r} encodes resistance to neomycin; the IRES fragment allows translation of a polycistronic mRNA; LTR are long terminal repeats of the MoMLV genome; iNOS is the cDNA encoding human hepatocyte iNOS.

FIG. 7 shows detailed methods utilized to construct MFG-iNOS, a recombinant retroviral vector utilized to exemplify various gene therapy applications disclosed throughout this specification.

FIG. 8 shows detailed methods utilized to construct DFG-iNOS-Neo, a recombinant retroviral vector utilized to exemplify various gene therapy applications disclosed throughout this specification.

FIG. 9 shows nitrite production in cultured porcine endothelial cells infected with MFG-iNOS, MFG-lacZ and uninfected cells in the absence and presence of the iNOS inhibitor, N^{G}-monomethylarginine.

FIG. 10 shows nitrite production in cultured porcine endothelial cells infected with DFG-iNOS-Neo, MFG-lacZ and uninfected cells in the absence and presence of the iNOS inhibitor, N^{G}-monomethylarginine.

FIG. 11 shows nitrite production in vascular smooth muscle cells after lipofectamine transfection of pCIS-iNOS in the absence and presence of N^{G}-monomethylarginine, pSV-lacZ, and a plasmid-less control with or without the addition of liposomes.

FIG. 12 shows Northern blot analysis for human iNOS mRNA in SPAEC, SPAEC-BaglacZ, and SPAEC-DFGiNOS cells. A 7.5 kb iNOS signal was detected in total RNA from SPAEC-DFGiNOS while no signal could be detected in either uninfected SPAEC or SPAEC-BaglacZ. The endogenous human hepatocyte iNOS mRNA in cytokine stimulated human hepatocytes (Hum HC+CM, 6 h) measures 4.5 kb in size. SPAEC-DFGiNOS is SPAEC infected with DFG-iNOS-Neo.

FIG. 13 shows Western blot analysis for human iNOS protein expression in SPAEC, SPAECBaglacZ, and SPAEC-DFGiNOS cytosol preparations. A sample of cytokine stimulated human hepatocyte cytosol served as the positive control for the 131 kD iNOS protein. SPAEC-DFGiNOS is SPAEC infected with DFG-iNOS-Neo.

FIG. 14 shows comparison of NO₂⁻ production by uninfected SPAEC, SPAEC-BaglacZ, and SPAEC-DFGiNOS. Each bar represents the means +SD (n=3 for each group, experiments were repeated 3 or more times with comparable results). L-NMA was added at 0.5 mM concentrations while BH₄ was supplemented at 100 μM. (*p<0.01 vs. uninfected+L-NMA, +BH₄, and BaglacZ+L-NMA, +BH₄, by ANOVA). SPAEC-DFGiNOS is SPAEC infected with DFG-iNOS-Neo.

FIG. 15A shows Northern blot analysis for ecNOS mRNA in total RNA samples from SPAEC, SPAEC-DFGiNOS grown in the presence of 0.5 mM L-NMA, SPAEC-DFGiNOS grown without iNOS inhibition for greater than 7 days, SPAEC exposed to 1 mM SNAP for 6 h, and SPAEC exposed to 1 mM SNAP for 6 h. Human hepatocytes (HC) stimulated with a mixture of cytokines serves as a negative control.

FIG. 15B shows Western blot analysis for ecNOS protein expression in whole cell preparations of SPAEC, SPAEC-DFGiNOS cultured in L-NMA (SPAEC-DFGiNOS+NMA), and SPAEC-DFGiNOS permitted to synthesize NO for >7 days (SPAEC-DFGiNOS). SPAEC-DFGiNOS is SPAEC infected with DFG-iNOS to show that iNOS transfer does not reduce native ecNOS expression.

FIG. 16A shows NO2⁻- production as measured by the Griess reaction in uninfected, BaglacZ. and RSCM-DFGiNOS. Each bar represents the mean+SD (n=3, each experiment repeated three times). iNOS activity was measured +L-NMA (0.5 mM), +BH₄ (100 uM). (*p<0.01 vs. uninfected and BaglacZ cells by ANOVA). RSCM-DFGiNOS is SPAEC infected with DFG-iNOS.

FIG. 16B shows Northern blot analysis for human iNOS mRNA in RSMC, RSMC-BaglacZ, and RSCM-DFGiNOS cells. A 7.5 kb iNOS signal was detected in total RNA from RSCM-DFGiNOS only. The endogenous human hepatocyte iNOS mRNA from cytokine stimulated human hepatocytes (Hum HC+CM, 6 h) measures 4.5 kb.

FIG. 17 shows RT-PCR amplification for iNOS and Neo expression in MFGlacZ and DFG-iNOS infected balloon catheter-injured porcine femoral arterial segments 9 days after infection with retroviral vectors. The iNOS PCR product measures 316 bp while the Neo product measures 728 bp. RT-PCR was also performed for B-actin expression as a control for the first strand cDNA synthesis reaction and PCR amplification (PCR product 652 bp).

FIG. 18 shows (A) NO₂⁻+NO₃⁻ production, (B) cGMP production, and (C) vascular smooth muscle thickness in porcine femoral arteries uninfected or infected with DFG-iNOS or MFGlacZ either exposed to arterial injury.

FIG. 19 shows (A) NO₂⁻+NO₃⁻ production, (B) cGMP production, and (C) vascular smooth muscle thickness in human coronary and tibial arteries uninfected or infected with DFG-iNOS or MFGlacZ either exposed to arterial injury.

FIG. 20 (upper panel) shows X-gal staining for β-galactosidase activity in MFGlacZ infected porcine arterial segments. The arrows indicate positively staining (blue) cells located in superficial layers as viewed from the lumenal surface.

FIG. 20 (lower panel) shows immunolocalization for iNOS protein in DFGiNOS infected porcine arterial segments in cross section. The arrows indicate positively staining cells which are isolated to the thin neointimal layer. No such staining was evident in the media.

FIG. 21A (10×magnification) and FIG. 21B (20×magnification) show a cross section micrograph of porcine arterial vessels infected in situ with either AdiNOS (=AdNOS) or the control plasmid AdlacZ 14 days post infection.

FIG. 22 shows Northern blot analysis for human GTPCH mRNA in 3T3-DFGiNOS and RSMC cells transfected with either pIEP-lacZ or pCIS-GTPCH. The 900 bp recombinant GTPCH signal was detected only in cells transfected with pCIS-GTPCH. Endogenous GTPCH mRNA signals are absent from all the cells and would measure over 3 kb in size, comparable to the signal detected in the human hepatocyte control (lane 1). 18s rRNA shows equivalent RNA loading. This experiment is representative of 3 separate experiments.

FIG. 23 shows comparison of iNOS enzymatic activity as measured by NO₂⁻production in 3T3-iNOS transfected with pIEP-lacZ or pCIS-GTPCH. Each bar represents the means ±SEM (n=3 for each group, experiments were repeated 3 or more times with comparable results). L-NMA was added at 1 mM concentrations, BH₄ at 100 μM, and MTX at 12.5 μM (the lowest effective dose to block dihydrofolate reductase activity with minimal cytotoxicity). (*p<0.01 vs. pIEP-lacZ)

FIG. 24 shows the effect of BH₄ synthesis by 3T3 cells on the iNOS activity in cocultured 3T3-iNOS cells. 3T3-iNOS cells cultured with control transfected 3T3 cells showed no detectable NO₂ production. 3T3-iNOS cells cultured with pCIS-GTPCH transfected 3T3 cells recovered maximal iNOS activity, equivalent to that obtained with exogenous BH₄ administration. (p<0.01 vs pIEP-lacZ in the absence of BH₄).

### 5. DETAILED DESCRIPTION OF THE INVENTION

Nitric oxide is a biologic mediator derived from the amino acid L-arginine. Nitric oxide synthase (NOS) acts upon L-arginine to oxidize one of the guanidino nitrogens to nitric oxide while citrulline is formed from the remainder of the L-arginine molecule. While it is understood by those skilled in the art that nitric oxide has normal physiologic intracellular and extracellular regulatory functions, excessive production of nitric oxide can be both detrimental and beneficial. It will be appreciated by those skilled in the art that there are no other readily available sources of human tissue inducible nitric oxide synthase.

The present invention relates to gene therapy techniques utilizing a human iNOS DNA sequence to provide therapeutic relief from diseases or disorders such as vascular occlusive disease associated with atherosclerosis, vascular bypass, diabetes mellitus, tumor cell growth associated with cancer, microbial infections, tissue injury and non-healing wounds.

The cloning and expression of a human tissue nitric oxide synthase cDNA of the present invention provides for a source of the enzyme in a sufficiently high concentration for providing a therapeutic purpose.

### 5.1. Isolation and Characterization of a cDNA Clone Coding for a Human Inducble Nitric Oxide Synthase

A process for preparing a cDNA clone coding for a human tissue inducible nitric oxide synthase is provided. This process includes inducing the expression of human tissue nitric oxide synthase in vitro. identifying the presence of human tissue nitric oxide synthase messenger RNA (mRNA) by employing a cross-species cDNA probe capable of hybridizing with the human tissue inducible nitric oxide synthase mRNA, collecting the human tissue poly A mRNA which included the human tissue nitric oxide synthase mRNA, constructing a cDNA library from the human tissue poly A mRNA using a reverse transcriptase enzyme and inserting a strand of the cDNA into a phage vector, screening the cDNA library for human tissue inducible nitric oxide synthase clones with a cross-species iNOS cDNA probe. incubating the phage vector containing the cDNA with a bacteria for forming at least one positive plaque containing the cDNA clone for human tissue inducible nitric oxide synthase, rescuing the cDNA clone from the phage vector by employing a helper phage, and converting the rescued cDNA clone to a plasmid vector for obtaining a full length cDNA clone encoding human tissue inducible nitric oxide synthase.

This process, as hereinbefore described, further includes excising the cDNA insert for human tissue inducible nitric oxide synthase from the plasmid vector. This process also includes confirming the cDNA insert by employing dideoxynucleotide DNA sequencing. Further, this process includes confirming the cDNA insert by employing Southern blot hybridization with a cross-species cDNA probe derived from murine macrophage iNOS.

The process, as hereinbefore described, includes expressing the human tissue inducible nitric oxide synthase cDNA protein in an expression system, such as for example, a bacterial expression system or a mammalian expression system.

It will be appreciated by those skilled in the art that the cloned human inducible nitric oxide synthase cDNA obtained through the methods described herein may be recombinantly expressed by molecular cloning into an expression vector containing a suitable promoter and other appropriate transcription regulatory elements, and transferred into prokaryotic or eukaryotic host cells to produce recombinant inducible nitric oxide synthase. Techniques for such manipulations are fully described in Maniatis, et al., infra, and are well known in the art.

Expression vectors are defined herein as DNA sequences that are required for the transcription of cloned copies of genes and the translation of their mRNAs in an appropriate host. Such vectors can be used to express eukaryotic genes in a variety of hosts such as for example bacteria, bluegreen algae, plant cells, insect cells and animal cells.

Specifically designed vectors allow the shuttling of DNA between hosts such as bacteria-yeast or bacteria-animal cells. An appropriately constructed expression vector should contain: an origin of replication for autonomous replication in host cells, selectable markers, a limited number of useful restriction enzyme sites, a potential for high copy number, and active promoters. A promoter is defined as a DNA sequence that directs RNA polymerase to bind to DNA and initiate mRNA synthesis A strong promoter is one which causes mRNAs to be initiated at high frequency. Expression vectors may include, but are not limited to, cloning vectors, modified cloning vectors, specifically designed plasmids or viruses. A variety of mammalian expression vectors may be used to express recombinant inducible nitric oxide synthase in mammalian cells.

Commercially available bacterial expression vectors which may be suitable for recombinant inducible nitric oxide synthase expression, include but are not limited to, pKC30 (ATCC 37286), pPLa2311 (ATCC 31694), pBR322 (ATCC 31344 and 37017), ptacl2 (ATCC 37138), lambda gt11 (ATCC 37194), pAS1 (ATCC39262), pLC24, pSB226, SV40 and pKK 223-3.

Commercially available mammalian expression vectors which may be suitable for recombinant inducible nitric oxide synthase expression, include but are not limited to, pBC12B1 (ATCC 67617), pMClneo (Stratagene), pXTI (Stratagene), pSG5 (Stratagene), EBO-pSV2-neo (ATCC 37593) pBPV-1(8-2) (ATCC 37110), pdBPV-MMTneo(342-12) (ATCC 37224), pRSVgpt (ATCC 37199), pRSVneo (ATCC 37198), pSV2-dhfr (ATCC 37146), pUCTag (ATCC 37460), and lambda ZD35 (ATCC 37565).

DNA encoding inducible nitric oxide synthase may also be cloned into an expression vector for expression in a recombinant host cell. Recombinant host cells may be prokaryotic or eukaryotic, including but not limited to bacteria, yeast, mammalian cells including but not limited to cell lines of human, bovine, porcine, monkey and rodent origin, and insect cells including but not limited to drosophila derived cell lines. Cell lines derived from mammalian species which may be suitable and which are commercially available, include but are not limited to, CV-1 (ATCC CCL70), COS-1 (ATCC CRL1650), COS-7 (ATCC CRL1651), CHO-K1 (ATCC CCL61), 3T3 (ATCC CCL92), NIH/3T3 (ATCC CRL 1658), HeLa (ATCC CCL2), C1271 (ATCC CRL1616), BS-C-1 (ATCC CCL26) and MRC-5 (ATCC CCL171). The bacterial cell most used for expression of recombinant protein is Escherichia coli. There are various strains of E. coli available and are well known in the art.

The expression vector may be introduced into host cells via any one of a number of techniques including but not limited to transformation, transfection, infection, protoplast fusion, and electroporation.

This process, as hereinbefore described, includes expressing the human tissue inducible nitric oxide synthase protein in a baculovirus expression system.

Another process, as hereinbefore described, includes purifying the human tissue inducible nitric oxide synthase protein.

The process, as hereinbefore described, includes employing as the human tissue inducible nitric oxide synthase a human hepatocyte inducible nitric oxide synthase. This process further includes employing as the human tissue inducible nitric oxide synthase protein a human hepatocyte inducible nitric oxide synthase protein.

A process is provided. as hereinbefore described, including inducing the human tissue nitric oxide synthase in vitro by stimulating a human tissue in vitro with at least one of the following (1) at least one cytokine, such as for example a cytokine selected from the group consisting of tissue necrosis factor (TNF), interleukin-1 (IL-1), and interferon-gamma (IFN-g), (2) at least one bacterial endotoxin including, such as for example, a bacterial lipopolysaccharide (LPS) and (3) combinations thereof.

Another process, as hereinbefore described, includes constructing a cDNA library from the human tissue poly A mRNA which includes the human tissue iNOS mRNA using a reverse transcriptase enzyme and inserting cDNA strands having a length of about at least 1,000 base pairs into the phase vector. Another process is provided, as hereinbefore described, that includes employing lambda Zap II as the phage vector.

Another process is provided, as hereinbefore described, which includes screening the cDNA library by incubating the phage vector for about 6 to 24 hours with a bacteria at a temperature of about 34 to 40 degrees centigrade for effectuating phage lysis of the bacteria. This process further includes rescuing the cDNA clone from the phage vector by employing a helper phage such as for example ExAssist helper phage (Stratagene, La Jolla, Calif.).

Another process, as hereinbefore described, is provided including converting the rescued cDNA clone to the plasmid vector for obtaining a full length cDNA clone encoding the human tissue inducible nitric oxide synthase wherein the plasmid vector includes pBluescript (Stratagene, La Jolla, Calif.).

Another process, as hereinbefore described, includes employing as the human tissue inducible nitric oxide synthase a human hepatocyte inducible nitric oxide synthase.

Another process is provided for producing human hepatocyte inducible nitric oxide synthase protein comprising providing a replicatable DNA expression vector capable of expressing a DNA sequence encoding human hepatocyte inducible nitric oxide synthase in a suitable host, transforming the host to obtain a recombinant host, and maintaining the recombinant host under conditions permitting expression of the DNA sequence to provide human hepatocyte inducible nitric oxide synthase protein.

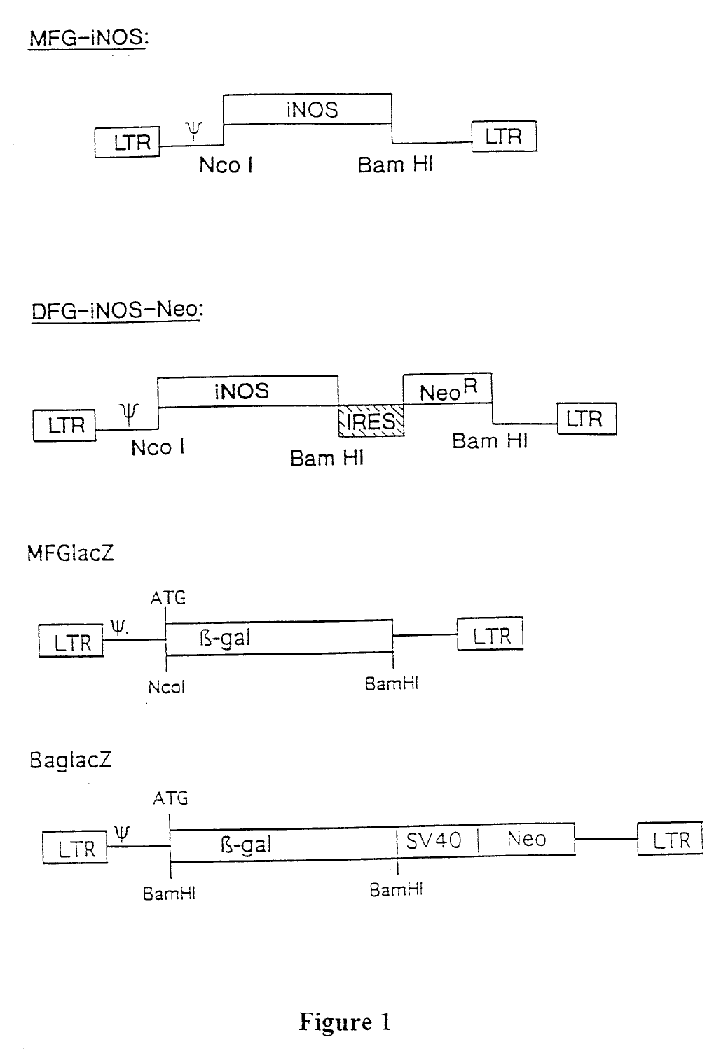

The human hepatocyte inducible nitric oxide synthase cDNA clone has a cDNA coding for the amino acid sequence shown in FIGS. 1A-G. FIGS. 1A-G show the cDNA sense sequence (top line of each horizontal row; SEQ ID NO: 1) and the deduced amino acid sequence of amino acids 1-1153 (bottom line of each horizontal row: SEQ ID NOS: 1 and 2) for the cDNA clone for the human hepatocyte inducible nitric oxide synthase of this invention. FIGS. 1A-G show that the cDNA sequence for the human hepatocyte inducible nitric oxide synthase of this invention is 4,145 nucleotide bases long with the start codon beginning at base number 207 and the stop codon ending at base number 3668. The cDNA double strand sequence was determined using the Sanger dideoxynucleotide sequence technique (Sanger, et al., 1977, Proc. Natl. Acad. Sci. USA 74: 5463-5467) on a Genesis 2000 sequencing system (USB, Cleveland, Ohio).

Another process provides a human tissue inducible nitric oxide synthase recombinant protein expressed from a human tissue inducible nitric oxide synthase cDNA clone. In a preferred embodiment, a human hepatocyte inducible nitric oxide synthase recombinant protein expressed from a human hepatocyte inducible nitric oxide synthase cDNA clone is provided.

A protein comprising a human inducible nitric oxide synthase substantially free of other human proteins is also provided.

An isolated DNA sequence encoding human inducible nitric oxide synthase consisting essentially of an initiation codon positioned upstream and adjacent to an open reading frame consisting essentially of a DNA sequence encoding human inducible nitric oxide synthase is provided.

An isolated DNA sequence encoding human inducible nitric oxide synthase consisting essentially of an initiation codon positioned upstream and adjacent to an open reading frame consisting essentially of a DNA sequence encoding human inducible nitric oxide synthase protein is provided. The human inducible nitric oxide synthase protein begins at the initiation codon and terminates at a stop codon.

A recombinant plasmid is provided containing a recombinant plasmid pHINOS having a deposit accession number ATCC 75358 deposited with the American Type Culture Collection. A further embodiment of this invention provides for bacteria transformed by the recombinant plasmid pHiNOS. A microorganism is provided containing a HiNOS cDNA plasmid transformed in E. coli SOLR bacteria having a deposit accession number ATCC 69126 deposited with the American Type Culture Collection. Both deposits were made under the terms of the Budapest Treaty.

### 5.2. Gene Therapy Applications Utilizing Human iNOS

### 5.2.1. Vascular Gene Therapy Utilizing Human iNOS

Vascular occlusive disease due to atherosclerosis results in significant morbidity in the form of stroke, myocardial infarction, and limb loss. No effective means to resist these changes currently exist. The capacity to bypass occluded vessels is often limited by thrombosis and occlusion of the bypass graft.

Accelerated vascular occlusive disease associated with diabetes mellitus results in a high incidence of myocardial infarction, renal failure, stroke, blindness, and limb loss at an early age. Because smaller sized arteries are often preferentially involved, therapies such as bypass or angioplasty, aimed at alleviating stenotic vessels, are frequently ineffective or complicated by early thrombosis or early restenosis. Factors that contribute to atherosclerosis and diabetic vascular lesions include endothelial injury and dysfunction, macrophage and platelet accumulation, lipid and lipoprotein accumulation, accumulation of glycosylation products, and vascular smooth muscle cell proliferation (Colwell, 1991, Am. J. Med. 90: 6A-50S-6A-54S).

The present invention discloses treatment of vascular diseases or vascular disorders by increasing local iNOS expression through targeting of the mammalian cell populations which form the arterial luminal lining, namely endothelial cells and vascular smooth muscle cells. More specifically, the target mammalian cells may be, but are not necessarily limited to: (1) in vitro cultured endothelial cells and (2) in vitro cultured vascular smooth muscle cells, which may be transfected or infected with a DNA sequence encoding iNOS or a biologically active fragment or derivative thereof and subsequently utilized to repopulate arteries of the patient. It is also within the scope of this invention to use a combination of infected and/or transfected endothelial and vascular smooth muscle cells for repopulation of a diseased vessel.

It will be preferred to utilize endothelial and/or smooth muscle cells obtained from the patient, which will be placed into culture by any number of methods known to one of ordinary skill in-the art. A source of these arterial-based cells may be obtained by harvesting a source these cells from the patient, including but not limited to harvesting a portion of a saphenous vein from the patient. This mode of obtaining target cell source material for in vitro culture prior to iNOS infection or transfection procedures will be especially useful in seeding a synthetic or autologous graft for transfer to the patient.

In another embodiment of the present invention, endothelial cells, vascular smooth muscle cells or a combination of both are targeted for in situ infection or transfection with a DNA sequence encoding iNOS or a biologically active fragment or derivative thereof so as to promote increased local iNOS expression within selected segments of arterial vessels.

Any of the transgenic constructs discussed within this specification may be utilized for either in vitro or in situ applications.

It is a preferred aspect of the invention to utilize a human nucleic acid fragment encoding an iNOS protein or biologically active fragment. In regard to directing a human iNOS construct to the appropriate cell type and arterial location, a further preferred embodiment involves use of the cDNA clone encoding human hepatocyte iNOS or a biologically active fragment thereof. This cDNA clone may be utilized to generate various biologically active iNOS constructs for use in gene therapy applications to increase localized arterial iNOS expression for treatment of vascular diseases including but by no means limited to vascular occlusive disease associated with atherosclerosis and diabetes mellitus, vascular disorders resulting in a high incidence of myocardial infarction, renal failure, stroke. blindness and limb loss at an early age, as well as prevention of intimal hyperplasia. Cell and arterial specific expression of human iNOS in targeted cells will result in local production of prophylactically and therapeutically effective amounts of nitric oxide in the area of expression. Local iNOS expression will promote maximal local vasodilation, resist local thrombosis and potentially retard local vascular smooth muscle cell proliferation, all of which may resist the atherosclerotic disease process and vascular conduit occlusion. However, it will be further understood to one of ordinary skill in the art that any DNA sequence encoding an inducible form of human iNOS, regardless of the tissue source, is a candidate for utilization in gene therapy of vascular occlusive disease in humans. It will be further understood by the skilled artisan that any isolated DNA sequence encoding a protein or protein fragment which mimics the biological activity of human hepatocyte iNOS may be utilized to practice the present invention. Such isolated DNA sequences include, but are not necessarily limited to (1) an isolated cDNA or genomic fragment encoding human hepatocyte iNOS encoding a biologically active fragment thereof; (2) an isolated cDNA, genomic fragment or nucleic acid fragment encoding a biologically active protein or protein fragment of a non-hepatocyte human iNOS; (3) an isolated cDNA, genomic fragment or nucleic acid fragment thereof encoding a biologically active protein or protein fragment thereof of a non-human iNOS; or (4) a synthetic DNA molecule encoding a polypeptide fragment with similar biological activity as described for iNOS.

The DNA sequence encoding iNOS may be delivered to endothelial or vascular smooth muscle target cells by viral or non-viral mediated routes whether the application be in vitro based or in situ based. Virus vectors utilized in the present invention include, but are not limited to (a) retroviral vectors, including but not limited to vectors derived from Moloney murine leukemia virus (MoMLV); (b) adeno-associated vectors; (c) adenovirus vectors; (d) herpes simplex virus vectors; (e) SV40 vectors; (f) polyoma virus vectors; (g) papilloma virus vectors; (h) picornavirus vectors; and (i) vaccinia virus vectors. Depending on the virus vector system chosen, techniques available to the skilled artisan are utilized to infect the target cell of choice with the recombinant viral vector.

By way of example, and not of limitation, a recombinant retroviral vector comprising a DNA sequence encoding iNOS or a biologically active fragment thereof is utilized to infect cultured mammalian endothelial cells which are then used to repopulate arterial vessels or vascular bypass grafts. The retroviral-iNOS recombinant construct is transferred into a standard retroviral packaging cell line and the recovered viral particles are used to infect cultured endothelial cells. These in vitro infected cell populations are then reintroduced into the patient.

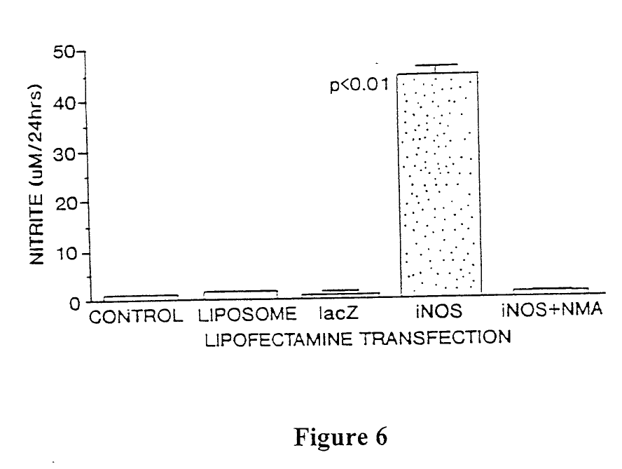

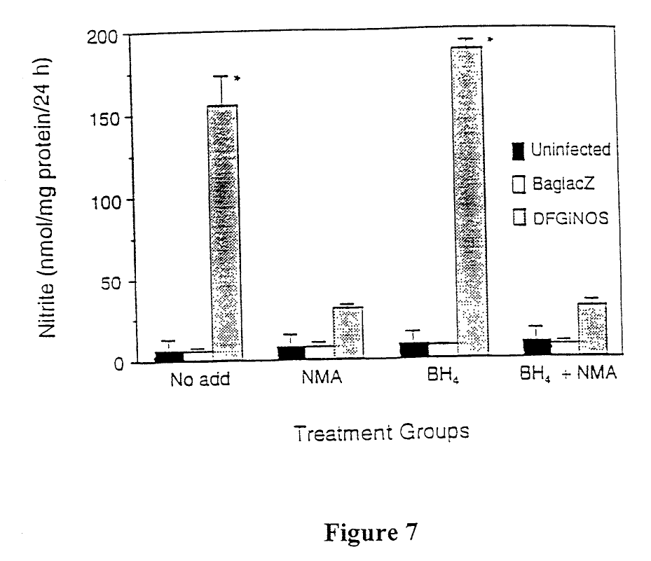

Any number of retroviral constructs which express a biologically active form of iNOS may be utilized by the skilled artisan in practicing the invention. However, a preferred embodiment of the invention depends upon infection of endothelial cells with an iNOS-containing recombinant Moloney murine leukemia virus (MoMLV) retroviral vector. Although MoMLV is a RNA virus, it has a DNA intermediate form that stably integrates into the genome of the host cell. The virus has two long terminal repeats (LTRs) at the 5′ and 3′ end of the proviral DNA that contain promoter, polyadenylation, and integration sequences required for the viral life cycle. A packaging sequence, termed psi, is also required in cis for the production of infectious virus. The virus encodes three proteins, gag, pol, and env, that are required in trans for viral replication. The gag and pot proteins are expressed from a non-spliced message whereas the env protein is expressed from a spliced message generated using the 5′ and 3′ splice sites shown. To generate a recombinant retroviral vector, the gag, pol, and env genes were removed, resulting in the replication deficient MFG derivative of MoMLV. The cDNA encoding iNOS was subcloned into MFG, resulting in MFG-iNOS. In MFG-iNOS, the gene is expressed from a LTR-driven spliced message. The MFG-iNOS construct has the psi site required for packaging of the recombinant RNA into virions. To generate infectious virus, the proviral DNA is transfected into a packaging cell line that constitutively produces gag, pol, and env proteins. FIG. 1A-G shows the sequence of the cDNA encoding the human hepatocyte iNOS and is inserted into the NcoI and BamHI cloning sites of the retroviral vector MFG (FIG. 6 and FIG. 7; for a review of retroviral vectors, see Miller, 1992, Current Topics in Microbiology and Immunology 158: 1-24).

One of ordinary skill in the art will understand any additional isolated DNA sequence or synthetically produced DNA sequence encoding a biologically active portion of iNOS, as hereinbefore disclosed, may be subcloned into a retroviral vector for eventual in vitro or in situ infection of cultured endothelial cells or vascular smooth muscle cells. In vitro infected endothelial cells or vascular smooth muscle cells may then be delivered to specific tissue target sites within the patient as described within this specification. In situ applications will involve direct delivery to the arterial segment to be treated.

The present invention also discloses the use of iNOS-retroviral vectors in gene therapy applications to treat vascular disease by in situ infection or transfection of endothelial cells or vascular smooth muscle cells with a DNA sequence encoding iNOS so as to promote increased local iNOS expression within selected arterial segments or vascular bypass grafts.

In vitro viral-mediated infection or vector-mediated transfection of endothelial cells with a DNA sequence encoding iNOS or a biologically active fragment thereof to treat vascular disease may be accomplished by numerous non-biologic and/or biologic carriers other than the hereinbefore mentioned retroviral vectors.

For example, in an additional embodiment of the invention, a DNA sequence encoding iNOS or a biologically active fragment thereof may be subcloned into an adenovirus viral vector. Any adenovirus (Ad) vector system that will promote expression of iNOS in the target cell of interest may be utilized. Any number of eukaryotic promoters available to one of ordinary skill in the art may be used in constructing an adenovirus-iNOS gene therapy vector. Therefore, any eukaryotic promoter and/or enhancer sequences available to the skilled artisan which are known to control expression of the nucleic acid of interest may be used in Ad vector constructs, including but not limited to a cytomegalovirus (CMV) promoter, a Rous Sarcoma (RSV) promoter, a Murine Leukemia (MLV) promoter, a β-actin promoter, as well as any additional tissue specific or signal specific regulatory sequence that induces expression in the target cell or tissue of interest. Adenovirus gene therapy vectors will be advantageous due to, for example, (1) efficient infection of nondividing cells such as endothelial cells and hepatocytes and, (2) the transient nature of adenovirus vector expression in the target cell, which will be advantageous in applications to prevent thrombosis immediately post-angioplasty.

In addition, a preferred embodiment for in situ applications involve use of an AdiNOS construct. The AdiNOS construct used to exemplify a portion of the invention was constructed as follows. First, the large size of the adenoviral genome requires that it be separated into two separate plasmids before recombinant manipulations can be performed. The plasmid carrying the 5′ portion of the genome was employed for the construction of an adenoviral plasmid carrying the iNOS cDNA. The E1 region of the adenoviral genome was previously deleted from this plasmid and in its place, the full-length iNOS cDNA was inserted along with a CMV enhancer/promoter complex. After this plasmid was generated, it was cotransfected with the plasmid carrying the remainder of the adenoviral genome into 293 cells. These cells constitutively express the E1 gene product and are therefore able to package infectious adenoviral particles from E1 deleted constructs. Following transfection, intracellular recombination occurs to generate the full-length adenoviral genome containing the iNOS cDNA. Infectious AdiNOS particles are then generated and released from the 293 cells through a lytic process and the culture supernatant is collected. This supernatant is subjected to sucrose banding to purify and concentrate the AdiNOS viral particles. The virus can be stored at −80° C. for extended periods of time.

In an additional embodiment of the invention, a DNA sequence encoding iNOS or a biologically active fragment thereof may be subcloned into an adeno-associated viral vector (AAV). One of ordinary skill in the art may construct a recombinant AAV-iNOS vector to be utilized in any one of a number of gene therapy applications. In contrast to retroviral terminal repeat sequences. AAV terminal repeat sequences do not contain regulatory sequences which promote foreign gene expression. As discussed above for Ad vectors, any eukaryotic promoter and/or enhancer sequences available to the skilled artisan which are known to control expression of the nucleic acid of interest may be used in AAV vector constructs, including but not limited to a cytomegalovirus (CMV) promoter, a Rous Sarcoma (RSV) promoter, a Murine Leukemia (MLV) promoter, a β-actin promoter, as well as any additional tissue specific or signal specific regulatory sequence that induces expression in the target cell or tissue of interest.

An appropriate recombinant AAV-iNOS vector can be utilized to directly infect in vitro cultured endothelial cells or vascular smooth muscle cells. Endothelial cells infected with recombinant AAV-iNOS can then be delivered to the specific tissue target site utilizing methods known in the art, including but not limited to the catheterization techniques disclosed within this specification. Alternatively, recombinant AAV-iNOS can be delivered to the target cell through association with liposome microcapsules. A transfection protocol utilizing a hybrid liposome:AAV construct involves using an AAV vector (most likely with both LTR's present) comprising an iNOS DNA sequence. This construct is cotransfected into target endothelial cells or vascular smooth muscle cells with a plasmid containing the rep gene of AAV. Transient expression of the rep protein enhances stable integration of the recombinant AAV-iNOS genome into the endothelial cell or vascular smooth muscle cell genome. To distinguish the transfected iNOS from the small amounts of native iNOS that may be expressed by endothelial cells, the iNOS constructs will include a hemagglutinin epitope tag. The epitope tag will be inserted both 5′ and 3′, and tested for any effects on iNOS activity. An antibody to the hemagglutinin epitope will be used to identify transfected iNOS by methods known to one of ordinary skill in the art. For an example, but not to be construed as a limitation, the CMV promoter-iNOS region of pCIS-iNOS will be ligated between the terminal repeats of AAV. The iNOS-AAV construct will be cotransfected into endothelial cells with a plasmid containing the rep gene of AAV and with lipofectamine (BRL). An assay for iNOS activity will be assayed 48-72 hours later.

In addition to the hereinbefore described use of viral vectors to infect target cells, any known non-viral vector that is capable of expression upon transfection of a specified eukaryotic target cell may be utilized to practice the present invention. Such non-viral based vectors include, but are not solely limited to, plasmid DNA.

One of ordinary skill in the art will be guided by the literature to choose an appropriate DNA plasmid vector for use in the present invention. As discussed above for recombinant Ad and AAV vectors, any eukaryotic promoter and/or enhancer sequences available to the skilled artisan which are known to control expression of the nucleic acid of interest may be used in plasmid vector constructs, including but not limited to a to cytomegalovirus (CMV) promoter, a Rous Sarcoma (RSV) promoter, a Murine Leukemia (MLV) promoter, a herpes simplex virus (HSV) promoter, such as HSV-tk, a β-actin promoter, as well as any additional tissue specific or signal specific regulatory sequence that induces expression in the target cell or tissue of interest. A signal specific promoter fragment includes but is not limited to a promoter fragment responsive to TNF.

In one such embodiment, a DNA sequence encoding human iNOS is subcloned into the DNA plasmid expression vector, pCIS (Genentech), resulting in pCIS-iNOS. pCIS is a standard mammalian expression vector, containing an antibiotic resistance gene for propagation in E. coli and a CMV promoter active in mammalian cells. Such a construct, which may be constructed by one of ordinary skill with components available from numerous sources, will drive expression of an iNOS DNA fragment ligated downstream of the CMV promoter subsequent to transfection of the target cell. More specifically, a NotI/XhoI restriction fragment containing the human iNOS coding region is generated and isolated from pHiNOS (pHiNOS is deposited with the ATCC with accession number 75358) and ligated into NotI/XhoI digested pCIS. Alternatively, the isolated human iNOS sequence may be fused to any portion of the wild type human iNOS promoter sequence such that expression of human iNOS can be induced within the target cell. The pCIS-iNOS utilizes a CMV promoter/enhancer, resulting in high iNOS activity in transient transfection experiments. In addition to the CMV enhancer/promoter sequence of pCIS, sequences downstream of the promoter enhancer fragment of the 5370 bp mammalian expression plasmid include, from 5′-3′, a CMV intron, a polylinker sequence for ligation of the DNA fragment of interest. an SV40 polyadenylation site, an SV40 origin of replication. a DHFR cDNA fragment and the β-lactamase cDNA, which imparts ampicillin resistance. As discussed elsewhere in the specification, any number of mammalian expression vectors may be utilized to deliver the iNOS sequence of interest to the target cell. The pCIS-iNOS DNA will be combined with lipofectamine (BRL) at a ratio of 1 μg DNA/10 nmole liposomes and slowly added to endothelial cells. The cells will be incubated for 5 hours in serum-free media, followed by washing and assay for iNOS activity 48 hours later. The lipofectamine reagent has demonstrated approximately a 10% transfection efficiency in cultured murine endothelial cells. As well as promoting transient and long-term expression of iNOS, liposome transfection of vector DNA comprising an iNOS DNA sequence also provides a system for assay of potential nitric oxide toxicity as discussed above.

In a preferred embodiment utilizing plasmid DNA to transfect target cells, a plasmid vector comprising a DNA sequence encoding iNOS or a biologically active fragment thereof will be utilized in liposome-mediated transfection of the target cell choice as described within this specification. The stability of liposomes, coupled with the impermeable nature of these vesicles, makes them useful vehicles for the delivery of therapeutic DNA sequences (for a review, see Mannino and Gould-Forgerite, 1988, BioTechniques 6(7): 682-690). Liposomes are known to be absorbed by many cell types by fusion. In one embodiment, a cationic liposome containing cationic cholesterol derivatives, such as SF-chol or DC-chol, may be utilized. The DC-chol molecule includes a tertiary amino group, a medium length spacer arm and a carbamoyl linker bond as described by Gao and Huang (1991, Biochem. Biophys. Res. Comm. 179: 280-285). As an example, but not a limitation, the pCIS-iNOS plasmid construction can be utilized in liposome-mediated in vitro transfection of cultured endothelial cells as well as in situ transfection of endothelial cells.

In another embodiment regarding the use of liposome technology, the viral or nonviral based vector comprising the DNA sequence encoding a biologically active iNOS protein fragment is delivered to the target cell by transfection of the target cell with lipofectamine (Bethesda Research Laboratory). Lipofectamine is a 3:1 Liposome formulation of the polycationic lipid 2,3 dioleyloxy-N-[2(sperminecarboxymido)ethryl]-N,N-dimethyl-1-propanaminiumtric fluroacetate (DOPSA) and the neutral lipid dioleoly-phosphatidylethanolamine (DOPE).

Other uses of non-viral modes of gene delivery include, but are not limited to, (a) direct injection of naked DNA; (b) calcium phosphate [Ca₃(PO₄)₂] mediated cell transfection; (c) mammalian host cell transfection by electroporation: (d) DEAE-dextran mediated cell transfection; (e) polybrene mediated delivery; (f) protoplast fusion; (g) microinjection; and (h) polylysine mediated transformation and the genetically engineered cells transferred back to the mammalian host.

The present specification discloses preferred methods of gene therapy-based increase in local human iNOS expression within a targeted region of an artery or within a synthetic conduit utilized to bypass a diseased segment of the arterial vessel.

For example, a preferred method involves in vitro targeting of cultured endothelial cells, vascular smooth muscle cells or a combination of both cell types with a human iNOS DNA fragment ligated into a retroviral vector, such as MFG. A preferred retroviral construct is MFG-iNOS. Such a retroviral vector is transfected into an appropriate packaging cell line to generate infectious virus which is then used to infect endothelial cells, vascular smooth muscle cells or a combination of both cell types in vitro. A direct source of these vascular cells may be obtained, for example, by harvesting a portion of a saphenous vein or any other accessible vein or artery from the patient.

The skilled artisan will have access to numerous endovascular surgical techniques to direct in situ or in vitro based application of prophylactic or therapeutic levels of iNOS to the target cell(s) or region of the arterial vessel. It will be known to the skilled vascular surgeon that various endovascular surgical techniques are available, depending upon the severity of the occlusion and location of arterial vessel target for treatment. For a review of endovascular alternatives, see generally Ahn. 1993, “Endovascular Surgery,” in Vascular Surgery: A Comprehensive Review, Ed. W. S. Moore, W. B. Saunders & Co., Philadelphia). Endovascular surgical procedures include but are not limited to balloon angioplasty, intravascular stents, laser-assisted balloon angioplasty, double balloon catheterization, mechanical endarterectomy and vascular endoscopy. For example, several catheter designs may be utilized for local delivery of an iNOS or iNOS/GTPCH containing entity to the patient. One catheter design consists of two independently inflated balloons; one proximal and one distal to the vascular delivery site. Inflation of these balloons provides an evacuated isolated arterial segment into which vectors for gene delivery can be infused. This system is however limited by a failure to provide distal arterial perfusion. A second catheter design developed by Wolinsky allows the infusion of the iNOS containing carrier through 25-100 μm pores under pressures up to 5 atm. This perfusion pressure increases the depth of penetration by the iNOS vectors and additionally increases gene transfer efficiency. Yet another catheter design utilizes an expandable stent which traps the balloon against the arterial wall and allows intramural delivery of the gene through spaces in the stent material. Additionally, these stents can be modified with burrs which create holes deeper in the vessel wall and allow flow of the gene delivery agents to these sites to allow more uniform delivery of the gene throughout the vessel wall. Another delivery mechanism is to coat the catheter with a hydrophilic polyacrylic acid polymer which acts as a drug absorbing sponge. By disrupting the vessel during the angioplasty procedure, this hydrogel is deposited within the vessel wall and will allow sustained delivery of the vector at the arterial injury site. Additionally, the iontophoretic balloon catheter is a catheter design which uses low electrical current to change the cell membrane polarity and allow the diffusion of charged DNA particles into the cell. This is a potential delivery mechanism for plasmid DNA gene constructs. Also, biodegradable stents formed from agents such an ethylenevinyl acetic copolymer have been utilized to deliver drugs locally at intravascular injury sites and are envisioned for localized delivery to vascular tissue. Alternatively, an intravascular stent may be utilized wherein the endovascular scaffold of the stent is bathed in a ointment, cream, lotion, colloidal dispersion such as a gel or magma or any other acceptable carrier which comprises the iNOS containing entity (or an iNOS-GTPCH containing entity) for delivery to the targeted portion of a vessel segment. This iNOS containing solution (or an iNOS-GTPCH containing solution) may be applicable to either an in situ or in vitro based vessel delivery. Another specific application, offered for the purpose of example and not of limitation, is the use of a self-expanding stent such as a Medinvent stent. This intravascular stent may be bathed in a gel solution comprising an iNOS containing recombinant viral supernatant and delivered percutaneously to the target vessel site. An initial angioplasty, if necessary, is followed by delivery of the bathed scaffold to the target vessel site. The delivery catheter is removed and the scaffold is dilated with a conventional balloon. It will also be within the purview of the skilled vascular surgeon to use other types of intravascular stents such as a balloon expandable stent (e.g., the Palmaz stent) or a thermal expanding stent (e.g., the Cragg stent). Additionally, numerous balloon catheters of varying sizes, shapes, and types of guidelines, some described in this paragraph, are available to the skilled vascular surgeon for endovascular delivery of the iNOS or iNOS-GTPCH composition.

Preferred modes of in vitro infection of arterial luminal cells include human iNOS-containing recombinant retrovirus, especially MFG-iNOS; liposome-mediated transfection of a recombinant iNOS-containing plasmid vector, especially pCIS-iNOS, a recombinant adenovirus vector or a recombinant adeno-associated virus vector. It will be understood to the skilled artisan that similar in vitro infection or transfection procedures may be utilized whether the target cell is an endothelial cell or a vascular smooth muscle cell.

An additional method directing increased local iNOS expression at specific sites within an artery involves iNOS-containing recombinant viral infection of endothelial cells, vascular smooth muscle cells or a combination of both in situ. A stenosis or occluded region of the arterial vessel is substantially cleared such that the cleared region acts as a receptacle for recombinant iNOS viral particles. Endovascular procedures to deliver iNOS-infected endothelial and/or vascular smooth muscle cells are used to repopulate a region of the diseased arterial wall in the same fashion as described elsewhere in this section. Again, these procedures include but are not limited to balloon angioplasty, intravascular stents, laser-assisted balloon angioplasty, double balloon catheterization, mechanical endarterectomy and vascular endoscopy.

Preferred modes of in situ infection of arterial luminal cells include iNOS-containing recombinant viral particles, especially AdiNOS and MFG-iNOS; liposome-mediated transfection of a recombinant iNOS-containing plasmid vector, especially pCIS-iNOS, a recombinant adenovirus vector or a recombinant adeno-associated virus vector. Both endothelial and vascular smooth muscle cells may be infected or transfected simultaneously through in situ procedures, exemplified but not limited to the in situ procedure outlined in the appended Example Sections. Again, endovascular procedures documented in this section may be utilized to infect vascular cells in situ. These procedures include but are not limited to balloon angioplasty, intravascular stents, laser-assisted balloon angioplasty, double balloon catheterization, mechanical endarterectomy and vascular endoscopy.

Additional preferred methods of iNOS based gene therapy treatment of vascular disease involves vascular surgery. More specifically, vascular surgical procedures characterized by:

(1) Infecting or transfecting in vitro cultured mammalian cells selected from the group consisting of endothelial cells, vascular smooth muscle cells or a combination of both cell types with a human iNOS-containing viral or non-viral vector encoding a biologically active iNOS protein or protein fragment; seeding a synthetic or autogenous conduit with a population of the human iNOS-transfected cells; and forming a proximal and a distal anastomosis which bypass a diseased arterial vessel segment within said patient. iNOS-based gene therapy combined with vascular bypass techniques will promote expression of iNOS within the graft, resulting in prophylactic and therapeutic relief by preventing or substantially reducing intimal hyperplasia, thrombogenicity, and other forms of post-operative occlusive complications which commonly occur following vascular bypass procedures.

(2) Infecting or transfecting in vitro cultured endothelial cells, vascular smooth muscle cells or a combination of both with recombinant human iNOS; forming a proximal and a distal anastomosis to bypass a diseased portion of an arterial vessel within said patient; physically segregating each anastomosis subsequent to graft suturing; and seeding the isolated area at and around the distal and the proximal anastomoses with arterial cells infected or transfected with a human iNOS construct to promote increased local iNOS expression within the proximity of the anastomoses.

(3) Forming a proximal and a distal anastomosis to bypass a diseased portion of an arterial vessel within the patient: physically isolating each said anastomosis subsequent to graft suturing; and transfecting cells in situ (endothelial, smooth muscle or both) which line the arterial lumen around the target anastomosis with a human iNOS construct such that localized expression of iNOS imparts prophylactic and therapeutic relief from said human vascular disease and from the development of intimal hyperplasia.

(4) Surgically opening an arterial vessel at a site of lumenal narrowing or occlusion and performing endarterectomy to reestablish patency; seeding the site with cultured endothelial cells or vascular smooth muscle cells carrying a human iNOS construct to increase local iNOS expression to prevent reocclusion; and, closing the surgical site.

(5) Surgically opening an arterial vessel at a site of lumenal narrowing or occlusion and performing endarterectomy to reestablish patency; seeding the surgical site by any of the in situ methods disclosed in this specification or any other in situ technique available to the skilled artisan: and, closing the surgical site.

The preferred means of seeding vascular grafts include endothelial and vascular smooth muscle cells infected or transfected with iNOS-containing recombinant viral particles, preferably a recombinant retroviral particle and especially an MoMLV retroviral particle such as MFG-iNOS; liposome-mediated transfection of a recombinant iNOS-construct, especially pCIS-iNOS, and adenovirus or adeno-associated virus based vector iNOS constructs (either directly as a viral supernatant or via liposome-mediated transfection of the arterial cells).

This specification discloses to the skilled artisan use of any conduit available to the vascular surgeon in classical bypass procedures in the iNOS-based gene therapy procedures described herein. The present invention envisions the use of numerous conduits, including but not limited to venous autografts, (especially the saphenous vein), synthetic grafts (especially polytetrafluoroethylene [PTFE], arterial autografts, umbilical vein autografts, allografts and xenografts.

It is known that BH₄ is an essential component of the NOS enzymatic structure, functioning to maintain the active quaternary configuration of the enzymes. BH₄ biosynthesis begins with GTP which is converted by a sequence of three enzymatic reactions to the active cofactor. An alternative pathway is known to exist that converts the substrate sepiapterin to dihydrobiopterin (BH₂) and then to BH₄. The rate limiting step in the de novo pathway is catalyzed by the enzyme GTP cyclohydrolase I (GTPCH). While most cells express the other enzymes constitutively, GTPCH expression is tightly regulated. In vascular smooth muscle cells, GTPCH is not constitutively expressed but can be induced with cytokines, the signals which also stimulate iNOS expression (Werner, et al., 1990, J. Biol. Chem.: 265: 3189-3192). Thus, the coexpression of GTPCH provides the BH₄ needed to support iNOS activity (Nakayama, et al., 1994, Am. J. Physiol.: 266: L455-460). Therefore, a preferred embodiment of the present invention relates to delivery to the target cell type(s) of a DNA fragment which express iNOS and a DNA fragment expressing GTP cyclohydrolase I (GTPCH). DNA fragments expressing these genes may be delivered as part of the same recombinant vector or on separate recombinant vectors, using any techniques disclosed within this specification or known to the skilled artisan. A preferred embodiment relates to the in situ delivery to a target cell of a DNA fragment expressing GTP cyclohydrolase I in tandem with delivery of a DNA fragment which expresses iNOS so as to promote an enhanced local iNOS-based effect.

The present invention also relates to a method of determining the precise efficacy of a transgenic construction upon in situ infection of a diseased human artery.

One embodiment of determining the precise efficacy of a transgenic construction upon in situ infection of a diseased human artery involves obtaining diseased human arteries by methods known the to skilled vascular surgeon, placing the excised arteries in culture, infecting the cultured arteries with the transgenic construct of interest, and measuring various parameters such as intimal hyperplasia, gene expression, and generation of various metabolites. Exemplified sources for organ culture and testing are human coronary arteries obtained from the extirpated hearts of patients undergoing cardiac transplantation and human peripheral arteries obtained from patients undergoing limb amputation for vascular occlusive disease. Such diseased arteries will be readily available to the skilled vascular surgeon due to the routine performance of amputations. Additionally, fresh cadavers or organ donors may also be a potential source for using diseased human arteries as disclosed in this specification.

A second embodiment in determining the precise efficacy of a transgenic construction upon in situ infection of a diseased human artery involves obtaining porcine artery or artery from another experimental mammalian system either obtained in a diseased state or subjected to injury subsequent to excision from the animal. These arteries are also retrieved by methods known to the to skilled vascular surgeon, followed by placing the excised arteries in culture, infecting the cultured arteries with the gene therapy transgenic construct of interest, and measuring various parameters such a intimal hyperplasia and various metabolite generation, and levels of transgene expression or native gene expression.

A preferred embodiment of determining the precise efficacy of a transgenic construction upon in situ infection of a diseased human artery involves infecting either a diseased human artery or diseased or normal porcine artery with an iNOS-based construct, whereby post infection measurements include. but are not limited to NO₂⁻+NO₃⁻ production. cGMP production and changes in medial thickness of diseased arteries in response to infection of a transgenic iNOS construct. This in vitro based system will also be utilized to assess the level of transgene and endogenous gene expression by RT-PCT analysis. Northern blot analysis, Western blot analysis or enzyme assays.

The in vitro culture and use of a human or another mammalian arterial segment to determine efficacy of transgene constructs include a DNA fragment encoding a full length or biologically active fragment which expresses a protein that supplies cofactors related to iNOS metabolism, including but not limited to GTP cyclohydrolase I as well as genes expressing proteins that interrupt the cell cycle, including but not limited to p21, p53 or Rb.

### 5.2.2. Biologic Therapy by Promotion of Antitumor Effects

The L-arginine:NO pathway has been shown to be involved in antitumor activity (Hibbs, et al., 1987, Science 235: 473476; Kilbourn, et al., 1990, Proc. Natl. Acad. Sci. USA 87: 3629-3632). The biological activity of nitric oxide is thought to include inhibition of DNA synthesis and mitochondrial enzymes involved in respiration.

The present invention discloses methods of human iNOS-directed gene therapy to promote antitumor effects in cancer patients. Such a human iNOS-directed cancer gene therapy will provide a local increase in nitric oxide concentration within the area of the tumor to be treated, thus promoting antitumor activity without systemic increases in nitric oxide levels.

Therefore, the present invention discloses targeting of a DNA sequence to specific sites within a patient such that local expression of iNOS will lead to increased nitric oxide concentration, thus stimulating antitumor activity.

The isolated human iNOS DNA sequence may be manipulated in vitro in a number of ways available to one of ordinary skill in the art so as to promote local expression of recombinant iNOS or a biologically active fragment thereof.

The human iNOS DNA sequence encoding the intact iNOS protein or a partial DNA sequence thereof encoding a biologically active fragment thereof will be delivered to the target cell by in vitro transduction utilizing the viral and non-viral methods discussed in Section 5.2.1. The in vitro transduced target cell is then introduced into the patient so as to promote local iNOS expression at the tumor site. Therefore, it will be understood that any human iNOS DNA sequence, regardless of tissue source, is a candidate for cancer gene therapy. Such an iNOS DNA sequence may include, but is not limited to, (1) an isolated cDNA or genomic sequence purified from human hepatocyte cells, or a DNA sequence from said source which encodes a biologically active fragment of human iNOS; or (2) an isolated cDNA or genomic fragment purified from a human non-hepatocyte source, or a DNA sequence from said source which encodes a biologically active fragment of human iNOS.

Any of the above-identified iNOS sequences may be fused to a tissue specific or signal specific promoter fragment active within the target cell, or alternatively, may be fused to the wild type human iNOS promoter sequence. An example of a signal specific promoter in iNOS-driven biologic therapy applications would include, but is not limited to, a promoter upregulated in response to TNF. Therefore, any promoter or enhancer sequence which increases the local expression of iNOS within the transformed target cell is a candidate for use in antitumor applications.

Promotion of local expression of iNOS at or around the tumor site is dependent on utilizing an appropriate target cell for in vitro transduction and introduction into the patient. In one embodiment regarding cancer gene therapy, the patient is intravenously injected with in vitro transduced target cells, including but not limited to tumor infiltrating lymphocytes originally harvested from the patient.

The delivery to the target cell may be accomplished by viral or non-viral methods primarily as described in Section 5.2.1. These methods include, but are not limited to (a) retroviral vectors, including but not limited to vectors derived from Moloney murine leukemia virus (MoMLV); (b) adeno-associated vectors; (c) adenovirus vectors; (d) herpes simplex virus vectors; (e) SV40 vectors; (f) polyoma virus vectors; (g) papilloma virus vectors; (h) picornavirus vectors; and (i) vaccinia virus vectors. Depending on the vector system chosen, techniques available to the skilled artisan are utilized to infect the target cell of choice with the recombinant virus vector.

In a preferred method of delivering a human iNOS sequence to the target cell of interest, a recombinant retroviral vector carrying a DNA sequence encoding iNOS or a biologically active fragment thereof is utilized to infect tumor infiltrating lymphocytes. These infected tumor infiltrating lymphocytes are then reintroduced into the patient to promote local production of nitric oxide at the tumor site.

Any number of retroviral constructs which express a biologically active form of iNOS may be utilized to promote antitumor activity. Preferably, MFG-iNOS or DFG-iNOS-Neo may be utilized to infect cultured tumor infiltrating lymphocytes or tumor cells harvested from the patient.

One of ordinary skill in the art will understand that any additional isolated DNA sequence encoding a biologically active portion of iNOS, as hereinbefore disclosed, may be subcloned into a retroviral vector for eventual in vitro infection of cultured tumor infiltrating lymphocytes or tumor cells harvested from the patient.

In addition to the hereinbefore described use of viral vectors to infect target cells, any known non-viral vector that is capable of expression upon transfection of a specified eukaryotic target cell may be utilized to practice the present invention. Such non-viral vectors include, but are not solely limited to, plasmid DNA.

One of ordinary skill in the art will be guided by the literature to choose an appropriate plasmid vector for use in the present invention. Any eukaryotic promoter and/or enhancer sequence available to the skilled artisan which is known to control expression of the nucleic acid of interest may be used in plasmid vector constructs, including but not limited to a cytomegalovirus (CMV) promoter, a Rous Sarcoma (RSV) promoter, a Murine Leukemia (MLV) promoter, a β-actin promoter, as well as any additional tissue specific or signal specific regulatory sequence that induces expression in the target cell or tissue of interest. In a specific embodiment of the invention, the plasmid vector comprising an iNOS DNA sequence is pCIS-iNOS.

Delivery of iNOS-plasmid constructs to a target cell type, such as tumor cells, may be accomplished by numerous biologic and non-biologic carriers available to one of ordinary skill in the art. In a preferred embodiment utilizing plasmid DNA to transfect target cells, a plasmid vector comprising a DNA sequence encoding iNOS or a biologically active fragment thereof will be utilized in liposome-mediated transfection, as described in detail in Section 5.2.1.

Other uses of non-viral modes of gene delivery include, but are not limited to, (a) direct injection of naked DNA; (b) calcium phosphate [Ca₃(PO₄)₂] mediated cell transfection; (c) mammalian host cell transfection by electroporation: (d) DEAE-dextran mediated cell transfection; (e) polybrene mediated delivery; (f) protoplast fusion; (g) microinjection; and (h) polylysine mediated transformation; and the genetically transformed cells then transferred back to the mammalian host.

iNOS-directed cancer therapy may be exemplified by harvesting and selective culture of a patients tumor infiltrating lymphocyte population, transduction by an iNOS containing viral or non-viral vector, followed by reintroduction of the iNOS-transduced cell to the patient. Peripheral blood lymphocytes are removed from the patient and TILs are selected in culture as described in Rosenberg, et al. (1992, Human Gene Therapy 3:57-73, herein incorporated by reference). The TILs will then be utilized as the target cell population for transduction with DFG-iNOS-Neo^{R}. Transduced TILs are selected in G418-supplemented medium and prepared for administration back to the patient by known techniques.

Another method of iNOS-directed therapy for treating cancer is direct delivery of MFG-iNOS to tumor site(s) in liposome capsules. The stability of liposomes, coupled with the impermeable nature of these vesicles makes them useful vehicles for the delivery iNOS containing sequences, such as but not limited to pCIS-iNOS, to the tumor site. Site specific delivery of the liposome capsule to the tumor site is promoted by modification of the liposome membrane to exhibit a tumor specific antibody so as to promote liposome adhesion and fusion only to the tumor cell. Local delivery of a recombinant iNOS vector to the tumor site will result in increased local iNOS expression, increased nitric oxide production and hence, antitumor activity.

pCIS-iNOS liposomes will be formulated into a suitable pharmaceutical carrier for in vivo administration by injection or surgical implant.

The present invention also relates to in situ iNOS-based treatment of hepatocellular carcinomas, including malignant epithelial neoplasms of the liver, as well as liver metastases. A preferred method of treating liver cancer in situ involves an intravenous, systemic administration of an AdiNOS construct, which will result in an approximately 95% targeting to the liver. These treatments will be available for use alone or in tandem with one or more of recognized systemic or intrahepatic arterial chemotherapy regimes, cytokine immunotherapy (especially including TNF-α) procedures, and radiation therapy, all useful in treating various stages of hepatic tumors. Another embodiment of the present invention relates optimizing the antitumor effect generated by local iNOS expression by the concomitant in situ delivery of a DNA fragment expressing GTP cyclohydrolase I.

A preferred embodiment of a tandem delivery DNA fragments expressing iNOS and GTP cyclohydrolase I provides for use of a recombinant adenovirus viral vector or vectors to direct delivery to the liver to maximize in situ treatment of hepatocellular carcinomas.

### 5.2.3. Biologic Therapy for Treating Microbial Infections

The human iNOS DNA sequences of the present invention may be utilized in treating microbial infections. Specifically, iNOS-driven biologic therapy will be utilized to treat microbes known to be susceptible to increased concentrations of nitric oxide. Nitric oxide is known to be a cytotoxic effector molecule against mycobacteria, helminths, fungi protozoa, and viruses.

Upon review of this specification, the artisan of ordinary skill will be directed to utilize any of the iNOS sequences listed in Section 5.2.1. iNOS-driven antimicrobial therapy is dependant on targeting the respective human iNOS sequence to the tissue-specific cell type harboring the microbe or to the microbe itself. Depending upon the targeted microbe or cell type, delivery of the human iNOS DNA may be accomplished by biologic or non-biologic means.

In a preferred embodiment of utilizing iNOS-driven antimicrobial therapy, the target cells are human hepatocytes infected with the sporozoa Plasmodium, the causative agent of malaria. Human malaria is caused by one of four species of Plasmodium: P. falciparum, P. malanae, P. vivax and P. ovale. The sporocytes of Plasmodium penetrate hepatocyte cells subsequent to entry into the circulatory system of the human host.

A preferred embodiment for utilizing iNOS-driven antimicrobial therapy is targeting human hepatocytes with AdiNOS. The liver is directly targeted by systemically delivering a recombinant adenovirus which expresses iNOS. Therefore, a preferred method of in situ treatment of a microbial infection involving the liver involves an intravenous, systemic administration of an AdiNOS construct, which will result in an approximately 95% targeting of a recombinant AdiNOS vector to the liver. Targeted delivery and expression of iNOS within the diseased tissue will result in high local concentration of iNOS and concomitant therapeutic effects within the diseased tissue.

One preferred embodiment of treating malaria via iNOS-driven biologic therapy, the iNOS-vector is delivered via liposome mediated transformation of the target hepatocytes. An additionally preferred method of treating malaria in the present invention involves targeting human hepatocytes with AdiNOS. Again, the liver is directly targeted by systemically delivering a recombinant adenovirus which expresses iNOS. Therefore, a preferred method of in situ treatment of malaria will also include an intravenous, systemic administration of an AdiNOS construct, which will result in an approximately 95% targeting of a recombinant AdiNOS vector to the diseased tissue.

In another embodiment of the invention, iNOS driven antimicrobial therapy is utilized to treat helminthic infections, including but not solely limited to Schistosomiasis (e.g., Schistosoma mansomi, Schistosoma haematobium, and Schistosoma japonicum. Direct treatment of helminth infected liver cells encompass all techniques described above for iNOS driven therapy of malaria.

In an especially preferred embodiment of treating malaria via iNOS-driven antimicrobial therapy, the liposomes are modified by insertion of an hepatocyte specific asialoprotein into the liposome complex. The resulting asialoprotein binds to the galactose receptor unique to hepatocytes (see Wall, et. al., 1980, Cell 21: 79-83). Therefore, encapsulating the iNOS DNA vector within an asialoprotein-containing liposome will direct delivery specifically to hepatocytes.

Another embodiment of utilizing iNOS-vectors in antimicrobial therapy involves treatment of lung borne microbial infections, including but not limited to tuberculosis and leprosy.

The causative agent of tuberculosis is Mycobacterium tuberculosis, which enters the lung via droplet nuclei and the respiratory route. Once in the lungs, this bacterium grows and eventually is surrounded by lymphocytes, macrophases and connective tissue, forming nodules called tubercles. Normally, this represents the end stage of the infection, with no ill effects. Alternatively, a caseous lesion may form, which may calcify to form a Ghon complex and further become liquified, forming tuberculous cavities.

A preferred treatment of tuberculosis by iNOS-driven antimicrobial therapy involves directing an iNOS vector to the target tissue by viral mediated transformation of cells within the target tissue.

One preferred method of viral mediated delivery is retroviral mediated delivery, as discussed in Section 5.2.1. With the aid of this specification, it is within the realm of the artisan of ordinary skill to construct an iNOS vector for use in treating tuberculosis.

Another preferred method of viral mediated delivery is adenovirus mediated delivery, wherein the iNOS DNA fragment of interest is inserted into an adenovirus vector.

A preferred method of administering iNOS-retroviral or iNOS-adenoviral vectors to infected regions of lung is inhalational administration, in the form of an aerosol mist.

The target would be advanced disseminated disease including but not limited to the treatment of tuberculosis, as well as other microbial infections such as fungal infections in a transplant patient, and disseminated aspergillosis or fungal or additional viral infections such as cytomegalovirus in an AIDS patient.

The causative agent of leprosy is Mycobacterium leprae. Transmission of leprosy is highest when children are exposed to infected individuals shedding M. leprae. Nasal secretions are the most likely infectious material within family contacts. The preferred mode of iNOS viral delivery is through inhalational administration, as described for M. tuberculosis, is also the preferred mode of treating M. leprae.

The treatment of microbial infections by increasing local iNOS expression will be exemplified through the treatment of a malarial infection. The recombinant plasmid vector pCIS-NOS will be delivered locally to the liver in liposome capsules. The liposome capsules will be modified to exhibit a liver specific surface ligand. An asialoprotein is a glycoprotein treated to remove sialic acid (i.e., neuraminic acid). The resulting asialoprotein specifically binds to the galactose receptor unique to hepatocytes (see Wall, et. al., 1980, Cell 21: 79-83). Therefore, encapsulating pCIS-iNOS within an asialoprotein-containing liposome will ensure delivery to and local expression of iNOS in hepatocytes only.

The pCIS-iNOS vector incorporated into liposomes will be formulated into a suitable pharmaceutical carrier for in vivo administration by any appropriate route including but not limited to injection, absorption through epithelial or mucocutaneous lining or by a sustained released implant, whether it be a cellular or tissue implant.

Another embodiment of the present invention relates optimizing the antimicrobial effect generated by local iNOS expression by the concomitant in situ delivery of a DNA fragment expressing GTP cyclohydrolase I.

A preferred embodiment of a tandem delivery DNA fragments expressing iNOS and GTP cyclohydrolase I provides for use of a recombinant adenovirus viral vector or vectors to direct delivery to the liver to maximize in situ antimicrobial treatment.

As related to targeting the liver to treat liver cancer and microbial infections of the liver, the present invention also relates to treatment of various liver injuries. Hepatotoxins which may provoke injury to the liver which are amenable to iNOS gene therapy include but are not limited to acetaminophen, isoniazid, α-methyldopa, chlorpromazine, methotrexate, halothane and tetracycline. Applications of an iNOS expressing transgene construct will also be useful in overcoming TNF-α toxicity sometimes associated with liver injury as seen in inflammation associated with hepatitis. Therefore, a preferred method of in situ treatment of liver injuries which involves an intravenous, systemic administration of an AdiNOS construct, which will result in an approximately 95% targeting of a recombinant AdiNOS vector to the liver and in turn an optimal therapeutic effect.

Another embodiment of treating liver injuries will also entail optimizing the iNOS based effect by means of the concomitant in situ delivery of a DNA fragment expressing GTP cyclohydrolase I. A preferred embodiment of a tandem delivery DNA fragments expressing iNOS and GTP cyclohydrolase I provides for use of a recombinant adenovirus viral vector or vectors to direct delivery to the liver to maximize in situ treatment of these various liver injuries.

### 5.2.4. Biologic Therapy for Treating Non-healing Wounds

The present invention also relates to gene therapy applications to promote wound healing. Nitric oxide has been shown to promote angiogenesis in mice deleted for the iNOS gene. When these mice are subjected to wounding they shows a propensity for faster healing when administered an iNOS source compared to a control wherein a source of iNOS is not supplied. Therefore, a preferred embodiment of the present invention to promote wound healing relates to direct application of iNOS to the wound. Any pharmaceutically effective composition comprises an iNOS source may be applied directly to the wound. A preferred method of treating non-healing wounds with iNOS is to promote optimal infection of the wound area with a recombinant iNOS vector incorporated into a pharmaceutically effective carrier. A further preference is the application of AdiNOS to the non-healing wound, with an especially preferred method involving application of an AdiNOS composition to a non-healing leg ulcer to promote on site angiogenesis.

As noted with other iNOS based gene therapy applications, a concomitant in situ delivery of a DNA fragment expressing GTP cyclohydrolase I along with a DNA fragment encoding iNOS will be an additional embodiment of the present invention.

A preferred embodiment of a tandem delivery DNA fragments expressing iNOS and GTP cyclohydrolase I utilizes a recombinant adenovirus viral vector or vectors for tandem in situ delivery of DNA fragments expressing iNOS and GTP cyclohydrolase I.

### 6. Example

### Isolation and Characterization of Human iNOS

U.S. Pat. No. 5,468.630, issued to Billiar et al. on Nov. 21, 1995, discloses the human iNOS cDNA sequence. The plasmid pHiNOS comprises the human iNOS coding region and was deposited under the terms of the Budapest Treaty on Nov. 20, 1992 an has the ATCC accession number 75358 (pHiNOS) and ATCC accession number 69126 (pHiNOS transformed in E. coli SOLR).

The pHiNOS cDNA was isolated and characterized as disclosed in U.S. Pat. No. 5,468,630, issued to Billiar et al. on Nov. 21, 1995. The nucleotide sequence (SEQ ID NO:1) and amino acid sequence (SEQ ID NO:2) are shown in FIG. 1A-G.

iNOS RNA is weakly induced in hepatocytes following stimulation with individual cytokines such as for example tumor necrosis factor (TNF), interleukin-1 (IL-1) or interleukin-gamma (IFN-g). Cytokines can synergize to further up-regulate iNOS mRNA levels and nitric oxide synthase activity. Maximum induction of iNOS was achieved with a combination of TNF, IL-1, IFN-g and bacterial lipopolysaccharide (LPS). (Geller, et al., 1993, Proc. Natl. Acad. Sci. 90: 522-526; Nussler, et al., 1992, J. Exp. Med. 176:261-264).

A cross-species iNOS cDNA probe capable of hybridizing with human hepatocyte inducible nitric oxide synthase mRNA was used to identify and isolate the mRNA for human hepatocyte inducible nitric oxide synthase. The time-point of peak iNOS mRNA levels following cytokine and LPS [hereinafter cytokine mixture (CM)] stimulation was then determined.

Total cellular RNA was extracted 2-48 hours following CM-stimulation of cultured human hepatocytes using the RNAzol B modified method of Chomczynski and Sacchi (1987, Anal. Biochem. 162:156-159). 20 microgram aliquots of total RNA were examined by Northern blot analysis through cross-species hybridization with a murine macrophage iNOS cDNA probe generated from a fragment of the murine iNOS cDNA isolated after NotI restriction enzyme digest. (Lowenstein, et al., 1992, Proc. Natl. Acad. Sci. USA. 89:6711-6715; GenBank Accession No. M92649). The presence of human hepatocyte nitric oxide synthase mRNA was identified as a single band of about 4.5 Kb size with maximal iNOS mRNA levels seen about 8 hours after CM stimulation.

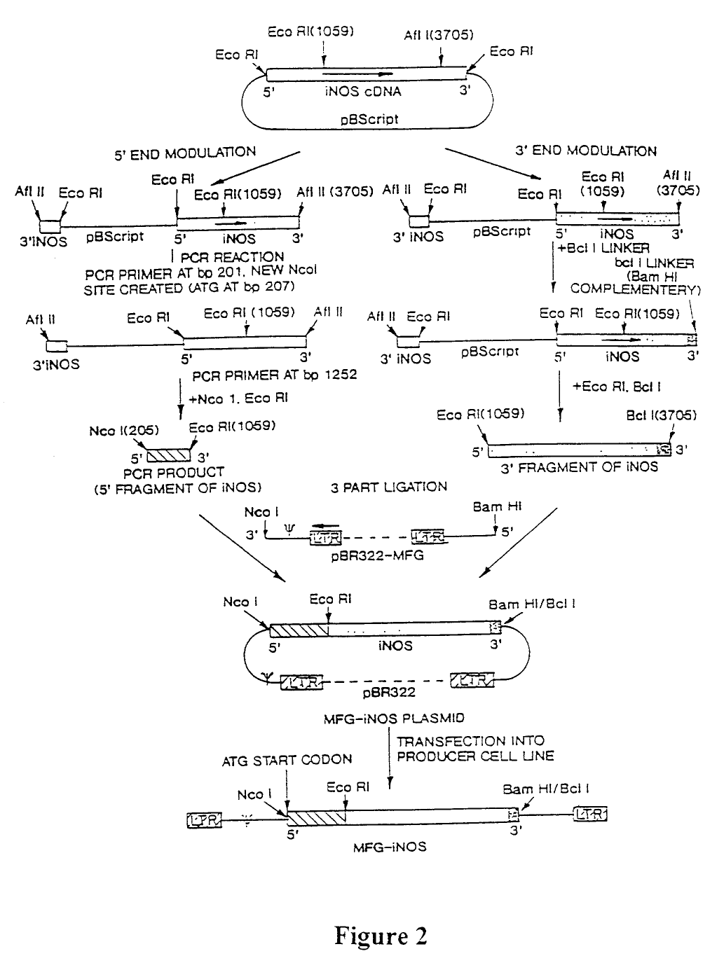

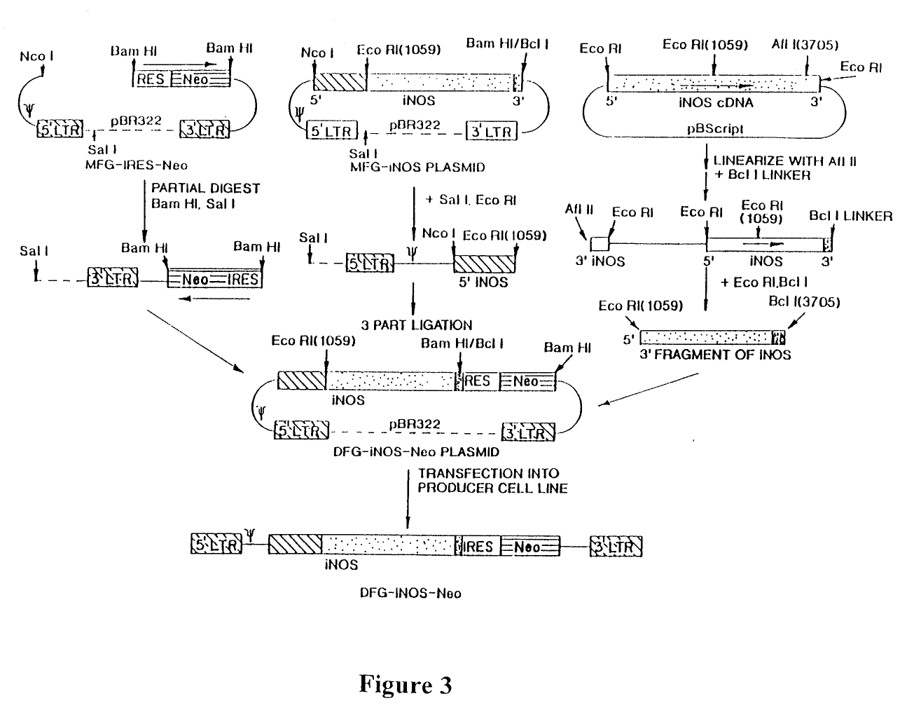

FIG. 2 shows the presence of the 4.5 kb message for human hepatocyte inducible nitric oxide synthase. Freshly isolated human hepatocytes (HC) were placed in cell culture and exposed to a combination of human recombinant tumor necrosis factor (500 units/milliliter), human recombinant interleukin-1 (5 units/milliliter), human recombinant interferon-gamma (100 units/milliliter), and lipopolysaccharide (10 micrograms/milliliter). FIG. 2 shows that total RNA was isolated at the indicated time points (2, 4, 6, & 8 hrs.) and 20 micrograms per sample was subjected to Northern blot analysis. A 2.7 Kb cDNA fragments for murine macrophage inducible nitric oxide synthase was used to identify the human hepatocyte inducible nitric oxide synthase mRNA. FIG. 2 demonstrates that the 4.5 Kb message level peaked at about 8 hours following stimulation. FIG. 2 shows that no mRNA signal was detected in control (unstimulated) hepatocytes. FIG. 3 shows the expression of the 4.5 Kb human hepatocyte inducible nitric oxide synthase mRNA at about 8 hours after exposure to the above mentioned cytokines from hepatocytes isolated from three separate individuals [patent (Pt.) 1, 2, and 3]. FIG. 3 demonstrates that no signal was detected in control (unstimulated) hepatocytes.

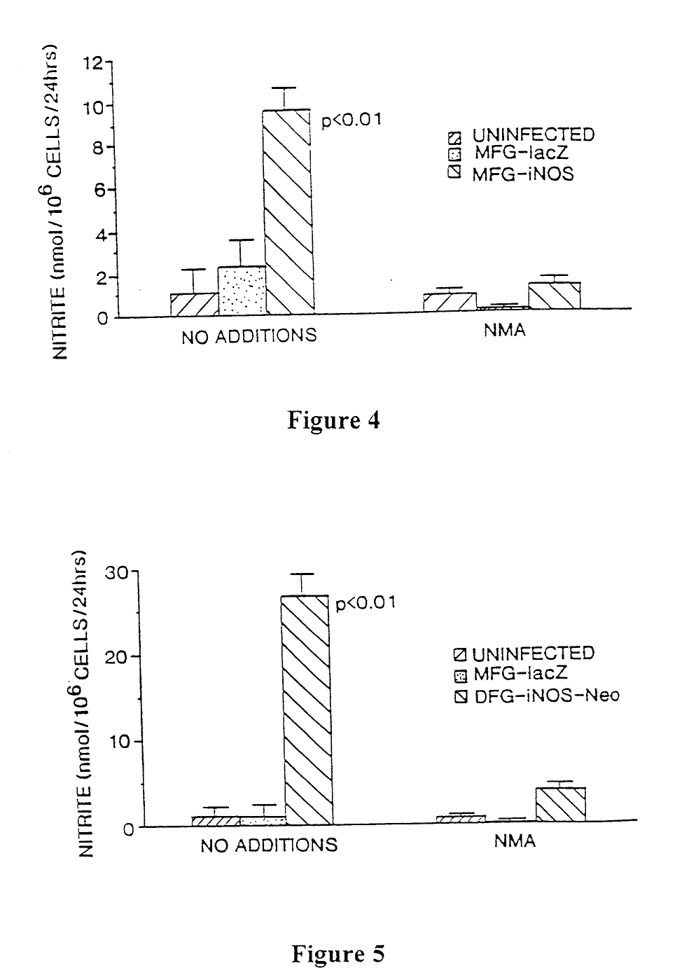

Because the 8 hour time point yielded maximal iNOS mRNA levels, total cellular RNA was isolated from two human livers about 8 hours following CM-stimulation in vitro. cDNA synthesis requires about 10 to 20 micrograms of poly A mRNA rather than total RNA. Poly A mRNA was purified from total cellular RNA by elution through an oligo-dT cellulose column. To identify the presence of human hepatocyte iNOS mRNA in the purified poly A mRNA, repeat Northern blot analysis was performed on 0.5 micrograms of purified A mRNA from each of the two human livers using the 2.7 Kb cDNA probe for murine macrophage inducible nitric oxide synthase. FIG. 4 shows strong nitric oxide synthase mRNA bands from the 2 different patients without evidence of degraded poly A RNA.

FIG. 4 shows that the murine macrophage inducible nitric oxide synthase cDNA probe effectively cross hybridizes and identifies the human hepatocyte inducible nitric oxide synthase mRNA in the poly A RNA. The samples of poly A mRNA from the 2 patients were pooled and were used to construct the cDNA library for isolation of cDNA clone for the human hepatocyte inducible nitric oxide synthase.

Using about 20 micrograms of the poly A mRNA isolated from CM-stimulated human hepatocytes, a cDNA library was constructed by Stratagene, La Jolla, Calif. The first strand cDNA was synthesized from the human hepatocyte poly A mRNA using MoMLV reverse transcriptase enzyme with oligo-dT primers. After excluding strands less that 1000 nucleotide basis pairs in length the cDNA's were inserted into a lambda Zap II phage vector (Stratagene, La Jolla. Calif.) and was titered.

To screen the cDNA library, 1×10⁶ phage were incubated with bacteria (E. Coli Sure strain) at 34 to 40 degrees centigrade for 15 to 30 minutes. This mixture was added to molten agarose and poured onto 20×20 centimeter agar plates at a density of 2×10⁵ plaques/plate (Maniatis et al., 1982, Molecular Cloning: A Laboratory Manual, Cold Spring Harbor Laboratory, Cold Spring Harbor, N.Y.). The plates were incubated at 34 to 40 degrees centigrade overnight for 6 to 24 hours to allow for phage lysis of bacteria. The plaques were then transferred to nitrocellulose filters and clones carrying iNOS cDNA inserts were identified by filter hybridization with ³²P-labeled murine macrophage inducible nitric oxide synthase cDNA probe. Positively labeled clones were cored from the agar plates after localization by autoradiograph alignment. The positive clones were rescued from the lambda Zap II phage vector with the helper phage ExAssist (Stratagene, La Jolla, Calif.), and then converted to plasmid vectors using pBluescript (Stratagene, La Jolla, Calif.). The cDNAs for human hepatocyte inducible nitric oxide synthase were excised from the Bluescript plasmid cloning sites by restriction with EcoRI enzyme and then sized by gel electrophoresis to identify a full-length clone. The cDNA identities were confirmed by DNA sequencing and by Southern blot hybridization with the murine macrophage iNOS cDNA probe. In addition, Northeern blot analysis of cytokine-stimulated human hepatocyte poly A mRNA was performed using the full-length human inducible nitric oxide synthase cDNA clone of this invention as the probe. FIG. 5 shows a time course for the expression of human hepatocyte inducible nitric oxide synthase mRNA. This RNA was isolated from a patient different from the patients listed in FIGS. 2 and 3. The cells of the patient in FIG. 5 were exposed to the same agents as described for FIG. 2. FIG. 5 shows the cloned human inducible nitric oxide synthase cDNA identifies the same size mRNA signal as the murine macrophage iNOS cDNA probe, thus, further confirming its identify. It is important to note that the isolated cDNA clone coding for human inducible nitric oxide synthase of this invention can hybridize with human inducible nitric oxide synthase mRNA, thus, confirming the capacity of the cDNA clone of this invention to identify the human hepatocyte inducible nitric oxide synthase mRNA.

The plasmid vector pBluescript contains universal primer regions which were used to facilitate double-stranded DNA sequencing. Positive clones were sequenced by using the dideoxynucleotide technique of Sanger, supra, with the Genesis 2000 sequencing system (USB, Cleveland, Ohio). Sequence analysis was done using Genbank DNA sequencing software programs available through the Pittsburgh Supercomputing Center (Billiar TR., Pittsburgh Supercomputing Center, Pittsburgh, Pa.).

Verification of the full length cDNA identity was accomplished by expressing the recombinant human hepatocyte inducible nitric oxide synthase protein. The human hepatocyte inducible nitric oxide synthase cDNA was ligated into the pCIS expression vector (Genentech, CA) which utilizes a CMV promoter. Next the expression vector was transfected into human embryonic kidney 293 cells (ATCC, Maryland). Nitric oxide synthase activity was assessed by measuring the conversion of [³H] arginine to [³H] citrulline. It will be appreciated by those skilled in the art that this expression system was successfully used for expression of the cloned rat brain constitutive nitric oxide synthase, and there was negligible nitric oxide synthase activity in the unstimulated 293 kidney cells (Bredt et al., 1991, Nature, 351:714-718). After the identity of the human hepatocyte inducible nitric oxide synthase cDNA clone of this invention was verified as hereinbefore described, the cDNA was expressed in a baculovirus expression system (Invitrogen, San Diego, Calif.) which allowed for large scale enzyme production (1988, Texas Agriculture Experiment Station Bulletin, No. 1555). More specifically, the human hepatocyte nitric oxide synthase cDNA was inserted into the baculovirus transfer vector and then co-transfected with wild type viral DNA into Sf9 insect cells (ATCC, Maryland). Recombinant viral plaques were isolated to allow for protein over-expression.

The resultant human hepatocyte inducible nitric oxide synthase protein was purified using a two step procedure. First, the protein was passed through an anion-exchange column of DEAE cellulose. This was followed by affinity chromatography with 2′, 5′-ADP Sepharose. (Evans et al., 1992, Proc. Natl. Acad. Sci. USA, 89:5361-5365). Purity was assessed by SDS-polyacrylamide gel electrophoresis. Activity was quantitated after each step by measuring the ability of the enzyme to generate NO₂− and NO₃− from L-arginine. NO₂− and NO₃− was measured using an automated colorimetric reaction based on the Green reaction (Green, et al., 1982, Anal. Biochem. 126:131-137).

### 7. Example

### Treatment of Vascular Occlusive Disease

### Retrovirus and Adenovirus Recombinant Constructions

### 7.1. Example

### General Materials and Methods

Total RNA extraction, Northern blots, Southern blots, Western blots, and PCR are techniques routinely performed by one of ordinary skill in the art (e.g., see generally Maniatis, et al., 1989, Molecular Cloning: A Laboratory Manual. Cold Spring Harbor Laboratory, Cold Spring Harbor, N.Y.; Geller, et al., Proc. Natl. Acad. Sci. USA 90: 522-526; Towbin, et al., 1979, Proc. Natl. Acad. Sci. USA 76: 4350; Brenner, et al., 1989, BioTechniques 7: 1096-1103). Additional detail is provided throughout Example Section 13.

### 7.1.1. Recombinant Viral Constructs

a) MFG-iNOS (FIG. 6)

A first exemplified retroviral vector is constructed using as starting materials the human hepatocyte iNOS cDNA construct and MFG, a simplified MoMVL vector in which the DNA sequences encoding the pol and env proteins have been deleted so as to render it replication defective. The majority of the gag sequence has also been deleted. The human hepatocyte iNOS cDNA was inserted into the NcoI and BamHI cloning sites of the retroviral vector MFG as shown in FIG. 6 and FIG. 7. Briefly, the MFG vector has a unique cloning region consisting of a 5′ NcoI site and a 3′ BamHI site. PCR primers were used to generate a point mutation at bp 205 of the iNOS cDNA, manufacturing an NcoI site that incorporated the ATG start codon. A 5′ fragment of the PCR product of the iNOS cDNA spanning from the NcoI site at bp 205 to the EcoRI site at bp 1059 was isolated. The 3′ BamHI site was generated by linearizing the pBScript-iNOS plasmid with AflII which uniquely cut at bp 3705 of the iNOS cDNA. This restriction site is located approximately 40 bp downstream from the iNOS stop codon. A BclI linker was then ligated to the linearized plasmid. Double digestion with EcoRI and BclI allowed the isolation of a 3′ fragment of the iNOS cDNA from bp 1060 (EcoRI) to bp 3710 (BclI). The BclI overhang is complementary to the overhang generated by BamHI. A three part ligation was then performed between MFG, the 5′ PCR product with the 5′ NcoI site, and the 3′ fragment with the 3′ BclI linker. Escherichia coli were transformed with the ligation mixture and grown on ampicillin selection. Transformants were isolated and screened for the properly reconstituted MFG-iNOS construct. One correct transformant was isolated and a large scale plasmid DNA preparation performed.

b) DFG-iNOS-Neo

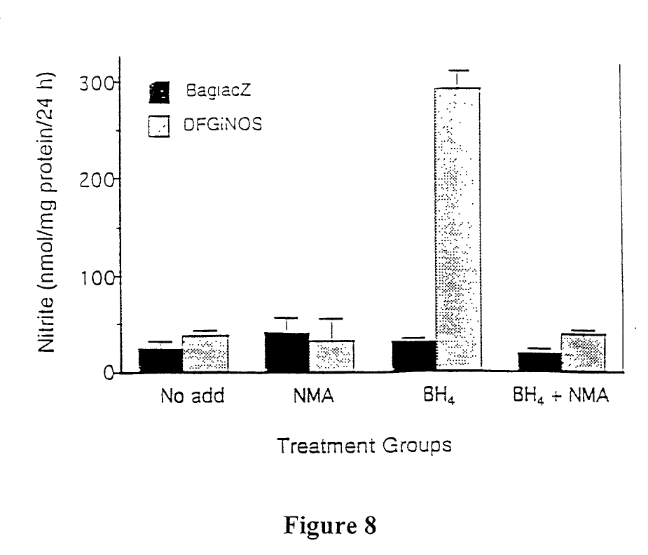

This MFG-iNOS containing retroviral construct comprises a selectable neomycin resistance marker (see FIG. 6 and FIG. 8.) The MFG retroviral vector had been previously engineered to contain an internal ribosome entry site (IRES) followed by a neomycin resistance gene (Neo^{R}) inserted at the 3′ BamHI cloning site of MFG. The IRES sequence allows for the translation of multiple protein products from a single polycistronic mRNA. This MFG-IRES-Neo^{R} plasmid was digested with the restriction enzymes SalI (which cuts approximately 3000 bps upstream of the NcoI cloning site of MFG) and BamHI. The larger fragment containing the majority of the MFG backbone attached to IRES and Neo^{R} was purified. The previously constructed MFG-iNOS vector was also digested with SalI and EcoRI and a 3.7 Kb fragment containing the 5′ portion of the iNOS cDNA was isolated. The 3′ end of the iNOS cDNA was the identical 3′ fragment with the BclI linker used to construct MFG-iNOS. A 3 part ligation with MFG-IRES-Neo^{R}, 5′ SalI-EcoRI fragment containing the 5′ end of the iNOS cDNA, and 3′ iNOS cDNA with the BclI linker was performed. The ligation mixture was then transformed into E. coli and selected for ampicillin resistant transformants. Such a positive transformant with the correctly oriented construct, referred to throughout this specification as DFG-iNOS-Neo or DFG-iNOS, was isolated and a large scale plasmid preparation performed.

c) Ad-iNOS

The large size of the adenoviral genome requires that it be separated into two separate plasmids before recombinant manipulations can be performed. The plasmid carrying the 5′ portion of the genome was employed for the construction of an adenoviral plasmid carrying the iNOS cDNA. The E1 region of the adenoviral genome was previously deleted from this plasmid and in its place, the full-length iNOS cDNA was inserted along with a CMV enhancer/promoter complex. After this plasmid was generated, it was cotransfected with the plasmid carrying the remainder of the adenoviral genome into 293 cells. These cells constitutively express the E1 gene product and are therefore able to package infectious adenoviral particles from E1 deleted constructs. Following transfection. intracellular recombination occurs to generate the full-length adenoviral genome containing the iNOS cDNA. Infectious AdiNOS particles are then generated and released from the 293 cells through a lytic process and the culture supernatant is collected. This supernatant is subjected to sucrose banding to purify and concentrate the AdiNOS viral particles. The virus can be stored at −80° C. for extended periods of time.

d) Control Vectors

The control retroviral vectors MFGlacZ and BaglacZ were previously described (Zitvogel, et al., 1994, Human Gene Ther. 5: 1493-1506; Price, et al., 1987, Proc. Natl. Acad. Sci. USA 84: 156-160) and are shown schematically in FIG. 6. Both constructs carry the β-galactosidase gene while BaglacZ additionally carries the Neo gene. The control adenovirus vector Ad-LacZ carries the β-galactosidase gene.

### 7.1.2. Production of Replication-deficient Retrovirus

a) Primary Porcine Endothelial Cells

The retrovirus constructs of Example Section 13.1.1 are transfected into the CRIP cell packaging line (Danos and Mulligan, 1988, Proc. Natl. Acad. Sci. USA 85: 6460-6464) using a standard calcium phosphate transfection procedure. The viral vector DFG-iNOS-Neo is capable of imparting resistance to the synthetic antibiotic G418. CRIP cells transfected with DFG-iNOS-Neo were selected on the basis of resistance to G418. The CRIP cell line expresses the three viral proteins required for packaging the recombinant viral RNAs into infectious particles. Moreover, the viral particles produced by the CRIP cell line are able to efficiently infect a wide variety of species of mammalian cells including human cells. All retroviral particles produced by this cell line are defective for replication but retain the ability to stably integrate into mammalian cells, thereby transferring an heritable trait to these cells. Virus stocks produced by this method are substantially free of contaminating helper-virus particles and are also non-pathogenic.

b) Sheep Pulmonary Artery Endothelial Cells and Rat Pulmonary Artery Smooth Muscle Cells

As noted above, the DFG-iNOS-Neo plasmid was calcium phosphate transfected into the transient ecotropic packaging cell line BOSC23 (Pear, et al., 1993, Proc. Natl. Acad. Sci. USA 90: 8392-8396). Viral supernatants were collected 72 h after transfection and used to infect CRIP cells (Danos and Mulligan, 1988, Proc. Natl. Acad. Sci. USA 85: 6460-6464) to generate a stable amphotropic producer cell line. CRIP cells were incubated with BOSC23 viral supernatant with 8 μg/ml polybrene (Sigma; St. Louis, Mo.) and then selected with G418 (750 μg/ml, Geneticin, GIBCO BRL; Grand Island, N.Y.). The BOSC23 supernatant had an estimated titer of 10⁵ PFU/ml. Individual G418-resistant CRIP colonies were isolated and screened for nitrite (NO₂⁻) production as an indirect measure of iNOS expression. The colonies generating the highest NO₂⁻ levels were tested for virus production by the number of G418-resistant NIH3T3 colonies following infection with serial dilutions of the CRIP-DFG-iNOS-Neo supernatants. The BAG mobilization assay for replication competent helper virus was performed as previously described (Danos O., 1991, Construction of retroviral packaging cell lines. In: Collins M (ed) Methods in Molecular Biology, Vol. 5: Practical Molecular Virology, Viral Vectors for Gene Expression. Humana Press Inc., Clifton, N.J. pp. 17-27).

### 7.1.3. Cell Culture

a) BOSC23 Cells

BOSC23 cells were grown in DMEM, 10% fetal calf serum, 100 U/ml penicillin, 10 μg/ml streptomycin, and 4 mM glutamine

b) CRIP Cells

CRIP cells were grown in DMEM, 10% fetal calf serum, 50 U/ml penicillin, 50 μg/ml streptomycin, and 10 mM HEPES.

c) Primary Porcine Endothelial Cells

Primary porcine endothelial cells, derived from Yucatan minipig (YPE cells), were isolated as described by Reitman, et al. (1982, Atherosclerosis 43: 119-132) as outlined in Nabel, et al. (1989, Science 244: 1342-1344). Cells are incubated with medium 199 (M199) supplemented with 10% FBS, 2 mM L-glutamine, 50 U/ml penicillin, and 5 ug/ml streptomycin.

d) Sheep Pulmonary Artery Endothelial Cells

Sheep pulmonary artery endothelial cells (SPAEC) were isolated by collagenase digestion and grown in OPTI-MEM I (GIBCO BRL; Grand Island, N.Y.), 10% sheep serum, 100 U/ml penicillin, 100 μg/ml streptomycin, 15 μg/ml endothelial cell growth supplement: (Collaborative Biomedical Products; Bedford, Mass.), and 10 U/ml heparin. At the 2^{nd}-3^{rd} passage, cells were incubated with 1,1′-dioctadeyl-1,3,3,3′,3′-tetramethylindocarbocyanine perchlorate-labeled acetylated low density lipoprotein (DiIAc-LDL, Biomedical Technologies Inc; Staughton, Mass.) and cells preferentially incorporating DiIAcLDL were isolated by fluorescence activated cell sorting (FACstar, Becton Dickinson Immunocytometry; San Jose, Calif.). Subcultures were routinely positive for DiIAc-LDL uptake as well as von Willebrand factor (vWF).

e) Rat Pulmonary Artery Smooth Muscle Cells

Rat pulmonary artery smooth muscle cells (RSMCs) were isolated from left pulmonary artery explants as previously described (Davies and Patton. 1994, J. Cell. Physiol. 159: 399-406). The cells were maintained in DMEM/F12 (1:1 vol), 10% fetal calf serum, 4 mM L-glutamine, 100 U/ml penicillin, and 100 ug/ml streptomycin. The cells had the characteristic spindle shape of smooth muscle cells and were positively identified by indirect immunofluorescence staining for α-actin and smooth muscle-specific myosin. Only early passage RSMCs (passages 3-8) were utilized. All cell lines were grown at 37° C. in a 5% CO, 95% air incubator.

### 7.1.4. Infection of Target Cells

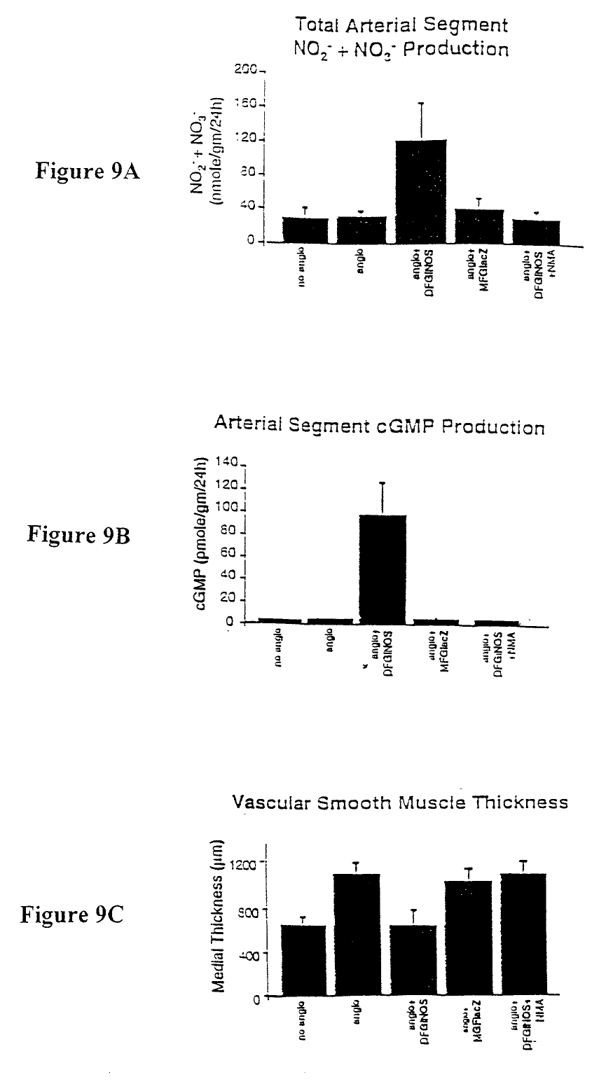

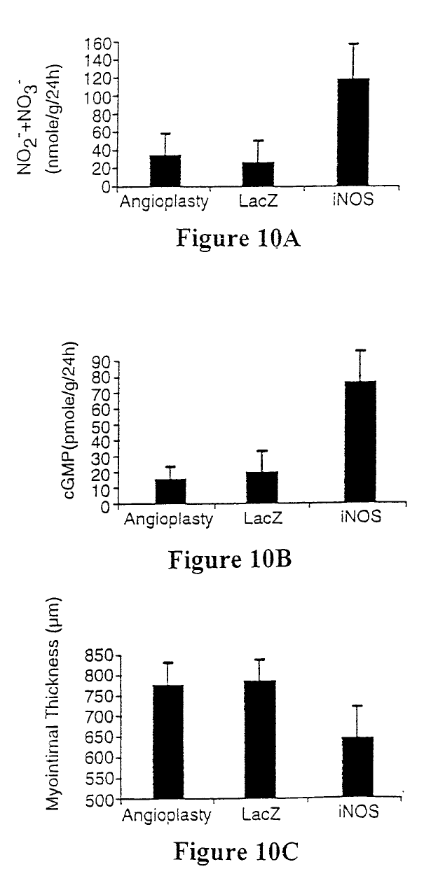

To determine effective infection, endothelial cell nitric oxide production is determined by several methods and compared to endothelial cells infected with control viruses. Nitric oxide produced by the intact cells can be quantified by measuring the release of NO₂⁻+NO₃⁻ into the culture medium. FIGS. 9 and 10 demonstrate successful transfer of iNOS function to endothelial cells using both MFG-iNOS and DFG-iNOS-Neo vectors as evidenced by increased NO₂⁻ production in comparison to uninfected and control virus infected endothelial cells. Enzyme activity within the cells can be measured in cytosolic preparations from cultured cells. iNOS can be distinguished from native cNOS by excluding activity in the membrane fraction, where 70-90% of native cNOS is located. Alternatively, iNOS can be distinguished from native cNOS by determining calcium dependence. Native cNOS is dependent on added calcium while iNOS is not. The presence of iNOS mRNA will be detected by Northern blot analysis. Based on the human iNOS sequence, a set of human specific PCR primers for iNOS has been designed which do not amplify the endothelial cNOS mRNA. iNOS protein is sought by Western blot analysis of cytosolic proteins and immunohistochemistry of intact cells (to localize sites of expression within the cell). Previously characterized human and murine iNOS antibodies, as well as a human cNOS antibody are utilized in Western blot analysis and immunohistochemical techniques. The immunohistochemistry allows for an estimation of the efficiency of infection by calculating the percentage of positive staining endothelial cells. The stability of iNOS expression in the endothelial cells is followed over time through subsequent cell passages. Nitric oxide-induced toxicity will be determined by cellular morphology as well as by ³H-thymidine uptake for DNA synthesis. In vitro toxicity encountered due to excessive nitric oxide production can be controlled by adding inhibitors such as N^{G}-monomethyl-L-arginine (NMA), which competitively inhibits the iNOS enzyme but does not effect gene expression. A second technique for limiting any nitric oxide toxicity is the addition of hemoglobin to the cultures. Hemoglobin rapidly binds and deactivates nitric oxide.

a) Primary Porcine Endothelial Cells

Viral supernatants for both the MFG-iNOS and DFG-iNOS-Neo vectors were used to infect primary porcine endothelial cells in vitro as described by Zwiebel, et al. (1989, Science 249:220-222). Briefly, CRIP producer cells transfected with MFG-iNOS proviral DNA carried no selectable markers. A heterogenous population of CRIP cells, some producing MFG-iNOS virus and some not, was grown and viral supernatants collected to infect endothelial cells. Those CRIP cells transfected with DFG-iNOS-Neo carried a selectable marker (resistance to G418) and a CRIP producer line generating high titer MFG iNOS-Neo viral supernatants was isolated and propagated. Viral supernatants for MFG-iNOS and DFG iNOS-Neo were found to be free of these viruses. These viral supernatants are used to infect endothelial cells in vitro and iNOS activity assayed at 48-72 hours after infection as described below (see FIGS. 9 and 10).

b) Sheep Pulmonary Artery Endothelial Cells and Rat Pulmonary Artery Smooth Muscle Cells

SPAECs and RSMCs at 50% confluence were infected for 8 h with either DFG-iNOS-Neo or BaglacZ viral supernatant with 8 μg/ml polybrene and selected in G418 (500-750 μg/ml). Following selection, >90% of BaglacZ infected SPAECs and RSMCs possessed β-galactosidase activity by X-gal staining. All cells were cultured in 0.5 mM N^{G}-monomethyl-L-arginine (L-NMA) to inhibit NO synthesis until 24 h prior to experimentation. A subgroup of SPAEC/DFG-iNOS-Neo was cultured without L-NMA for >14 d to study the effects of sustained high level NO production. L-NMA administration did not alter cell growth or morphology.

### 7.1.5. RNA Isolation and Northern Blot Analysis

Total cellular RNA was isolated from uninfected, BaglacZ. and DFG-iNOS-Neo SPAECs and RSMCs as well as SPAEC treated for 6 h with 1 mM N-acetyl-penicillamine (NAP) or S-nitroso-N-acetyl-penicillamine (SNAP, Sigma; St. Louis, Mo.) using RNAzol B as previously described (Chomczynski and Sacchi, 1987, Anal. Biochem. 162:156-159). Aliquots (20 μg) of RNA were electrophoresed on a 0.9% agarose gel and blotted to GeneScreen (DuPontNEN; Boston, Mass.). After prehybridization, the membranes were hybridized to a DNA probe as described (Geller, et al., 1993, Proc. Natl. Acad. Sci. USA 90:522-526). A 2.3 kb human iNOS cDNA fragment served as the iNOS probe while a 4.1 kb human endothelial cNOS cDNA fragment served as the ecNOS probe. 18S rRNA served as a control for relative RNA loading.

### 7.1.6. Cell Lysate Preparation

Uninfected, BaglacZ, and DFG-iNOS-Neo infected SPAECs were washed and resuspended in protease inhibitor buffer (20 mM TES pH 7.4, 2 mM DTT, 10% glycerol, 25 μg/ml Antipain, 25 μg/ml Aprotinin, 25 μg/ml Leupeptin, 25 μg/ml Chymostatin, 50 μM Phenanthroline, 10 μg/ml Pepstatin A) supplemented with 10 μM FMN, 10 μM FAD, and 5 μM BH₄. The cells were lysed by three freeze-thaw cycles, and the cytosolic fraction was isolated by centrifugation at 100,000×g for 60 min at 4° C. as previously described (Luss, et al., 1994, Biochem. Biophys. Res. Comm. 204-2: 635-640). Protein concentrations were measured with the BCA protein assay (Pierce; Rockford, Ill.). For whole cell preparations, a similar procedure was performed without the centrifugation step.

### 7.1.7 Western Blot Analysis

Cell cytosols (100μg) were electrophoresed through an 8% SDS-polyacrylamide gel and transferred to nitrocellulose membranes (Schleicher Schuell; Keene, N.H.) as described (Laemmli, 1970, Nature 227: 680-685). Membranes were blocked with 5% milk/phosphate buffered saline/0.1% Tween-20 and hybridized with a monoclonal antimouse macrophage iNOS antibody (1:2000 dilution, Transduction Laboratories: Lexington, Ky.) that detects human iNOS, followed by a goat anti-mouse IgG linked to horseradish peroxidase (Schleicher & Schuell; Keene, N.H.). Human hepatocyte cytosol isolated 14 h following stimulation with 5 U/ml human IL1β (Cistron, Pine Brook, N.J.), 500 U/ml human TNFα (Genzyme), 100 U/ml human IFFγ (Amgen), and 10 μg/ml LPS (Escherichia coli 0111:B4, Sigma: St. Louis, Mo.) served as a positive control. The membrane was developed with ECL reagents (DuPont-NEN; Boston, Mass.) and exposed to Kodak X-Omat film for 1-20 minutes at room temperature. For ecNOS, 25 μg of whole cell preparations were electrophoresed. The primary antibody was monoclonal murine IgG against human ecNOS (1:2000 dilution, Transduction Laboratories; Lexington, Ky.) that detects bovine ecNOS.

### 7.1.8 Assay for NO₂⁻ and NO₃⁻ Production

The direct iNOS enzyme assay measures the conversion of [³H]-arginine to [³H]-citrulline, as described (Bredt, et al., 1991, Nature 351:714-718).

NO₂⁻+NO₃⁻ levels are measured in the culture supernatants by an automated procedure based on the Griess reaction (Green, et al., 1982, Anal. Biochem. 126: 131-137). Briefly, uninfected, BaglacZ, and DFG-iNOS-Neo SPAECs and RSMCs were passaged to 6 well plates at near confluency in the presence of 0.5 mM L-NMA. Culture medium was then replaced with fresh medium and the cells were cultured for an additional 24 h at which time the supernatants were assayed for accumulated NO₂⁻ using the Griess reaction (see also, Geller, et al., 1993, Proc. Natl. Acad. Sci. USA 90: 522-526). Measurements were also performed in the presence of L-NMA (0.5 mM) and/or BH₄ (100 μM). The cells in each well were then lysed with 1% Triton-X100/25 mM Tris-phosphate/2 mM EDTA/10% glycerol and the protein concentration was quantified with the BCA protein assay. Some supernatants were also assessed for total NO₂⁻ and NO₃⁻ levels with a standardized HPLC assay utilizing an in-line column containing copper-coated cadmium as previously described (Billiar, et al., 1989, J. Exp. Med. 169: 1467-1472).

### 7.1.9 Arterial Organ Culture

a) Porcine Femoral Arteries

Femoral arteries were collected from anesthetized (sodium pentobarbital, 4 mg/kg) domestic pigs through bilateral groin incisions and immediately immersed into sterile phosphate buffered saline. The adventitia was gently dissected free and some vessels were uniformly injured with a 4-French balloon catheter inflated to 10 atmospheres for 30 sec. All arteries were opened along the long axis, divided into 1 cm long sections, and cultured in DMEM, 20% FCS, 100 U/ml penicillin, 100 μg/ml streptomycin, and 4 mM L-glutamine at 37° C. as previously described (Takeshita, et al., 1994, J. Clin. Invest. 93: 652-661). On culture day, some arterial segments were incubated with 2 mls of either DFG-iNOS-Neo or MFGlacZ viral supernatant (both titers˜10⁶ PFU/ml) supplemented with polybrene (8 μg/ml) for 6 h. Following infection, the vessels were transferred to fresh culture dishes to remove any explanted cells and were maintained in organ culture for a total of 14 days with daily media changes. After initial observations that NO₂⁻+NO₃⁻ release from the DFG-iNOS-Neo transfected vessels was BH₄-dependent, BH₄ (100 μM) was supplemented on a daily basis to all the cultures. L-NMA (0.5 mM) was added to some vessel preparations. On day 14, culture supernatants were collected for NO₂⁻+NO₃⁻ and cGMP determinations. cGMP levels were measured with a commercial radioimmunoassay (NEN: Boston, Mass.).

To evaluate efficiency of MFGlacZ infection, vessel segments were fixed in 0.5% glutaraldehyde for 30 min and stained for β-galactosidase activity with X-gal. DFG-iNOS-Neo segments were fixed in 2% paraformaldehyde for 1 h at 4° C. and cryoprotected in 30% sucrose overnight at 4° C. Vessels were then quick frozen with HistoFreeze™-2000 (Fisher; Pittsburgh, Pa.) and 5 μm cryosections cut. Sections were mounted on glass slides, permeabilized with 2% paraformaldehyde/0.1% Triton-X100, blocked with 5 % goat serum, and then incubated with the primary monoclonal antimurine iNOS antibody previously used for Western blot analysis. The antibody staining was visualized with immunoperoxidase. To measure myointimal thickness, semi-serial sections were incubated for 60 min with rhodamine phalloidan (Molecular Probes, Inc.; Eugene, Oreg.), which binds to actin. These preparations were visualized with indirect fluorescence microscopy (Nikon, FA) and recorded by a Sony DXC 930 camera linked to a computer. The myointimal thickness was quantified with the Optimas program (Optimal Corp.; Seattle, Wash.) at 25 random sites along the length of each vessel segment and calculated as the mean of all the measurements.

Some vessels were homogenized with a polytron and the RNA was extracted with RNAzol as described above. First strand cDNA synthesis was performed on 500 ng of total RNA in a volume of 10 μl with 50 mM Tris-HCl (pH 8.3), 75 mM KCl, 3 mM MgCl₂, 1.0 mM dNTPs, 10 mM DL-dithithreitol, 10 U human placental RNAase inhibitor, and 200 U MMLV reverse transcriptase (GIBCO; Gaithersburg, Md.) at 37° C. for 60 min. cDNAs (100 ng) were combined in 50 μl in 10 mM Tris-HCI (pH 8.3), 50 mM KCl, 200 μM dNTP, 1.5 mM MgCl₂, 100 pM each PCR primer, and 1.25 U Taq DNA polymerase (Perkin-Elmer Cetus: Norfolk, Conn.) and PCR amplification carried out with denaturation at 94° C. for 1 min. annealing at 57° C. for 2 min and elongation at 72° C. for 3 min for 40 cycles. The iNOS oligonucleotide primers specifically recognize the human hepatocyte iNOS cDNA sequence and do not detect rodent sequences. The 18 bp 5′ primer spanned from bp 3376-3393 of SEQ ID NO:1 of the iNOS cDNA and the 3′ primer spanned from bp 3674-3691 of SEQ ID NO:1. The predicted PCR product is 316 bps. PCR amplification for Neo mRNA, unique to the DFG-iNOS-Neo virus, was performed as another marker for expression of the iNOS transgene (Neo PCR product=728 bp). RT PCR for β-actin message served as a control. The β-actin PCR product measures 652 bps. PCR products were visualized on a 1.5% agarose gel.

b) Human Coronary Arteries

Human coronary arteries were extracted from the extirpated hearts of patients undergoing cardiac transplantation. Immediately upon extirpation, the left anterior descending coronary artery was sharply dissected from the left ventricle. The anterior and posterior tibial arteries were obtained from patients undergoing lower extremity amputations immediately following the amputation. All vessels were immediately placed in normal saline solution. A 2 or 4 French catheter was placed into the vessel segment and inflated with saline from a 1 cc syringe with the balloon remaining inflated for 30 seconds. Under sterile conditions, the adventitial tissue was sharply dissected from the vascular segments. The vessels were then divided into 1 cm sections for placement into the organ culture system. The organ culture system contained DMEM supplemented with 20% fetal calf serum, 2mM L-glutamine, 100 unit/ml penicillin and 100 μg/ml streptomycin.

Once placed into the organ culture the media was changed daily. On day 5, the vessels are infected with DFG-iNOS-Neo. One ml of the retroviral supernatant containing 10⁶ CFU/ml was added to each vessel being transfected. On the following day, the viral supernatant was removed and the routine media solution added to the organ bath. Daily media changes are again performed with 100 μM BH₄ being added to each well. On day 14, the media were collected for total nitrite and nitrate as well as cGMP measurements. The vessels are then frozen for histologic analysis.

To examine the thickness of the medial layer following the arterial injury and ensuing 14 days in culture, the vessels are sectioned into 5 micron sections. The vessels are washed twice with 1% PBS solution and stained with rhodamine phalloidin for 60 min. The sections are washed again with 1% PBS and cover slipped. The segments were prepared and examined as described in section a).

### 7.1.10 Statistical Analysis

Values for NOR, NOR+NO3 , cGMP, and myointimal thickness are expressed as means+SD. The significance of differences was determined using the ANOVA test. Statistical significance was established at a p value <0.01.

### 7.1.11. In Situ ADiNOS Infection

Rats were anesthetized with nembutal and the left common carotid artery was exposed through a collar incision. A 2 French Fogarty catheter was introduced through the left external carotid artery into the common carotid and the balloon was inflated to create a vascular injury. Following balloon injury, AdiNOS or AdlacZ at a titer of 10⁷ pfu/ml was infused into the common carotid artery through the external carotid and allowed to incubate for a 60 minute period. After the incubation period, the virus was evacuated and the external carotid artery ligated and the flow was reestablished through the common carotid artery. The collar incision was closed and the animal revived. Rats were housed for a total of 14 days at which time they were sacrificed and both carotid arteries were collected for molecular and histological studies.

Domestic pigs will be anesthetized with sodium pentobarbital and bilateral iliac arteries will be exposed through a low midline abdominal incision. A small arteriotomy will be created through which a 4 French Fogarty catheter will be introduced. Inflation of the Fogarty will be used to create a vascular injury. AdiNOS or AdlacZ (10⁷ pfu/ml) will be instilled into an isolated segment of the iliac artery and permitted to incubate for 60 minutes. After the incubation period, the virus will be evacuated, the arteriotomy repaired, and flow reestablished. One iliac vessel will serve as the experimental side while the contralateral will serve as the control. Pigs will be housed for periods of time varying between 1, 3 and 6 weeks. At the end of these time periods, the pigs will be sacrificed and bilateral iliac arteries will be collected for molecular and histological studies.

For histologic evaluation, the vessels are fixed in paraformaldehyde and sucrose and then cryopreserved. Following sectioning, tissues are stained with hematoxylin and eosin. Intimal and medial thicknesses is quantitated using computer imaging programs. LacZ staining is performed using X-gal to detect β-galactosidase activity. Immunostaining for iNOS will be performed with a polyclonal iNOS antibody against murine iNOS that detects human iNOS followed by treatment with a secondary antibody complexed to horseradish peroxidase. Cellular proliferation will be quantitated with bromodeoxyuridine (BrdU) or by immunostaining with an antibody directed against proliferating cell nuclear antigen (PCNA).

### 7.2. Results

### 7.2.1 Generation of Viral Vectors

a) MFG-iNOS

The human hepatocyte iNOS cDNA was inserted into the NcoI and BamHI cloning sites of the retroviral vector MFG as shown in FIG. 6 and FIG. 7. Viral supernatants for MFG-iNOS were used to infect endothelial cells in vitro and iNOS activity assayed at 48-72 hours after infection (FIGS. 9).

b) DFG-iNOS-Neo

The human hepatocyte iNOS cDNA was inserted into the NcoI and BamHI cloning sites of the retroviral vector MFG as shown in FIG. 6 and FIG. 8. Viral supernatants for MFG-iNOS were used to infect endothelial cells in vitro and iNOS activity assayed at 48-72 hours after infection (FIGS. 10).

Functional expression of DFG-iNOS-Neo was tested in BOSC23 by assaying for NO₂⁻ production following transfection with DFG-iNOS-Neo plasmid DNA. BOSC23 transfected with DFG-iNOS-Neo produced NO₂⁻ levels of 35.3+2.6 uM/48 h vs. 0.7+0.2 for cells transfected with MFGlacZ DNA (p<0.01). BOSC23 supernatants were used to infect CRIP cells to generate a stable amphotropic producer cell line. Following infection and G418 selection, a mixed CRIP/DFG-iNOS-Neo cell population produced high levels of NO₂⁻ (50.9+2.8 uM/24 h) when BH₄ was supplemented. In the absence of exogenous BH₄, however, NO₂⁻ levels of only 4.7+1.0 uM/24 h were measured (p<0.01). BH₄ is known to be an essential cofactor for all NOS enzymes, necessary in part for maintaining the active structural configuration of the enzyme. A G418-resistant CRIP clone was found to produce DFG-iNOS-Neo virus at a titer of 5×10⁶ PFU/ml and exhibited NO₂⁻ production of approximately 50 nmol/10⁶ cells/24 h in the presence of BH₄. Viral supernatant from this clone tested free of replication competent virus and was used in all subsequent experiments. No difference was detected in viral titers from BOSC23/DFG-iNOS-Neo grown with or without the NOS inhibitor L-NMA (both approximately 10⁵ PFU/ml). These results demonstrate that a functional iNOS retroviral vector can be generated and that certain cells may lack the BH₄ synthesis required to support NO synthesis wherein BH₄ supplementation was found to be adequate to optimize iNOS activity.

c) AdiNOS

AdiNOS supernatant was tested on a variety of cell types for the ability to infect and transfer iNOS expression to naive cells. These cells include human smooth muscle cells, endothelial cells, and hepatocellular cell lines as well as rat SMCs and primary hepatocytes. All cells were successfully infected with AdiNOS with varying levels of efficiency. High levels of iNOS expression and nitric oxide synthesis were detected for all the cells tested, with the greatest nitric oxide synthesis occurring in hepatocytes. These results demonstrated that AdiNOS was a functional viral vector that successfully transfer iNOS gene expression into a variety of cell types.

### 7.2.2 Transfer and Expression of Human iNOS in Vascular Endothelial Cells

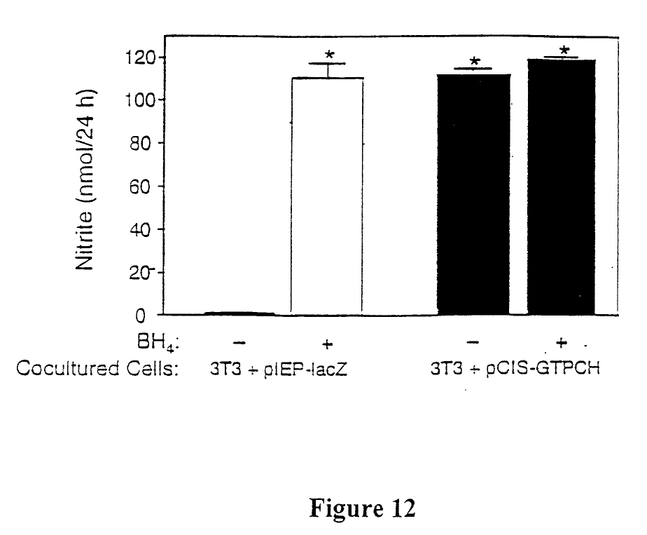

Sheep pulmonary arterial endothelial cells were infected with the high titer DFG-iNOS-Neo supernatant and selected in G418. By Northern blot analysis (FIG. 12), high levels of iNOS mRNA were found in SPAEC/DFG-iNOS-Neo but not in either uninfected or BaglacZ control groups. The iNOS mRNA in SPAEC/DFG-iNOS-Neo migrated at 7.5 kb, distinct from the 4.5 kb size of endogenous human hepatocyte iNOS mRNA and corresponded to the expected size of the polycistronic DFG-iNOS-Neo retroviral transcript. No 4.5 kb signal was detected in any of the SPAEC groups. Stimulation of SPAEC with cytokine combinations effective in inducing iNOS expression in other cell types failed to yield detectable levels of sheep iNOS mRNA. Western blot analysis of SPAEC lysates (FIG. 13) demonstrated the presence of iNOS protein in SPAEC/DFG-iNOS-Neo cytosolic preparations, similar in mobility to endogenous human iNOS protein in cytosol from cytokine treated human hepatocytes.

NO₂⁻ production (representing 40% of total nitrogen oxide metabolites) by uninfected, BaglacZ, and DFG-iNOS-Neo infected SPAEC is summarized in FIG. 14. SPAEC/DFG-iNOS-Neo produced 155.0+10.7 nmol/mg protein/24 h as compared to 5.5+1.1 by SPAEC-BaglacZ and 6.4+1.1 by uninfected cells (p<0.01). Nitrogen oxide synthesis by these cells was inhibited by the addition of L-NMA to the culture medium. Supplemental BH₄ did not significantly increase NO₂⁻ production, unlike the CRIP cells that had been dependent on added cofactor. The results indicate these proliferating vascular endothelial cells can express and support a functional human iNOS enzyme and that such cells could produce sufficient BH₄ to support NO synthesis activity.

The expression of ecNOS is important to normal endothelial function. Therefore, the effect of sustained supraphysiologic NO synthesis by iNOS on endogenous ecNOS expression in these endothelial cells was also examined. Northern blot analysis (FIG. 15A) revealed that steady-state ecNOS mRNA levels were not significantly altered in SPAEC-DFGiNOS maintained with or without L-NMA for greater than 14 d as compared to native SPAEC. Similarly, exposure of SPAEC to the exogenous NO donor SNAP (1 mM) for 6 h resulted in a 1.5 fold increase in ecNOS mRNA versus SPAEC treated with the parent compound. Levels of ecNOS protein did not vary between protein isolated from whole cell preparations of uninfected SPAEC, SPAEC-DFGiNOS maintained in L-NMA, or SPAEC-DFGiNOS grown in the absence of L-NMA (FIG. 15B). ecNOS protein was not detectable in the cytosolic fractions from these groups. Thus, stable expression of iNOS had minimal effects on ecNOS mRNA and protein levels.

### 7.2.3 Transfer and Expression of Human iNOS in Vascular Smooth Muscle Cells

The ability of smooth muscle cells to support a foreign iNOS enzyme was also examined. In contrast to SPAEC. RSMC transduced with DFG-iNOS-Neo produced high levels of nitrogen oxides only when BH₄ was supplemented (FIG. 16A). In the presence of exogenous BH₄, RSMC-DFGiNOS NO synthesis increased almost 10 fold but in the absence of BH₄, little NO₂⁻ could be measured. Northern blot analysis revealed that retroviral iNOS expression, as marked by the characteristic 7.5 kb viral iNOS transcript, was independent of BH₄ availability (FIG. 16B).

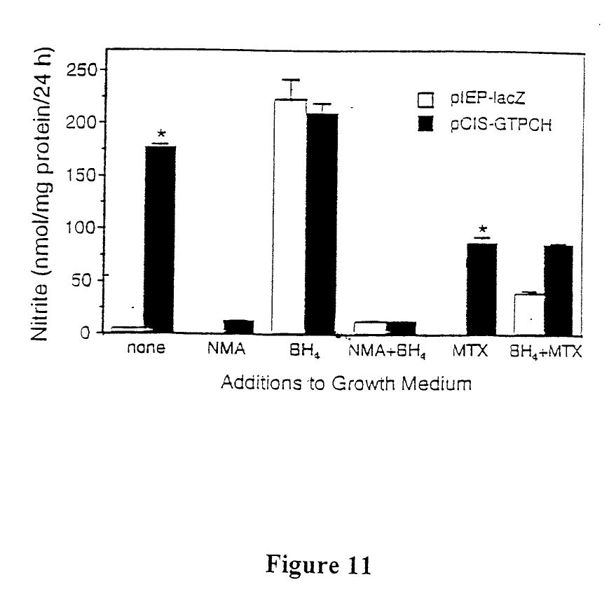

FIG. 11 depicts results of a pCIS-iNOS/lipofectamine transfection targeting vascular smooth muscle cells. Significant nitrite production is detected for pCIS-iNOS transfected vascular smooth muscle cells in the absence, but not the presence, of N^{G}-monoethylarginine. Additionally, no nitrite production was detected upon transfection with a control plasmid (pSV-lacZ), and a plasmid-less control with or without the addition of liposomes. As discussed throughout the specification, this method of targeting endothelial and/or vascular smooth muscle cells is especially preferred for in situ transfection of target cells lining the arterial lumen.

In vitro infection of RSMC with AdiNOS at a multiplicity of infection (MOI) of 10 resulted in significant nitrite accumulation (see Table 1) as compared to cells infected with AdlacZ. In the presence of supplemental BH₄, the amount of nitrite generated doubled and demonstrates a partial dependence of these cells on exogenous cofactor to support iNOS activity. iNOS expression and NO synthesis in RSMC following AdiNOS infection resulted in a significant reduction in SMC DNA synthesis and proliferation as measured by ³H-thymidine incorporation (Table 1). Proliferation was reduced by nearly 60%. When the RSMCs were provided with supplemental BH₄, NO synthesis doubled and proliferation was further inhibited by 75%. Inhibition of NO synthesis by the arginine analog L-NG-monomethyl-arginine was only partial and resulted in a partial recovery of cellular proliferation. Infection of these cells with the control AdlacZ virus did not result in any nitric oxide synthesis and no effect on ³H-thymidine incorporation was detected.

### 7.2.4 Transfer and Expression of Human iNOS to Injured Femoral Arteries In Vitro

iNOS gene transfer to arterial vessels was evaluated in vitro with intact porcine femoral arteries in organ culture. Following balloon catheter-induced vascular injury and viral infection five days after injury, arterial segments infected with DFG-iNOS-Neo released 3-4 fold more NO₂⁻+NO₃⁻ vs. uninjured vessels or MFGlacZ-infected segments (Table 2) as measured on culture day 14. More dramatically, cGMP release by DFG-iNOS-Neo infected arteries increased by 15 fold over that measured in either uninjured or injured control vessel segments. BH₄ was provided to the organ cultures on a daily basis because initial results indicated nitrogen oxide and cGMP release was dependent on cofactor supplementation. Inclusion of L-NMA in the culture media inhibited both NO₂⁻+NO₃⁻ and cGMP release.

Staining for β-galactosidase or iNOS in the infected arterial segments showed an estimated infection efficiency of 0.5-1%. The majority of cells expressing either enzyme were found to be located in the superficial neointimal region (FIG. 20). Transgene expression was further confirmed by RT-PCR amplification for human iNOS message. The predicted 316 bp iNOS PCR product was strongly detected only in DFG-iNOS-Neo infected vessel segments. A very low level of iNOS mRNA was detected in MFGlacZ infected vessels. While the iNOS PCR primers did not amplify rodent iNOS sequences, there may be some cross-reactivity with porcine iNOS sequences. Detectable iNOS expression by PCR amplification in control vessels may reflect a low level induction secondary to balloon-catheter injury. However, amplification for Neo sequences (FIG. 17), unique to the DFG-iNOS-Neo retrovirus, revealed expression solely in the DFG-iNOS-Neo infected vessels and provides additional confirmation of expression of the transferred genes.

FIG. 18(A-C) and FIG. 19(A-C) show data generated from in vitro cultured porcine arteries infected with DFG-iNOS-Neo (FIG. 18) as well as diseased human coronary and tibial arteries infected with DFG-iNOS-Neo (FIG. 19). The control construct in both FIG. 18 and FIG. 19 was MFGLacZ. FIG. 18A and 19A show that total nitrite production was significantly elevated in the vessels infected with DFG-iNOS-Neo as compared to vessels undergoing angioplasty alone or infected with MFGLacZ. The elevation in total NO production was abrogated by adding the NO inhibitor LNMA. FIG. 18B and FIG. 19B show that cyclic GMP levels were also significantly elevated in infected arterial segments when compared to uninfected segments and segments infected with the control retrovirus MFG-lacZ. FIG. 18C and FIG. 19C shows that arterial injury resulted in a significant increase in the total thickness of the medial layer. Infection with the DFG-iNOS-Neo vector resulted in the inhibition of this proliferative process. The medial thickness in vessels infected with DFG-iNOS-Neo and grown in L-NMA or vessels infected with MFG-lacZ were similar to the angioplasty control segments.

### 7.2.5 Transfer and Expression of Human iNOS in Injured Rat Carotid Arteries In Vivo

In vivo transfer of iNOS into the rat carotid artery injury model was performed with AdiNOS. Control animals included animals subjected to carotid artery injury alone or subjected to injury followed with infection with AdlacZ control virus. Histologic examination of the experimental carotid arteries 14 days following injury and gene transfer revealed that arterial injury alone resulted in marked intimal hyperplasia with a neointima measuring approximately twice the width of the medial layer. Animals treated with the control AdlacZ virus (FIG. 21) still responded to arterial injury with the formation of a thick neointima that resembled animals subjected to injury alone. However, the carotid arteries that were treated with AdiNOS demonstrated a complete inhibition of this proliferative process with no evidence of neointima formation (FIG. 212). These carotid arteries resembled uninjured arteries. The effect of iNOS gene transfer on myointima proliferation in response to injury measured either an in vitro or in siru assay is summarized numerically in FIG. 19 (in vitro and infected with DFG-iNOS-Neo) and FIG. 21A and FIG. 21B (in situ and infected with Ad-iNOS). FIG. 18C shows that balloon catheter injury resulted in a significant increase in myointimal thickness, as determined by rhodamine phalloidan staining in both injury alone or injury followed by infection with MFGlacZ. In contrast, the proliferative response to balloon injury in arteries subsequently infected with DFG-iNOS-Neo was markedly attenuated and essentially indistinguishable from uninjured vessels. The inhibitory effect of DFG-iNOS-Neo infection on myointimal thickening was completely abrogated by L-NMA administration. indicating the effect was dependent on NO synthesis.

Similar results were obtained by direct in situ infection of porcine arterial vascular cells with Ad-iNOS and Ad-LacZ. Subsequent to mechanical injury to a porcine arterial segment either Ad-iNOS and Ad-LacZ were transferred to intimal vascular cells at the site of catheterization. FIG. 21A and 21B clearly show a marked reduction in myointimal hyperplasia within in situ infected Ad-iNOS arterial segments in comparison to in situ infected Ad-LacZ arterial segments. These results show successful reduction in myointimal hypertrophy following balloon-catheter induced arterial injury with human iNOS gene transfer despite low gene transfer efficiency.

### 7.2.6 In Vitro Requirement for Tetrahydrobiopterin (BH₄) for Optimal iNOS Enzyme Activity

The primary target for iNOS gene delivery in the vasculature will be the smooth muscle cells exposed during vascular injury. These cells have been shown to have at least a 50% dependence on supplemental exogenous BH₄ to support maximal iNOS activity following iNOS gene transfer using liposomes, retroviral vectors, or adenoviral vectors. SMCs do not normally express the rate-limiting enzyme required for BH₄ biosynthesis, GTP cyclohydrolase I (GTPCH). This cofactor is essential for the activity of all the NOS enzymes and NO synthesis cannot occur in its absence. The partial iNOS activity detected in SMCs engineered to express iNOS is due to the presence of a small amount of BH₄ or its precursors in the culture medium that can be utilized by the cells. A method to supplement BH₄ in vivo may be required to maximize the amount of nitric oxide that can be synthesized following iNOS gene delivery. The ability of delivery of the GTPCH gene to cells lacking this enzyme activity to support iNOS activity was examined in NIH3T3 cells. These cells, like SMCs, lack GTPCH enzyme expression and cannot synthesize BH₄. Transfer of GTPCH into NIH3T3 and RSMC using liposomes resulted in the high levels of GTPCH enzymatic activity as well as high levels of intracellular biopterins. Cells transfected with a control expression plasmid containing the lacZ gene did not demonstrate any evidence of GTPCH activity. Transfection of NIH3T3 cells previously engineered to express human iNOS (3T3-iNOS) with the GTPCH gene resulted in maximal levels of nitric oxide synthesis similar to that achieved with exogenous BH₄ supplementation. Low efficiency GTPCH gene transfer could maximally support iNOS activity in these cells and indicates that BH₄ synthesized in a few cells can be transported between cells. In addition, GTPCH and iNOS enzyme activities do not have to coexist within the same cell for nitric oxide synthesis to be supported. These data are discussed at greater length in Example Section 8 along with data presented in Table 3 and FIGS. 22-24.

### 7.3 Discussion

Example Section 7 supports the basis of the disclosure and claims of the present invention: low level transfer of the iNOS gene or a biologically active fragment thereof to the vascular endothelial cells or smooth muscle cells of an arterial vessel provides prophylactic or therapeutic relief from arterial injury. Example Section 7 data shows that DFG-iNOS-Neo infected SPAEC and RSMC support iNOS gene transfer and expression. Injured porcine and human arterial segments cultured in vitro and infected with the iNOS gene show, despite low level transfer, that the effects of mechanical injury to an artery are prevented or overcome by iNOS expression in and around infected vascular cells.

Example Section 7 also supports a novel method of testing gene transfer, expression and therapeutic effects in regard to diseased human arteries. This method is exemplified in this specification by disclosing steps for removing and culturing in vitro diseased human or porcine arterial segments, transferring the iNOS gene to vascular cells of the diseased arterial segment, and measuring the inverse correlation between post infection iNOS expression within vascular cells of the diseased arterial segment and a concomitant decrease in myointimal thickness over time.

Example 7 also presents support for direct in situ transfer of iNOS to vascular endothelial and smooth muscle cells of an arterial vessel injured by balloon catheterization, expression of iNOS within these targeted cells, and an concomitant decrease in myointimal hyperplasia associated with the mechanical injury.

Therefore, Example Section 7 presents numerous tiers of support for the disclosure and claims of the present invention. Cultured vascular endothelial and smooth muscle cells are appropriate targets for infection by iNOS-based viral and non-viral constructs, these constructs are expressed within the target cells and are expressed at levels whereby therapeutic effects may be imparted. Such therapeutic effects are shown in studies where diseased porcine (via mechanical injury) or human (utilizing atherosclerotic vessels obtained from patients during the course of surgery) arterial segments are cultured in vitro, infected with iNOS-based viral or non-viral constructs, and the effects of subsequent iNOS expression is show to curtail effects associated with the injured arterial segment. Finally, evidence from in situ applications show that direct in situ transfer of an iNOS-based construct will result in iNOS expression within the target cells such that the previously injured arterial segment will show a measurable decrease in myointimal hyperplasia.

### 8. Example

### Treatment of Vascular Occlusive Disease

### Co-infection with GTP Cyclohydrolase I

### 8.1. Materials and Methods

Plasmids

The human GTPCH cDNA was cloned by PCR amplification using primers designed based on the sequence of human GCH-1. (Togari, et al., 1992, Biochem. Biophys. Res. Comm. 187: 359-365). It was subcloned into the expression plasmid pCIS. The resultant plasmid pCIS-GTPCH was shown to be a functional expression plasmid. The control expression plasmid pIEP-lacZ contains the cDNA for β-galactosidase (provided by P. Robbins, Univ. of Pittsburgh).

Cell Culture

NIH3T3 cells were cultured in DMEM supplemented with 10% calf serum, 100 U/ml penicillin. 100 μg/ml streptomycin, 4 mM L-glutamine, and 10 μM HEPE buffer in a 95% air/5% CO₂ incubator at 37° C. 3T3-iNOS are NIH3T3 engineered to stably express human iNOS as previously described (Tzeng, et al., 1995, Proc. Natl. Acad. Sci. USA: 92:11771-11775). In brief, NIH3T3 cells were infected with the DFGiNOS retrovirus and were then selected in the synthetic neomycin G418 to yield a population of cells all expressing human iNOS. 3T3 cells lack GTPCH activity and are BH₄ deficient. Abundant levels of iNOS protein are expressed in these cells but NO synthesis can not be detected until exogenous BH₄ is provided in the culture medium. Rat aortic smooth muscle cells were cultured from thoracic aorta explants as described (Davies, et al., 1994, J. Cell Physiol.: 159:399406) and used between passages 2-8. RSMC were maintained in DMENI(low glucose)/F12 (1:1 vol) with 10% fetal calf serum, 100 U/ml penicillin. 100 μg/ml streptomycin and 4 mM L-glutamine.

Liposome Transfection

Cells were passaged to 6 well plates at a density of 1×10⁵ cells/well 24 h prior to transfection. For 3T3 cells, each well of cells was transfected with a mixture of 1 μg plasmid DNA and 6 μg of Lipofectamine (GIBCO) in OPTIMEM-I media for 5 h. For RSMC, a ratio of 1 μg DNA to 7 μl of Lipofectamine was used. Following the incubation period, the transfection solution was removed and normal growth medium replaced. The transfection efficiency was estimated by X-gal staining of pIEP-lacZ transfected cells. All studies were performed at 24-72 h post-transfection.

RNA Isolation and Northern Blot Analysis

Total cellular RNA was collected using RNAzol B from 3T3-iNOS and RSMC transfected with pCIS-GTPCH or pIEP-lacZ 72 h post-transfection. RNA samples (20 μg) were electrophoresed on a 0.9% agarose gel and blotted to GeneScreen (DuPont-NEN). After prehybridization, the membranes were hybridized to a DNA probe as described (Geller, et al., 1993, Proc. Natl. Acad. Sci. USA 90: 522-526). An 800 bp human GTPCH cDNA fragment served as the probe. The positive control for human GTPCH was RNA isolated from human hepatocytes which express GTPCH constitutively. 18S rRNA was used as a control for relative RNA loading.

Measurements of GTPCH Enzymatic Activity and Intracellular Biopterins

Forty-eight hours after transfection, cells were trypsinized and collected for GTPCH enzyme activity measurements. Trypsin was inactivated by fetal calf serum and the cells were washed with Hanks buffer. The cells were then lysed and cytosolic GTPCH activity was determined as previously described (Hatakeyama, et al., 1989, J. Biol. Chem. 264: 21660-21664). For total intracellular biopterin measurements, cells were treated for 60 min with 0.2 N perchloric acid at 0° C. in the dark. The supernatants were collected and tested for total biopterins (BH₄ +BH₂, +biopterin) as previously described (Fukushiam, et al., 1983, Anal. Biochem. 132: 6-13). The cells were lysed with 0.1N NaOH and protein concentrations were measured using the BCA protein assay (Pierce). Serial dilutions of bovine serum albumin served as the standards.

Assay for NO₂− Production

Twenty four hours following transfection, the cell medium was then replaced with fresh medium and the cells were cultured for an additional 24 h at which time the supernatants were assayed for accumulated NO₂ using the Griess reaction (Geller, et al., 1993, Proc. Natl. Acad. Sci. USA: 90:522-526). Measurements were also performed in the presence of L-NMA (1 mM), BH₄ (100 μM), and methotrexate (MTX 12.5 μM). The cells were lysed with 0.1 M NaOH and the protein concentration was quantified with the BCA protein assay. To assess the requirement for compression of GTPCH and iNOS in the same cell, 3T3 cells were transfected with either pIEPlacZ or pCIS-GTPCH. After the 5 h transfection period, the medium was changed and the cells were overlaid with 1×10⁵ cells/well of either 3T3 or 3T3-iNOS. Cells were allowed to attach overnight and NO₂− levels were measured 24 h later.

Statistical Analysis

Values for GTPCH activity, intracellular biopterin levels, and NO, are expressed as means ±SEM. The significance of differences for GTPCH activity and biopterin levels was determined using the paired t-test with statistical significance at a p value of <0.05. The statistical analysis of NO₂− levels was determined using ANOVA. Statistical significance was established at a p value <0.01.

### 8.2. Results

3T3 cells were used along with RSMC to test the efficacy of GTPCH gene transfer. Lipofectamine transfection of 3T3, 3T3-iNOS, and RSMC resulted in a gene transfer efficiency of approximately 1 % as determined by X-gal staining for β-galactosidase activity in pIEP-lacZ transfected cells. Northern blot analysis, using a human GTPCH cDNA probe that crosshybridizes with rodent GTPCH, revealed no endogenous GTPCH expression in either 3T3-iNOS or RSMC groups (FIG. 22). Endogenous GTPCH transcripts measure over 3 kb in size as seen in human hepatocytes (FIG. 22, lane 1) which are abundant sources of GTPCH. However, recombinant GTPCH mRNA measures approximately 900 bp in size and was only detected in pCIS-GTPCH transfected cells. A larger 1.2 kb mRNA signal was detected in an groups which does not represent a GTPCH signal because it was not detectable using a rat GTPCH cDNA probe. The identity of this signal is not known. These data show that the transferred GTPCH gene is successfully transcribed. To confirm that functional GTPCH enzyme can be generated, measurements of GTPCH enzymatic activity were performed and are summarized in Table 3. Control transfected 3T3 cells uniformly lacked GTPCH activity while pCIS-GTPCH transfected cells demonstrated levels of activity varying between 30-170 pmol/h/mg protein which are of comparable magnitude to that measured in hepatocytes, cells which constitutively express GTPCH. The variability in GTPCH activity was most likely secondary to slight variations in transfection efficiencies from experiment to experiment. The intracellular biopterins (BH₄+BH₂+biopterins) generated by GTPCH gene transfer into 3T3 types cells and RAOSMCs are also summarized in Table 3. Dramatic increases in total intracellular biopterin concentrations were measured in pCIS-GTPCH transfected cells, regardless of the cell type. The small amount of biopterins measured in control cells was most likely due to the uptake of biopterins present in the serum in the growth media. These data indicate that low efficiency GTPCH gene transfer results in high level expression of functional GTPCH and completes the de novo BH₄ biosynthetic pathway in RSMC and 3T3 cells with the consequent generation of significant intracellular biopterins.

The ability of GTPCH gene transfer to reconstitute iNOS activity was assessed in 3T3iNOS cells. 3T3-iNOS cells were transfected with either pIEP-lacZ or pCIS-GTPCH and subsequent NO synthesis was measured by NO₂ accumulation in the culture supernatant. The efficiency of GTPCH expression at supporting iNOS activity in these cells was compared to the maximal NO synthesis achieved by exogenous BH₄ supplementation (FIG. 23). Transfection of 3T3-iNOS with pIEP-lacZ resulted in little NO₂ accumulation (3.9±0.4 nmol/mg protein/24 h) and did not attenuate the response to exogenous BH₄ (223.6 +18.9). In contrast, cells transfected with pCIS-GTpCH generated NO₂ levels comparable to that achieved with exogenous BH₄ (176.1±3.8 v.s 210.2±10.0, respectively). This result was surprising given the low transfection efficiency.

MTX was added to the growth medium to show the mechanism by which low efficiency GTPCH gene transfer could sustain iNOS activity in a whole population of cells. MTX inhibits dihydrofolate reductase (DHFR) which can convert dihydrobiopterin (BH,), a breakdown product of BH₄, back to the active form of the cofactor. MTX reduced the amount of iNOS activity recovered by BH₄ supplementation by over 5 fold (FIG. 23), indicating the majority of exogenous BH₄ enters cells in a form that requires metabolism by DHFR. In pCIS-GTPCH transfected 3T3iNOS cells, the MTX effect was less pronounced and only reduced iNOS activity by 50%, suggesting that BH₄ synthesized within cells can reach other cells as BH₄. Culturing 3T3-iNOS cells with conditioned medium collected from GTPCH expressing 3T3 cells, which should contain released biopterins, only reconstituted 25% of maximal iNOS activity. The requirement for direct cell:cell contact for BH₄ transfer was then examined by co-culturing 3T3iNOS cells with 3T3 transfected with either pIEP-lacZ or pCIS-GTPCH plasmids. Co-culturing of 3T3-iNOS cells with pIEP-lacZ transfected 3T3 cells resulted in minimal NO₂− accumulation (FIG. 24) and indicated the co-culturing process did not stimulate endogenous GTPCH activity. However, maximal iNOS activity was recovered when 3T3-iNOS cells were cocultured with pCIS-GTPCH transfected 3T3 cells, and this activity could not be further augmented by exogenous BH₄ (FIG. 24). These data show that iNOS and GTPCH do not have to coexist in the same cell for the benefit of BH₄ biosynthesis to be realized. Only a few cells expressing GTPCH and synthesizing the cofactor can optimally support iNOS activity in a large population of cells.

### 8.3. Discussion

This example shows the feasibility of GTPCH gene transfer as a method of delivering BH₄ to support iNOS activity in cofactor-deficient cells. GTPCH gene transfer was accomplished in both murine fibroblast NIH3T3 cells and RSMC with low efficiency. The exemplified low efficiency of gene transfer increases both GTPCH activity and intracellular biopterin levels dramatically. In addition, this level of gene transfer is sufficient to reconstitute maximal iNOS activity in a population of 3T3 cells all expressing the iNOS enzyme. Thus, BH₄ synthesized in one cell may be accessible to neighboring cells and can adequately support iNOS function in those cells. This phenomenon may be facilitated by direct transport of BH₄ between communicating cells, and to a lesser extent, through extracellular diffusion and uptake of biopterins by distant cells. These results suggest that a small number of cells possessing GTPCH activity can synthesize adequate quantities of the cofactor to support iNOS activity in a larger number of cells. The ultimate success of GTPCH gene transfer to deliver BH₄ as an adjuvant to iNOS gene therapy may rest in the fact that GTPCH and iNOS activities do not have to reside in the same cells for the benefit of GTPCH activity to be manifested.

In vivo BH₄ delivery may be accomplished in one of two ways. One is through the direct administration of BH₄ or the other substrate for BH₄ biosynthesis, sepiapterin. These compounds have been efficacious in repleting the cofactor in patients with phenylketonuria arising from a primary defect in GTPCH, a relatively rare cause of this disease (Kaufman, et al., 1978, New Engl. J. Med. 299: 673-679). Alternatively, GTPCH gene transfer is a viable option. For iNOS gene therapy considerations, to simultaneously deliver a second gene does not involve additional manipulations or risk. Simultaneous GTPCH delivery may lead to BH₄ synthesis only in the location where it is needed and for the same duration as iNOS expression. It can be imagined that only a few SMCs in the thick medial layer of the arterial wall may be successfully transduced in vivo. However, these results show that BH₄ synthesized in one cell can be transported to neighboring cells where the transferred iNOS may exist.

### 9. Example

### Biologic Therapy for Treating Non-healing Wounds

This example shows that addition of AdiNOS promotes angiogenesis in both wild type mice and mice deleted for the iNOS gene. Mice were were anesthetized with nembutal and a 2×2 cm full thickness external wound was generated on B6×129 mice with an iNOS knockout. A supernatant containing 10⁷ PFU AdiNOS was dripped onto the wound and was washed away from the wound site 30 minutes after application. Control mice were not subjected to AdiNOS administration. The animals were revived and wounds were inspected every 2 days for evidence of complete wound closure. The average time for complete closure for a control wild type mouse was 17 days as compared to 15.5 days for a wild type mouse plus AdiNOS. Additionally, the iNOS knockout mice took a full 23 days to show complete wound closure compared to 19 days for the iNOS knockout plus AdiNOS. Therefore, this data shows a propensity for faster healing when administered an iNOS source compared to a control wherein a source of iNOS is not supplied.

### 10. Deposit of Microorganisms

The following microorganisms have been deposited under the Budapest Treaty by David A. Geller on behalf of the University of Pittsburgh of the Commonwealth System of Higher Education, Pittsburgh, Pa. 15260, USA, on Nov. 18, 1992, with and are available from the permanent collection of the American Type Culture Collection (ATCC), 12301 Parklawn Drive, Rockville, Md. 202852-1776, USA:

ATCC 75358—Human Hepatocyte Inducible Nitric Oxide Synthase cDNA in pBluescript (pHiNOS)

ATCC 69126—Human Hepatocyte Inducible Nitric Oxide Synthase cDNA in pBluescript transformed in E. coli SOLR bacteria (plasmid HiNOS cDNA)

The American Type Culture Collection has performed viability tests on each of the hereinbefore mentioned deposited microorganisms and has concluded on Nov. 20, 1992, that each of the hereinbefore mentioned deposited microorganisms is viable and capable of reproduction.

TABLE 1
Rat smooth muscle cell nitric oxide synthesis and proliferation
following AdiNOS infection.

| | Nitrite | | |
|---|---|---|---|
| | (nmol/mg/ | [³H]-thymidine | Proliferation |
| Treatment | 24h) | incorporation (CMP) | % of uninfected |
| AdlacZ | 5 ± 2 | 16,102 ± 668 | 100 |
| AdlacZ + BH₄ | 2 ± 1 | 15,919 ± 491 | 99 |
| AdiNOS | 116 ± | 6,623 ± 285** | 41 |
| | 10** | | |
| AdiNOS + BH₄ | 471 ± 55* | 4,070 ± 157* | 25 |
| AdiNOS + BH_{4 +} | 77 ± 15 | 6,386 ± 277 | 40 |
| NMA | | | |

Values = mean ± SD, n = 3
NMA = N^{G}-monomethyl-L-arginine (1 mM)
BH₄ = tetrahydrobiopterin (10 μM)
*p < 0.001 vs. all other groups by ANOVA
**p < 0.001 vs. controls by ANOVA

TABLE 2
Total Nitrogen Oxide and
cGMP Production By Porcine Arterial Segments.

| | | | cGMP | |
|---|---|---|---|---|
| Treatment | Total NO₂ and NO₃ | p value^{∓} | (fmol/mg/ | p value^{∓} |
| Groups | (pmoUmg/24 h) | (ANOVA) | 24 h) | (ANOVA) |
| Control | 253.7 ± 6.5* | — | 5.2 ± 2.8 | — |
| Injury alone | 35.4 ± 8.4 | NS | 7.3 ± 3.4 | NS |
| Injury + | 40.1 ± 5.2 | NS | 6.8 ± 2.9 | NS |
| MFG1acZ | | | | |
| Injury + | 121.9 ± 43.1 | 0.001 | 101.3 ± 12.1 | 0.002 |
| DFGiNOS | | | | |
| Injury + | 37.4 ± 8.2 | NS | 5.6 ± 4.2 | NS |
| DFGiNOS + | | | | |
| L-NMA* | | | | |

*Values are means ± standard deviations, n = 4, representative of 3 separate experiments.
^{∓}Versus uninjured control arterial segments

TABLE 3
GTPCH and Total Biopterin. Transfected NIH3T3 and RSMC

| Cell Type + Transfected | GTPCH Activity^{≃} | p value^{τ} | Total Biopteins* | pvalue** |
|---|---|---|---|---|
| DNA | (pmol/h/mg) | paired t-test | (pmol/mg) | paired t-test |
| 3T3 + pIEP-lacZ | 0 ± 0 | — | 3.0 ± 0.6 | — |
| 3T3 + pCIS-GTPCH | 169.3 ± 6.6 | 0.001 | 60.6 ± 2.6 | 0.001 |
| 3T3-iNOS + pIEP-lacZ | 0 ± 0 | — | 1.3 ± 0.6 | — |
| 3T3-iNOS + pCIS-GTPCH | 36.1 ± 6.4 | 0.01 | 25.7 ± 5.6 | 0.05 |
| RSMC + pIEP-lacZ | N.D.^{§} | — | 1.8 ± 1.3 | — |
| RSMC + pCIS-GTPCH | N.D. | — | 101.7 ± 28.3 | 0.001 |

*Values are means ± standard error. n = 3, representative of 3 separate experiments
^{τ}Versus pIEP-lacZ transfected control cells
**Versus pIEP-lacZ transfected control cells
^{§}Not done

### Incorporation By Reference

All references, including publications, patent applications, and patents, cited herein are hereby incorporated by reference to the same extent as if each reference were individually and specifically indicated to be incorporated by reference and were set forth in its entirety herein. In addition, the following United States patents are also incorporated herein by specific reference thereto: U.S. Pat. Nos. 6,103,230, 5,882,908, 5,830,461, 5,714,511, 5,658,565, and 5,468,630.

### Interpretive Guidelines

The use of the terms “a” and “an” and “the” and similar referents in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Of course, variations of those preferred embodiments will become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A pharmaceutical composition comprising as an active ingredient a pharmaceutical agent comprising a DNA sequence that codes for a protein which possesses the biological activity of inducible nitrogen monoxide synthase (iNOS) and eukaryotic regulation elements, wherein said eukaryotic regulation elements result in the expression of said DNA sequence in eukaryotic cells, and a pharmaceutically acceptable carrier.

2. The pharmaceutical composition according to claim 1, wherein the DNA sequence that codes for a protein which possesses the biological activity of inducible nitrogen monoxide synthase (iNOS) is a cDNA sequence.

3. The pharmaceutical composition according to claim 1 or 2, wherein the DNA or cDNA sequence is derived from mammals.

4. The pharmaceutical composition according to claim 3, wherein the DNA or cDNA sequence constitutes a human DNA or cDNA sequence.

5. The pharmaceutical composition according to claim 1, wherein said eukaryotic regulation elements are derived from the cytomegalovirus (CMV) promoter and/or enhancer of the early gene.

6. The pharmaceutical composition according to claim 1, wherein said eukaryotic regulation elements are derived from an eukaryotic virus.

7. The pharmaceutical composition according to claim 1, wherein said DNA expression vector represents pSCMV-iNOS.

8. The pharmaceutical composition according to claim 1, wherein said eukaryotic regulation elements are derived from an adenovirus promoter and/or enhancer element.

9. A pharmaceutical composition comprising as an active ingredient a pharmaceutical agent comprising the plasmid pSCMV-iNOS which contains a DNA sequence that codes for a protein which possesses the biological activity of inducible nitrogen monoxide synthase (iNOS) and eukaryotic regulation elements, wherein said eukaryotic regulations elements result in the expression of said DNA sequence in eukaryotic cells, and a pharmaceutically acceptable carrier.

10. A pharmaceutical composition for the treatment and prevention of high blood pressure, arteriosclerosis, stenosis and restenosis comprising as an active ingredient a DNA expression vector which comprises a DNA sequence encoding inducible nitrogen monoxide synthase (iNOS) and eukaryotic regulation elements, wherein the expression vector is complexed to liposomes.

11. The pharmaceutical composition as claimed in claim 10, wherein the DNA sequence encoding inducible nitrogen monoxide synthase (iNOS) is a cDNA sequence.

12. The pharmaceutical composition as claimed in claim 10, wherein the expression vector further comprises a DNA sequence that allows replication in bacteria, a DNA sequence encoding the SV40 replication sequence element and a polyadenylation signal.

13. The pharmaceutical composition as claimed in claim 10, wherein the eukaryotic regulation elements are derived from a eukaryotic virus.

14. The pharmaceutical composition as claimed in claim 10, wherein the DNA expression vector is pSCMV-iNOS.

15. The pharmaceuticals composition as claimed in claims 10 or 11, wherein the DNA or cDNA sequence is a mammalian sequence.

16. The pharmaceutical composition as claimed in claim 13, wherein the eukaryotic virus is Cytomegalovirus or Adenovirus.

17. The pharmaceutical composition as claimed in claim 15, wherein the DNA or cDNA sequence constitutes a human DNA or cDNA sequence.

18. The pharmaceutical composition as claimed in claim 15, wherein the DNA or cDNA sequence constitutes a mouse DNA or cDNA sequence.

19. A pharmaceutical composition for the treatment and prevention of vascular disorders which comprises a pharmaceutically acceptable carrier and, as an active ingredient, a pharmaceutical agent comprising a DNA sequence encoding inducible nitrogen monoxide synthase (iNOS) and eukaryotic regulation elements which result in the expression of said DNA sequence in eukaryotic cells, wherein the pharmaceutical agent is complexed to liposomes.

20. The pharmaceutical composition as claimed in claim 19, wherein the DNA sequence encoding inducible nitrogen monoxide synthase (iNOS) is a cDNA sequence.

21. The pharmaceutical composition as claimed in claim 19, wherein the eukaryotic regulation elements are derived from a eukaryotic virus.

22. The pharmaceutical composition according to claim 19, wherein the eukaryotic regulation elements are derived from an Adenovirus promoter and/or enhancer element.

23. The pharmaceutical composition as claimed in claim 19, wherein the eukaryotic regulation elements are derived from a Cytomegalovirus (CMV) promoter and/or enhancer of the immediate early gene.

24. The pharmaceutical composition as claimed in claim 19 or 20, wherein the DNA or cDNA sequence is a mammalian sequence.

25. The pharmaceutical composition as claimed in claim 21, wherein the eukaryotic virus is Cytomegalovirus or Adenovirus.

26. The pharmaceutical composition as claimed in claim 24, wherein the DNA or cDNA sequence constitutes a human DNA or cDNA sequence.

27. The pharmaceutical composition as claimed in claim 24, wherein the DNA or cDNA sequence constitutes a mouse DNA or cDNA sequence.

28. A pharmaceutical composition comprising as an active ingredient a pharmaceutical agent comprising a plasmid which contains a DNA sequence that codes for a protein which possesses the biological activity of inducible nitrogen monoxide synthase (iNOS) and eukaryotic regulation elements comprising the human CMV immediate early promoter/enhancer, wherein said eukaryotic regulations elements result in the expression of said DNA sequence in eukaryotic cells, and a pharmaceutically acceptable carrier.


SEQUENCE LISTING (2 sequences)

SEQ ID NO: 1 - DNA, 4145 bp, Induced Human Hepatocyte RNA
  CDS (207)..(3668)
ctgctttaaaatctctcggccacctttgatgaggggactgggcagttctagacagtcccgaagttctcaaggcacaggtctcttcctggtttgactgtccttaccccggggaggcagtgcagccagctgcaagccccacagtgaagaacatctgagctcaaatccagataagtgacataagtgacctgctttgtaaagccatagagatggcctgtccttggaaatttctgttcaagaccaaattccaccagtatgcaatgaatggggaaaaagacatcaacaacaatgtggagaaagccccctgtgccacctccagtccagtgacacaggatgaccttcagtatcacaacctcagcaagcagcagaatgagtccccgcagcccctcgtggagacgggaaagaagtctccagaatctctggtcaagctggatgcaaccccattgtcctccccacggcatgtgaggatcaaaaactggggcagcgggatgactttccaagacacacttcaccataaggccaaagggattttaacttgcaggtccaaatcttgcctggggtccattatgactcccaaaagtttgaccagaggacccagggacaagcctacccctccagatgagcttctacctcaagctatcgaatttgtcaaccaatattacggctccttcaaagaggcaaaaatagaggaacatctggccagggtggaagcggtaacaaaggagatagaaacaacaggaacctaccaactgacgggagatgagctcatcttcgccaccaagcaggcctggcgcaatgccccacgctgcattgggaggatccagtggtccaacctgcaggtcttcgatgcccgcagctgttccactgcccgggaaatgtttgaacacatctgcagacacgtgcgttactccaccaacaatggcaacatcaggtcggccatcaccgtgttcccccagcggagtgatggcaagcacgacttccgggtgtggaatgctcagctcatccgctatgctggctaccagatgccagatggcagcatcagaggggaccctgccaacgtggaattcactcagctgtgcatcgacctgggctggaagcccaagtacggccgcttcgatgtggtccccctggtcctgcaggccaatggccgtgaccctgagctcttcgaaatcccacctgaccttgtgcttgaggtggccatggaacatcccaaatacgagtggtttcgggaactggagctaaagtggtacgccctgcctgcagtggccaacatgctgcttgaggtgggcggcctggagttcccagggtgccccttcaatggctggtacatgggcacagagatcggagtccgggacttctgtgacgtccagcgctacaacatcctggaggaagtgggcaggagaatgggcctggaaacgcacaagctggcctcgctctggaaagaccaggctgtcgttgagatcaacattgctgtgatccatagttttcagaagcagaatgtgaccatcatggaccaccactcggctgcagaatccttcatgaagtacatgcagaatgaataccggtcccgtgggggctgcccggcagactggatttggctggtccctcccatgtctgggagcatcacccccgtgtttcaccaggagatgctgaactacgtcctgtcccctttctactactatcaggtagaggcctggaaaacccatgtctggcaggacgagaagcggagacccaagagaagagagattccattgaaagtcttggtcaaagctgtgctctttgcctgtatgctgatgcgcaagacaatggcgtcccgagtcagagtcaccatcctctttgcgacagagacaggaaaatcagaggcgctggcctgggacctgggggccttattcagctgtgccttcaaccccaaggttgtctgcatggataagtacaggctgagctgcctggaggaggaacggctgctgttggtggtgaccagtacgtttggcaatggagactgccctggcaatggagagaaactgaagaaatcgctcttcatgctgaaagagctcaacaacaaattcaggtacgctgtgtttggcctcggctccagcatgtaccctcggttctgcgcctttgctcatgacattgatcagaagctgtcccacctgggggcctctcagctcaccccgatgggagaaggggatgagctcagtgggcaggaggacgccttccgcagctgggccgtgcaaaccttcaaggcagcctgtgagacgtttgatgtccgaggcaaacagcacattcagatccccaagctctacacctccaatgtgacctgggacccgcaccactacaggctcgtgcaggactcacagcctttggacctcagcaaagccctcagcagcatgcatgccaagaacgtgttcaccatgaggctcaaatctcggcagaatctacaaagtccgacatccagccgtgccaccatcctggtggaactctcctgtgaggatggccaaggcctgaactacctgccgggggagcaccttggggtttgcccaggcaaccagccggccctggtccaaggcatcctggagcgagtggtggatggccccacaccccaccagacagtgcgcctggaggacctggatgagagtggcagctactgggtcagtgacaagaggctgcccccctgctcactcagccaggccctcacctactccccggacatcaccacacccccaacccagctgctgctccaaaagctggcccaggtggccacagaagagcctgagagacagaggctggaggccctgtgccagccctcagagtacagcaagtggaagttcaccaacagccccacattcctggaggtgctagaggagttcccgtccctgcgggtgtctgctggcttcctgctttcccagctccccattctgaagcccaggttctactccatcagctcctcccgggatcacacgcccacggagatccacctgactgtggccgtggtcacctaccacaccggagatggccagggtcccctgcaccacggtgtctgcagcacatggctcaacagcctgaagccccaagacccagtgccctgctttgtgcggaatgccagcgccttccacctccccgaggatccctcccatccttgcatcctcatcgggcctggcacaggcatcgtgcccttccgcagtttctggcagcaacggctccatgactcccagcacaagggagtgcggggaggccgcatgaccttggtgtttgggtgccgccgcccagatgaggaccacatctaccaggaggagatgctggagatggcccagaagggggtgctgcatgcggtgcacacagcctattcccgcctgcctggcaagcccaaggtctatgttcaggacatcctgcggcagcagctggccagcgaggtgctccgtgtgctccacaaggagccaggccacctctatgtttgcggggatgtgcgcatggcccgggacgtggcccacaccctgaagcagctggtggctgccaagctgaaattgaatgaggagcaggtcgaggactatttctttcagctcaagagccagaagcgctatcacgaagatatcttcggtgctgtatttccttacgaggcgaagaaggacagggtggcggtgcagcccagcagcctggagatgtcagcgctctgagggcctacaggaggggttaaagctgccggcacagaacttaaggatggagccagctctgcattatctgaggtcacagggcctggggagatggaggaaagtgatatcccccagcctcaagtcttatttcctcaacgttgctccccatcaagccctttacttgacctcctaacaagtagcaccctggattgatcggagcctcctctctcaaactggggcctccctggtcccttggagacaaaatcttaaatgccaggcctggcgagtgggtgaaagatggaacttgctgctgagtgcaccacttcaagtgaccaccaggaggtgctatcgcaccactgtgtatttaactgccttgtgtacagttatttatgcctctgtatttaaaaaactaacacccagtctgttccccatggccacttgggtcttccctgtatgattccttgatggagatatttacatgaattgcattttactttaatc

SEQ ID NO: 2 - protein, 1153 aa, Induced Human Hepatocyte RNA [sequence omitted]